(19) United States
(12) Patent Application Publication
(10) Pub. No.: US 2025/0186466 A1
(43) Pub. Date: Jun. 12, 2025

(54) ZLT-007 AND METHODS OF TREATING DIABETIC NEUROPATHY

(71) Applicant: IIere Therapeutics LLC, Newtown Square, PA (US)

(72) Inventors: Oludare ODUMOSU, Newtown Square, PA (US); Christopher Hill, Newtonwn Square, PA (US)

(21) Appl. No.: 19/016,566

(22) Filed: Jan. 10, 2025

Related U.S. Application Data
(63) Continuation of application No. PCT/US2023/069951, filed on Jul. 11, 2023.
(60) Provisional application No. 63/388,178, filed on Jul. 11, 2022.

(51) Int. Cl. A61K 31/00; A61K 9/20; A61K 9/48; A61K 31/01; A61K 31/045; A61P 25/02

(52) U.S. Cl. CPC A61K 31/658; A61K 9/2009; A61K 9/2013; A61K 9/4825; A61K 9/485; A61K 9/4858; A61K 31/01; A61K 31/045; A61P 25/02

(57) ABSTRACT

The present disclosure provides compositions containing Cannabinoid Products and methods of making the same. The disclosure also provides tablets and capsules containing Cannabinoid Products and methods of making the tablets and capsules. The disclosure also provides pharmaceutical products for treating diabetic ailments including diabetic neuropathy.

### CROSS-REFERENCE TO RELATED APPLICATIONS

This application is a continuation of International Application No. PCT/US2023/069951, filed Jul. 11, 2023, which claims benefit of priority to U.S. Provisional Patent Application No. 63/388,178, filed Jul. 11, 2022, the entire contents of which are incorporated herein by reference in there entireties for all purposes.

### FIELD

The present disclosure provides Cannabinoid Products and tablets and capsules containing Cannabinoid Products. The disclosure also provides methods of making said Cannabinoid Products and tablets and capsules, and methods of using said products in the treatment of various diseases/ailments.

### BACKGROUND

Roughly 37 million Americans suffer from diabetes, with rates rising every year. A large number of drugs have been developed to treat the disease, including those that promote release of insulin from the pancreas, uptake of glucose from the blood, and reduction in the level of glucose production. Unfortunately, these treatments generally only slow the progression of type II diabetes, and do not yet provide a cure. Diabetes can further manifest itself as neuropathy, peripheral neuropathy, retinopathy and cardiovascular disease. Thus, there is a need in the art for additional pharmaceuticals to treat diabetic symptoms, until a cure is found.

### SUMMARY

The present disclosure provides Cannabinoid Products containing cannabinoids and methods of making Cannabinoid Products. The disclosure also provides tablets and capsules comprising Cannabinoid Products and methods of making the tablets and capsules. When a Cannabinoid Product described herein is compressed to form a tablet or encapsulated in a shell to form a capsule, the cannabinoids do not separate from the composition. In some embodiments, the present disclosure provides ZLT-7 pharmaceutical for treating diabetic neuropathy.

Provided herein is a Cannabinoid Product comprising: (a) a cannabinoid oil; and (b) colloidal silicon dioxide particles. Provided herein is a Cannabinoid Product comprising: (a) a cannabinoid oil; (b) colloidal silicon dioxide particles; (c) a filler comprising silicified microcrystalline cellulose or mannitol; (d) a disintegrant comprising sodium starch glycolate or croscarmellose sodium; and (e) a lubricant comprising sodium stearyl fumarate. Provided herein is a Cannabinoid Product comprising: (a) a cannabinoid oil; (b) colloidal silicon dioxide particles; and (c) a lubricant comprising sodium stearyl fumarate.

In embodiments, the Cannabinoid Product comprises from about 10% to about 60% cannabinoid oil by weight. In embodiments, the Cannabinoid Product comprises from about 0.5% to about 20% cannabinoid oil by weight. In embodiments, the Cannabinoid Product comprises from about 1% to about 10% cannabinoid oil by weight. In embodiments, the Cannabinoid Product comprises about 2% cannabinoid oil by weight. In embodiments, the Cannabinoid Product comprises about 8% cannabinoid oil by weight. In embodiments, the Cannabinoid Product comprises from about 19.4% to about 41.9% cannabinoid oil by weight. In embodiments, the filler is silicified microcrystalline cellulose. In embodiments, the Cannabinoid Product comprises from about 30% to about 60% silicified microcrystalline cellulose by weight.

In embodiments, the Cannabinoid Product comprises from about 20% to about 45% silicified microcrystalline cellulose by weight. In embodiments, the Cannabinoid Product comprises about 46.6% silicified microcrystalline cellulose by weight. In embodiments, the Cannabinoid Product comprises about 25% silicified microcrystalline cellulose by weight. In embodiments, the filler is mannitol. In embodiments, the Cannabinoid Product comprises from about 5% to about 60% mannitol by weight. In embodiments, the Cannabinoid Product comprises from about 12.3% to about 43.4% mannitol by weight. In embodiments, the Cannabinoid Product comprises from about 20% to about 70% colloidal silicon dioxide particles by weight. In embodiments, the Cannabinoid Product comprises from about 29.1% to about 58.2% colloidal silicon dioxide particles by weight. In embodiments, the Cannabinoid Product comprises from about 25% to about 50%, from about 25% to about 35%, or from about 35% to about 45% colloidal silicon dioxide particles by weight. In embodiments, the Cannabinoid Product comprises about 32% colloidal silicon dioxide particles by weight.

In embodiments, the Cannabinoid Product comprises about 39% colloidal silicon dioxide particles by weight. In embodiments, the Cannabinoid Product comprises a disintegrant. In embodiments, the disintegrant is sodium starch glycolate. In embodiments, the Cannabinoid Product comprises from about 0.5% to about 10% sodium starch glycolate by weight. In embodiments, the Cannabinoid Product comprises from about 2.0% to about 5.5% sodium starch glycolate by weight. In embodiments, the disintegrant is croscarmellose sodium. In embodiments, the Cannabinoid Product comprises from about 0.5% to about 5% croscarmellose sodium by weight. In embodiments, the Cannabinoid Product comprises about 1.9% croscarmellose sodium by weight. In embodiments, the Cannabinoid Product comprises from about 0.5% to about 10% sodium stearyl fumarate by weight. In embodiments, the Cannabinoid Product comprises from about 1% to about 2.7% sodium stearyl fumarate by weight. In embodiments, the Cannabinoid Product comprises from about 0.1% to about 1% sodium stearyl fumarate by weight.

In embodiments, the Cannabinoid Product comprises about 0.4% sodium stearyl fumarate by weight. In embodiments, the Cannabinoid Product comprises. In embodiments, the Cannabinoid Product comprises: (a) about 41.9% cannabinoid oil by weight; (b) about 41.9% colloidal silicon dioxide particles by weight; (c) about 12.3% mannitol by weight; (d) about 2% sodium starch glycolate by weight; and (e) about 2% sodium stearyl fumarate by weight. In embodiments, the Cannabinoid Product comprises: (a) about 22.1% cannabinoid oil by weight; (b) about 58.2% colloidal silicon dioxide particles by weight; (c) about 14.3% mannitol by weight; (d) about 2.7% sodium starch glycolate by weight; and (e) about 2.7% sodium stearyl fumarate by weight. In embodiments, the Cannabinoid Product comprises: (a) about 22.1% cannabinoid oil by weight; (b) about 29.1% colloidal silicon dioxide particles by weight; (c) about 40.7% mannitol by weight; (d) about 5.5% sodium starch glycolate by weight; and (e) about 2.7% sodium stearyl fumarate by weight. In embodiments, the Cannabinoid Product comprises.

In embodiments, the Cannabinoid Product comprises (a) about 22.1% cannabinoid oil by weight; (b) about 29.1% colloidal silicon dioxide particles by weight; (c) about 43.4% mannitol by weight; (d) about 2.7% sodium starch glycolate by weight; and (e) about 2.7% sodium stearyl fumarate by weight. In embodiments, the Cannabinoid Product comprises: (a) about 19.4% cannabinoid oil by weight; (b) about 31.1% colloidal silicon dioxide particles by weight; (c) about 46.6% silicified microcrystalline cellulose by weight; (d) about 1.9% croscarmellose sodium by weight; and (e) about 1% sodium stearyl fumarate by weight. In embodiments, the Cannabinoid Product comprises: (a) about 2.2% cannabinoid oil by weight; (b) about 32.4% colloidal silicon dioxide particles by weight; (c) about 0.4% sodium stearyl fumarate by weight; and (d) about 34.8% silicified microcrystalline cellulose by weight. In embodiments, the Cannabinoid Product comprises: (a) about 8.1% cannabinoid oil by weight; (b) about 39% colloidal silicon dioxide particles by weight; (c) about 0.4% sodium stearyl fumarate by weight; (d) about 40.4% cannabidiol (CBD) isolate by weight; and (e) about 11.7% cannabigerol (CBG) isolate by weight.

In embodiments, the Cannabinoid Product comprises: (a) about 8.1% cannabinoid oil by weight; (b) about 39% colloidal silicon dioxide particles by weight; (c) about 0.4% sodium stearyl fumarate by weight; (d) about 40.4% cannabidiol (CBD) isolate by weight; (e) about 11.7% cannabigerol (CBG) isolate by weight; (f) about 0.2% myrcene by weight; and (g) about 0.2% linalool by weight. In embodiments, the Cannabinoid Product comprises: (a) about 2.2% cannabinoid oil by weight of the Cannabinoid Product; (b) about 32.4% colloidal silicon dioxide particles by weight; and (c) about 0.4% sodium stearyl fumarate by weight. In embodiments, the Cannabinoid Product comprises: (a) about 8.1% cannabinoid oil by weight; (b) about 39% colloidal silicon dioxide particles by weight; and (c) about 0.4% sodium stearyl fumarate by weight. In embodiments, the Cannabinoid Product comprises a cannabinoid isolate.

In embodiments, the cannabinoid isolate is cannabidiol (CBD) isolate or cannabigerol (CBG) isolate. In embodiments, the Cannabinoid Product comprises from about 20% to about 60%, from about 20% to about 40%, or from about 40% to about 60% cannabinoid isolate by weight. In embodiments, the Cannabinoid Product comprises a flavoring. In embodiments, the flavoring is a terpene. In embodiments, the terpene is myrcene, linalool, or a combination thereof. In embodiments, the cannabinoid oil is a cannabinoid distillate.

In embodiments, provided herein is a Cannabinoid Product comprising a cannabinoid oil, colloidal silicon dioxide particles, and optionally filler, disintegrant, or lubricant, wherein the cannabinoid oil and the colloidal silicon dioxide particles are mixed in a high-shear mixer before the addition of the filler, disintegrant, or lubricant. In embodiments, provided herein is a Cannabinoid Product comprising a cannabinoid oil, colloidal silicon dioxide particles, and a lubricant; wherein the cannabinoid oil and the colloidal silicon dioxide particles are mixed in a high-shear mixer before the addition of the lubricant. In embodiments, provided herein is a Cannabinoid Product; wherein the lubricant is added after the filler and disintegrant. In embodiments, provided herein is a Cannabinoid Product; wherein the lubricant is added after the cannabinoid oil and colloidal silicon dioxide particles. In embodiments, the colloidal silicon dioxide particles of a Cannabinoid Product described herein have an average particle size of 20-60 μm. In embodiments, the colloidal silicon dioxide particles of a Cannabinoid Product described herein contain pores with a volume of 1.5-1.9 milliliter (mL)/gram (g).

In embodiments, provided herein is a tablet comprising any Cannabinoid Product described herein. In embodiments, the tablet has a hardness of from 50 Newtons (N) to 100 N. In embodiments, the tablet has a hardness of about 65 N. In embodiments, the tablet has a compression force of from 1 kiloNewtons (kN) to 30 kN. In embodiments, the tablet has a compression force of from about 10 kN to 16 kN. In embodiments, at least 18% of the tablet dissolves in fed state simulated intestinal fluid or fasted state simulated intestinal fluid within one hour. In embodiments, from 60% to about 80% of the tablet dissolves in fed state simulated intestinal fluid or fasted state simulated intestinal fluid within one hour. In embodiments, from 70% to about 80% of the tablet dissolves in fed state simulated intestinal fluid or fasted state simulated intestinal fluid within one hour. In embodiments, at least 36.5% of the tablet dissolves in polysorbate 80 in one hour. In embodiments, the tablet disintegrates in from 1 minute to about 10 minutes in 2% w/v sodium lauryl sulfate. In embodiments, the tablet dissolves at least two times, at least three times, at least four times, at least five times, or at least ten times faster than a tablet containing a Cannabinoid Product lacking colloidal silicon dioxide particles. In embodiments, the tablet has a weight of from about 100 to about 800 mg. In embodiments, the tablet comprises from about 0.1% to about 30% of cannabinoid oil by weight. In embodiments, the tablet is an orodispersable tablet.

In embodiments, provided herein is a capsule comprising any Cannabinoid Product described herein. In embodiments, the capsule comprises a shell comprising gelatin or hydroxypropylmethylcellulose. In embodiments, the capsule disintegrates within about 2 minutes in an aqueous solvent. In embodiments, at least 18% of the capsule dissolves in fed state simulated intestinal fluid or fasted state simulated intestinal fluid within one hour. In embodiments, from 18% to about 60% of the capsule dissolves within one hour in fed state simulated intestinal fluid or fasted state simulated intestinal fluid. In embodiments, from 18% to about 42.3% of the capsule dissolves within one hour in fed state simulated intestinal fluid or fasted state simulated intestinal fluid. In embodiments, at least 36.5% of the capsule dissolves in fed state simulated intestinal fluid or fasted state simulated intestinal fluid within one hour. In embodiments, the capsule dissolves at least two times, at least three times, at least four times, at least five times, or at least ten times faster than a capsule containing a Cannabinoid Product lacking colloidal silicon dioxide particles. In embodiments, the capsule comprises from about 1% to about 50% cannabinoid oil by weight.

In embodiments, provided herein is a method of making a Cannabinoid Product described herein, comprising: (a) mixing the cannabinoid oil and colloidal silicon dioxide particles; (b) adding the filler and disintegrant to the mixture of distillate and colloidal silicon dioxide particles; and (c) adding the lubricant. In embodiments, step (a) is performed before step (b), and step (b) is performed before step (c). In embodiments, the cannabinoid oil is heated before the extract oil is mixed with the colloidal silicon dioxide particles. In embodiments, the cannabinoid oil and colloidal silicon dioxide particles are heated after mixing. In embodiments, the cannabinoid oil is diluted in a solvent before mixing with colloidal silicon dioxide particles. In embodiments, the solvent is ethanol. In embodiments, the cannabinoid oil is heated to at least 30° C., at least 40° C., at least 50° C., at least 60° C., at least 70° C., at least 80° C., or at least 90° C. In embodiments, the cannabinoid oil is added to the colloidal silicon dioxide particles. In embodiments, the colloidal silicon dioxide particles are added to the cannabinoid distillate. In embodiments, the cannabinoid oil and colloidal silicon dioxide particles are mixed in a high-shear mixer. In embodiments, the cannabinoid oil and colloidal silicon dioxide particles are mixed for at least 30 seconds, at least 1 minute, at least 5 minutes, at least 10 minutes, at least 15 minutes, or at least 20 minutes. In embodiments, the cannabinoid oil and colloidal silicon dioxide particles are mixed at from 1000 to 2000 revolutions per minute (rpm). In embodiments, the high-shear mixer is a grinder. In embodiments, provided herein is a method of making a tablet comprising compressing a Cannabinoid Product described herein in a tablet press. In embodiments, the tablet is about 100 to 800 mg. In embodiments, the tablet comprises from about 0.1% to about 30% of cannabinoid oil by weight. In embodiments, the tablet is an orodispersable tablet. In embodiments, provided herein is a method of making a Cannabinoid Product described herein comprising: (a) mixing the cannabinoid oil and colloidal silicon dioxide particles; and (b) adding the lubricant. In embodiments, the method further comprises (c) adding a filler. In embodiments, the filler is silicified microcrystalline cellulose. In embodiments, the cannabinoid oil is diluted in a solvent before mixing with colloidal silicon dioxide particles. In embodiments, the solvent is ethanol. In embodiments, step (a) is performed before step (b). In embodiments, step (a) is performed before step (b), and step (b) is performed before step (c). In embodiments, step (a) is performed before step (c), and step (c) is performed before step (b). In embodiments, the method comprises heating the cannabinoid oil before the oil is mixed with the colloidal silicon dioxide particles. In embodiments, the cannabinoid oil and colloidal silicon dioxide particles are heated after mixing. In embodiments, the method comprises heating the cannabinoid oil to at least 30° C., at least 40° C., at least 50° C., at least 60° C., at least 70° C., at least 80° C., or at least 90° C. In embodiments, the method comprises adding the cannabinoid oil to the colloidal silicon dioxide particles. In embodiments, the method comprises adding the colloidal silicon dioxide particles to the cannabinoid distillate. In embodiments, the method comprises mixing the cannabinoid oil and colloidal silicon dioxide particles in a high-shear mixer. In embodiments, the method comprises mixing the cannabinoid oil and colloidal silicon dioxide particles in a high-shear mixer for at least 30 seconds, at least 1 minute, at least 5 minutes, at least 10 minutes, at least 15 minutes, or at least 20 minutes. In embodiments, the method comprises mixing the cannabinoid oil and colloidal silicon dioxide particles at a speed of from about 1000 revolutions per minute (rpm) to about 2000 rpm. In embodiments, the method comprises mixing the lubricant with a mixture containing cannabinoid oil, colloidal silicon dioxide particles, filler, and disintegrant for from 1 minute (min) to about 2 min. In embodiments, the high-shear mixer is a grinder. In embodiments, provided herein is a capsule comprising filling a capsule shell with a Cannabinoid Product described herein. In embodiments, the capsule shell comprises gelatin or hydroxypropylmethylcellulose. In embodiments, the capsule comprises from about 1% to about 50% cannabinoid oil by weight.

### BRIEF DESCRIPTION OF THE DRAWINGS

FIG. 1A shows a composition containing a cannabinoid distillate. The composition does not contain colloidal silicon dioxide particles. FIG. 1B shows a composition containing a cannabinoid distillate and colloidal silicon dioxide particles.

FIG. 2 shows an image of the Torpac ProFiller® 3700 capsule filler used to fill the capsules of Examples 1, 2, 5, and 6.

FIG. 3 shows a scanning electron micrograph of colloidal silicon dioxide particles of the disclosure. (Technical Information 1414 AEROPERL® 300 Pharma Improving the Dissolution of Poorly Soluble APIs. EVONIK.).

FIGS. 4A-4B depicts a machine used to determine the hardness of a tablet. FIG. 4A shows an image of the top of the machine, and FIG. 4B shows an image of the machine from the side.

FIG. 5 is a bar graph showing the median percent change from baseline numerical ratings from the clinical study described in Example 8.

### DETAILED DESCRIPTION

### I. Definitions

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Throughout this application, the term “about” is used to indicate that a value includes the inherent variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

Herein, the terms “comprise”, or variations such as “comprises” or “comprising”, will be understood to imply the inclusion of a stated element or integer or group of elements or integers but not the exclusion of any other element or integer or group of elements or integers.

The term “substantially pure” refers to a cannabinoid having a chromatographic purity of greater than about 95% by weight, greater than about 96% by weight, greater than about 97% by weight, greater than about 98% by weight, or greater than about 99% by weight.

Various concentration expressions, including volume concentrations, weight concentrations, and mass concentrations, are utilized to describe the percentage of a component in a solution. Volume concentration has units of % v/v, where v/v is volume per volume. If a solution contains 5% v/v of a component, 5 mL of the component is in a total solution of 100 mL, for example. Weight concentration of a solution is expressed as % w/w, where w/w is weight per weight. If a solution contains 30% w/w of sodium chloride, an example solution contains 30 g of sodium chloride and 70 g of other components of the solution, such as the solvent. Mass concentration of a solution is expressed as % w/v, where w/v is weight per volume. If 1 g of sodium chloride is dissolved in a solution with a total volume of 100 mL, a 1% w/v sodium chloride solution has been made.

The term “Cannabis plant part” refers to any part of a Cannabis plant including but not limited to the embryo, shoot, root, stem, seed, stipule, leaf, petal, flower, inflorescence, bud, ovule, bract, trichome, branch, petiole, internode, bark, pubescence, tiller, rhizome, frond, blade, pollen, stamen, mesocarp, epicarp, endosperm, spermoderm, and disk.

The term “Cannabinoid Product” refers to a composition comprising chemical constituents of Cannabis plants. In embodiments, the Cannabinoid Product is a powder.

The term “cannabinoid oil,” used interchangeably with “cannabinoid” refers to a composition containing one or more cannabinoids. In some embodiments, the cannabinoid oil is a viscous liquid, and has not fully crystallized. In some embodiments cannabinoid oil is produced by extracting cannabinoids from a Cannabis plant or Cannabis plant part(s). In other embodiments cannabinoid oil is produced synthetically or biosynthetically.

The term “cannabinoid distillate” refers to a liquid cannabinoid oil composition that has been distilled. Distillates may contain more than one cannabinoid and may contain other cannabis compounds, such as terpenes. A cannabinoid distillate is substantially free of waxes and chlorophylls, but remains in liquid, oily form.

The term “cannabinoid isolate” refers to a composition comprising at least 95% of a single cannabinoid that is typically solid at room temperature. Cannabinoid isolate is typically in crystalline form at room temperature, or rapidly crystallizes when exposed to nucleation factors, such as other powders. Cannabinoid isolate can be made into a liquid by heating the isolate for a sufficient period of time to melt it.

The term “substantially disintegrates” refers to a level of disintegration amounting to disintegration of at least about 50%, at least about 60%, at least about 70%, at least about 80%, at least about 90%, or about 100% disintegration. The term “disintegration” is distinguished from the term “dissolution,” in that “disintegration” refers to the breaking up of or loss of structural cohesion of e.g. the constituent particles comprising a tablet, whereas “dissolution” refers to the solubilization of a solid in a liquid (e.g., the solubilization of a Cannabinoid Product in solvents or gastric fluids). Disintegration may be measured by recording the amount of time it takes for a tablet of a particular size to disintegrate. Alternatively, the rate of disintegration can be measured using various in vitro test methods, for example the United States Pharmacopeia (USP)<701> Disintegration Test. USP <701> is incorporated by reference herein in its entirety. Dissolution may be measured using a USP Type II paddle dissolution apparatus (paddles at 100 rpm) or a USP Type I basket apparatus at a temperature of about 37° C. at a particular time. The percentage of Cannabinoid Product dissolved at 60 minutes is referred to as the “Percentage Dissolved at 60 minutes.” In embodiments, dissolution measurements use a high performance liquid chromatography, ultraviolet visible spectroscopy, and fiber optic dissolution may be used to measure dissolution.

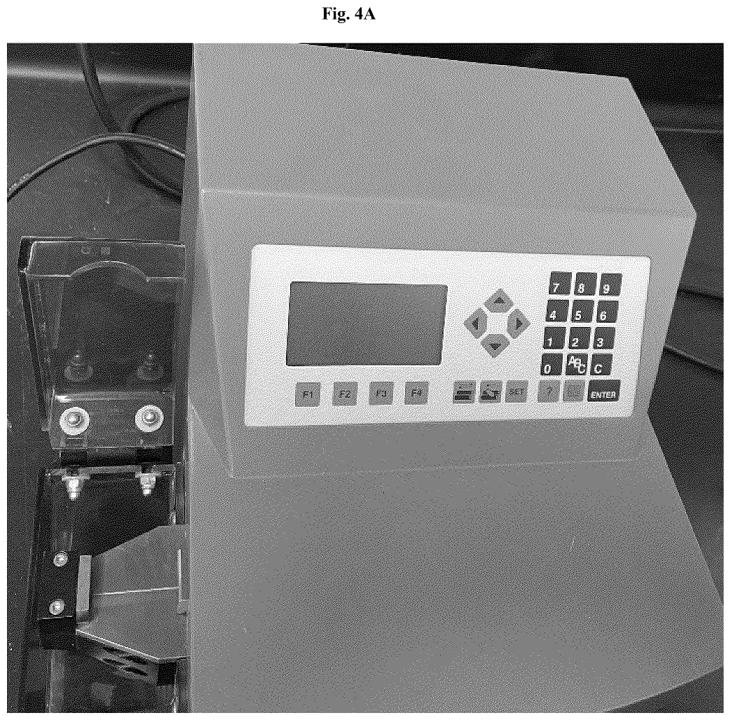

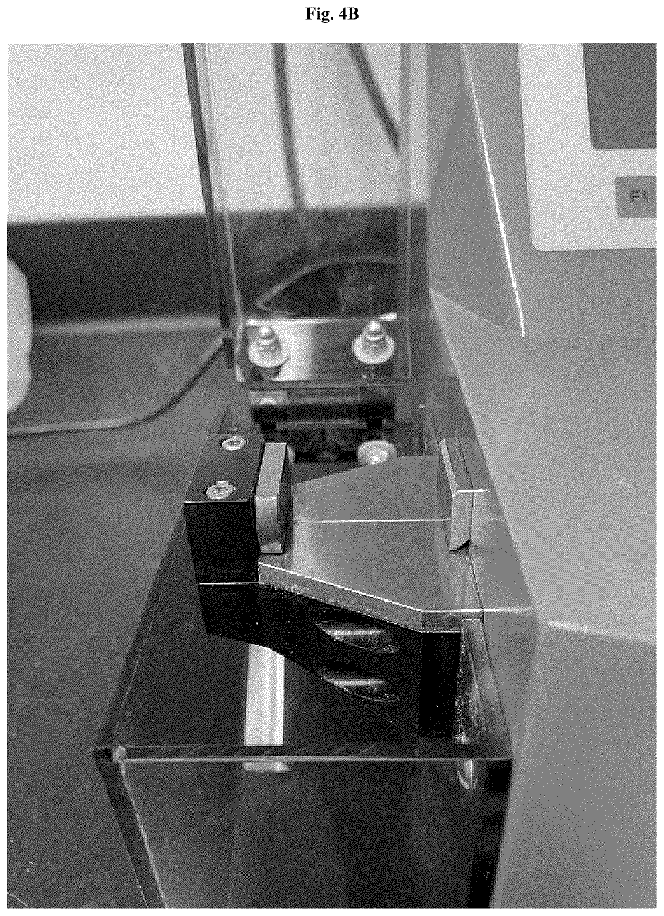

The term “hardness” refers to a tablet's “breaking force,” which is the force required to cause the tablet to fail in a specific plane. A tablet's “breaking force” is measured by placing the tablet between two platens, one of which moves to apply sufficient force to cause fracture. For round tablets, fracture occurs in the plane of loading, which occurs across the tablet's diameter. USP <1217> describes the tablet breaking force and is incorporated by reference herein in its entirety. FIG. 4 depicts a machine used to determine the hardness of a tablet.

The term “compression force” refers to the force exerted between the upper and lower punches when compressing a Cannabinoid Product into a tablet as determined by a load cell transducer that converts the force into electrical signal using a strain gauge. The compression force may be measured in Newtons (N).

The terms “orally disintegrating tablet,” “orally dispersing tablet,” or “ODT” refer to a solid dosage form of the present invention, which disintegrates rapidly in the oral cavity of a patient after administration, without chewing.

### II. Cannabis

In some embodiments, the pharmaceutical of the present disclosure is a Cannabinoid Product. Cannabinoid Products contain chemical constituents from Cannabis plants, or alternatively equivalent products derived from chemical or other biosynthetic means. Cannabis is a genus of flowering plants that includes three different species, Cannabis sativa, Cannabis indica and Cannabis ruderalis. There are 483 identifiable chemical constituents known to exist in the cannabis plant (Rudolf Brenneisen (2007) in Marijuana and the Cannabinoids, ElSohly, ed.; incorporated herein by reference), including at least 85 different cannabinoids and over 120 terpenes (El-Alfy, Abir T, et al. (2010) Pharmacology Biochemistry and Behavior 95 (4): 434-42; incorporated herein by reference). The two most well-known cannabinoids produced by Cannabis plants are tetrahydrocannabinol (THC) and cannabidiol (CBD).

### II-A. Cannabinoids

Cannabinoids are a unique family of terpeno-phenolic compounds produced by Cannabis plants. Typical cannabinoids isolated from Cannabis plants include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C4 (THCA-C4), Tetrahydrocannabinol C4(THC-C4), Tetrahydrocannabivarinic acid (THCVA), Tetrahydrocannabivarin (THCV), Tetrahydrocannabinolic acid-C4(THCA-C4), Tetrahydrocannabinol-C1 (THC-C1), Δ7-cis-iso-tetrahydrocannabivarin, Δ8-tetrahydrocannabinolic acid (Δ8-THCA), Cannabivarinodiolic (CBNDVA), Cannabivarinodiol (CBNDV), Δ8-tetrahydrocannabinol (Δ8-THC), Δ9-tetrahydrocannabinol (Δ9-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA), Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodiolic Acid (CBNDA), Cannabinodivarin (CBDV), Cannabitriol (CBT), 10-Ethoxy-9-hydroxy-Δ6a-tetrahydrocannabinol, 8,9-Dihydroxy-Δ6a(10a)-tetrahydrocannabinol (8,9-Di-OH-CBT-C5), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, and Dodeca-2E,4E-dienoic acid isobutylamide. See Holley et al. (1975) J. Pharm. Sci. 64:892-894 and De Zeeuw et al. (9172) Science 175:778-779, each of which is herein incorporated by reference in its entirety for all purposes.

Most cannabinoids exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). Cannabinoids in their acid forms (those ending in “-A”) can be converted to their non-acidic forms through a process called decarboxylation. While some decarboxylation (e.g., neutralization) of cannabinoids does occur in the plant, production of the neutral forms increases significantly post-harvest. (Sanchez and Verpoorte (2008) Plant Cell Physiol. December: 49(12)). Full decarboxylation of phytocannabinoids can be catalyzed by post-cultivation heating cannabis plant material or extracted cannabinoids (e.g., by combustion, vaporization, or baking in an oven).

In order to find the total amount of non-acidic cannabinoid, the total measured content of acid cannabinoid variants forms should be adjusted to account for the loss of the carboxyl group. In some embodiments, this adjustment can be made by multiplying the molar content of the acidic cannabinoid forms by the molecular weight of the corresponding decarboxylated cannabinoid. Other shorthand conversions are also available for quickly converting acidic cannabinoid content to active cannabinoid content.

For example, in some embodiments, THCA can be converted to active THC using the formula: THCA×0.877=THC. When using this approach, the maximum THC for the sample is: THCmax=(THCA×0.877)+THC. This method has been validated according to the principles of the International Conference on Harmonization. Similarly, CBDA can be converted to active CBD and the yield is determined using the yield formula: CBDA×0.877=CBD. Also, the maximum amount of CBD yielded, i.e. max CBD for the sample is: CBDmax=(CBDA×0.877)+CBD. Additionally, CBGA can be converted to active CBG by multiplying CBGA by 0.878 (CBGmax=(CBGA×0.878)+CBG).

In some embodiments, provided herein are Cannabinoid Products containing cannabinoids. In some embodiments, the Cannabinoid Products contain a cannabinoid selected from the group consisting of: cannabidiol (CBD), tetrahydrocannabinol (THC), delta 8 tetrahydrocannabinol (D8 THC or Δ8 THC), tetrahydrocannabivarin (THCV), cannabidiolic acid (CBDA), cannabidivarin (CBDV), tetrahydrocannabinolic acid (THCA), cannabigerol (CBG), cannabigerolic acid (CBGA), cannabinol (CBN), cannabinolic acid (CBNA), cannabidivarinic acid (CBDVA), cannabichromenic acid (CBCA), and cannabichromene (CBC).

Brief descriptions and chemical structures of the aforementioned cannabinoids are provided below.

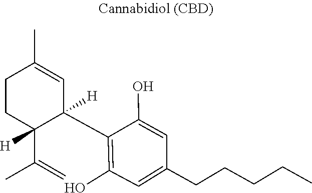 [SMILES: C=C(C)[C@@H]1CCC(C)=C[C@H]1c1c(O)cc(CCCCC)cc1O]

CBD is a cannabinoid found in Cannabis shown to display sedative effects in animal tests (Pickens, (1981) Br. J. Pharmacol. 72 (4): 649-56). Some research, however, indicates that CBD can increase alertness, and attenuates the memory-impairing effect of THC. (Nicholson et al., June (2004) J Clin Psychopharmacol 24 (3): 305-13; Morgan et al., (2010) The British Journal of Psychiatry, 197:258-290). CBD may also decrease the rate of THC clearance from the body, perhaps by interfering with the metabolism of THC in the liver. Medically, CBD has been shown to relieve convulsion, inflammation, anxiety, and nausea, as well as inhibit cancer cell growth (Mechoulam, et al., 2007, Chemistry & Biodiversity 4 (8): 1678-1692), for example reducing growth and invasiveness of aggressive human breast cancer cells (McAllister et al., 2007, Mol. Cancer Ther. 6 (11): 2921-7) Recent studies have also shown CBD to be as effective as an atypical antipsychotic for treating schizophrenia (Zuardi et al., 2006, Braz. J. Med. Biol. Res. 39 (4): 421-429.), and studies also suggests that CBD may relieve symptoms of dystonia (Consroe, 1986, The International journal of neuroscience 30 (4): 277-282).

Cannabis produces cannabidiolic acid (CBDA) through the same metabolic pathway as THC, until the last step, where CBDA synthase performs catalysis instead of THCA synthase. See Marks et al. (2009) Journal of Experimental Botany 60 (13): 3715-3726 and Meijer et al. I, II, III, and IV.

Non-limiting examples of CBD variants include CBD-C5, CBDM-C5, CBD-C4, CBDV-C3, CBD-C1, CBDA-C5, and CBDVA-C3.

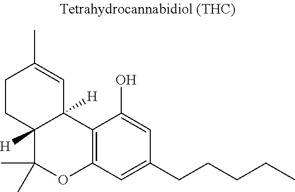 [SMILES: CCCCCc1cc(O)c2c(c1)OC(C)(C)[C@@H]1CCC(C)=C[C@@H]21]

Unless indicated otherwise, as used herein, THC refers to delta-9-tetrahydrocannabinol (Δ9-THC) (structure above). THC is the principal psychoactive constituent (or cannabinoid) of the Cannabis plant. THC has mild to moderate analgesic effects, and Cannabis can be used to treat pain by altering transmitter release on dorsal root ganglion of the spinal cord and in the periaqueductal gray. Other effects include relaxation, alteration of visual, auditory, and olfactory senses, fatigue, and appetite stimulation. THC has marked antiemetic properties, and may also reduce aggression in certain subjects (Hoaken (2003) Addictive Behaviors 28: 1533-1554).

The pharmacological actions of THC result from its partial agonist activity at the cannabinoid receptor CB1, located mainly in the central nervous system, and the CB2 receptor, mainly expressed in cells of the immune system (Pertwee, (2006) International Journal of Obesity 30: S13-S18.) It is also suggested that THC has an anticholinesterase action, which may implicate it as a potential treatment for Alzheimer's and Myasthenia gravis (Eubanks et al., (2006) Molecular Pharmaceutics 3 (6): 773-7).

In the cannabis plant, THC occurs mainly as tetrahydrocannabinolic acid (THCA, 2-COOH-THC). Geranyl pyrophosphate and olivetolic acid react via an enzyme-catalyzed reaction to produce cannabigerolic acid, which is cyclized by the enzyme THC acid synthase to give THCA. Over time, or when heated, THCA is decarboxylated producing THC.

Non-limiting examples of THC variants include Δ9-THC-C5, Δ9-THC-C4, Δ9-THCV-C3, Δ9-THCO-C1, Δ9-THCA-C5 A, Δ9-THCA-C5 B, Δ9-THCA-C4 A, Δ9-THCA-C4 B, Δ9-THCVA-C3 A, Δ9-THCOA-C1 A, Δ9-THCOA-C1 B, Δ8-THC-C5, Δ8-THCA-C5 A, (−)-cis-Δ9-THC-C5. The structure of Δ8-THC is below.

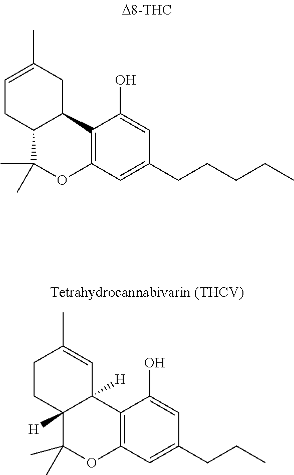

THCV is a propyl analogue of tetrahydrocannabinol. Although THCV possesses an almost identical structure to Δ9-THC (varying only by the length of its lipophilic alkyl chain), it has different molecular targets and pharmacological profile. Compared to THC which demonstrates its effects through weak partial agonist activity of both endocannabinoid receptors Cannabinoid-1 (CB1R) and Cannabinoid-2 (CB2R), THCV acts as a CB1 antagonist and a partial agonist of CB2. THCV has been reported to activate 5HT1A receptors to produce an antipsychotic effect that has therapeutic potential for ameliorating some of the negative, cognitive and positive symptoms of schizophrenia (Br J Pharmacol. 2015 March; 172(5): 1305-1318.) THCV has also shown antiepileptiform and anticonvulsant properties that suggest possible therapeutic application in the treatment of pathophysiologic hyperexcitability states such as untreatable epilepsy (Epilepsia. 2010 August; 51(8):1522-32.).

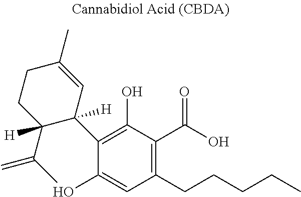 [SMILES: C=C(C)[C@@H]1CCC(C)=C[C@H]1c1c(O)cc(CCCCC)c(C(=O)O)c1O]

CBDA is most abundant in the glandular trichromes on Cannabis buds. CBDA is the chemical precursor to CBD. CBDA has been shown to exert therapeutic effects, including antiemetic, anti-inflammatory, anxiolytic, and antidepressant effects. (Behav Pharmacol. 2020 September; 31(6):591-596.) An in vivo rodent study showed that CBDA reduces thermal pain sensitivity in a model of Rett syndrome. (Neuroscience. 2021 Jan. 15; 453, 113-123.)

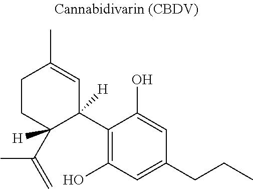 [SMILES: C=C(C)[C@@H]1CCC(C)=C[C@H]1c1c(O)cc(CCC)cc1O]

CBDV is a non-psychoactive cannabinoid found in Cannabis. It is a homolog of CBD with the side-chain shortened by two methylene bridges (CH₂ units). CBDV has demonstrated anticonvulsant activity in animal models and in human clinical trials. (Br J Pharmacol. 2013 October; 170(3):679-92.) CBDV also acts as a functional partial agonist on dopamine D2-like receptors in vivo, supporting its therapeutic use in neurological disorders. (Biochemical and Biophysical Research Communications. 2020; 533(4):1366-1370.).

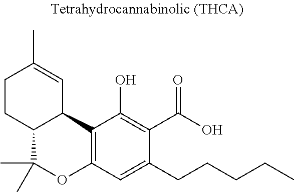 [SMILES: CCCCCc1cc2c(c(O)c1C(=O)O)[C@@H]1C=C(C)CC[C@H]1C(C)(C)O2]

THCA is found in variable quantities in fresh, undried Cannabis, but is progressively decarboxylated to THC with drying and heating. THCA is produced from cannabigerolic acid by THCA synthase. THCA has been shown to have anti-inflammatory properties. (Biol Pharm Bull. 2011; 34(5):774-8.).

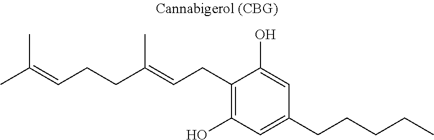 [SMILES: CCCCCc1cc(O)c(C/C=C(\C)CCC=C(C)C)c(O)c1]

CBG is a non-psychoactive cannabinoid found in the Cannabis plants. CBG has been found to act as a high affinity α2-adrenergic receptor agonist, moderate affinity 5-HT1A receptor antagonist, and low affinity CB1 receptor antagonist. It also binds to the CB2 receptor. Cannabigerol has also been shown to reduce depression in animal models (US Patent Application Publication No. 2008-0031977). In particular, CBG has been shown to have significant potential applications in the treatment of glaucoma, depression, Huntington's disease, MRSA, cachexia, and cancer (Craig et al. 1984, Experimental eye research 39 (3):251-259; U.S. Pat. No. 8,481,085; Valdeolivas et al. 2015, Neurotherapeutics January 12(1):185-99; Appendino G et al., 2008, J. Nat Prod. August: 71(8):1427-30; Borrelli F et al. 2013, Biochem Pharmacol May 1:85(9):1306-16; Borrelli F. et al. 2014, Carcinogenesis December:35(12):2787-97) Non-limiting examples of CBG variants include (E)-CBG-C5, (E)-CBGM-C5 A, (Z)-CBGA-C5 A, (E)-CBGV-C3, (E)-CBGA-C5 A, (E)-CBGAM-C5 A, and (E)-CBGVA-C3 A.

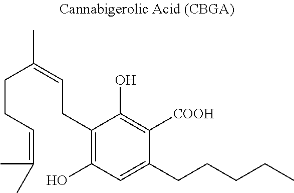 [SMILES: CCCCCc1cc(O)c(C/C=C(/C)CCC=C(C)C)c(O)c1C(=O)O]

CBGA is the acidic form of CBG. CBGA is synthesized from olivetolic acid and geranyl diphosphate. CBGA is a precursor to THCA, CBDA, and CBCA. CBGA has been found to inhibit aldose reductase activity, suggesting that it may have therapeutic value for treatment of complications of diabetes. (Fitoterapia. 2018 June; 127:101-108.).

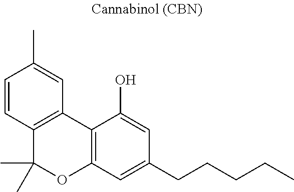 [SMILES: CCCCCc1cc(O)c2c(c1)OC(C)(C)c1ccc(C)cc1-2]

CBN is a mildly psychoactive cannabinoid found in trace amounts in Cannabis. CBN is a metabolite of THC. CBN has been shown to have significant applications in the treatment of anxiety disorder, insomnia, inflammation, convulsions, and bacterial infections. (Herring et al. Journal of Pharmacology and Experimental Therapeutics December 1999, 291 (3) 1156-1163.) CBN may be produced by aromatizing THC.

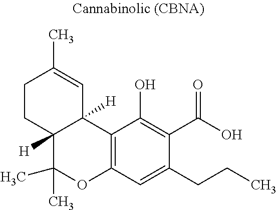 [SMILES: CCCc1cc2c(c(O)c1C(=O)O)[C@@H]1C=C(C)CC[C@H]1C(C)(C)O2]

CBNA is found in trace amounts in Cannabis. CBNA is produced from THCA after exposure to ultraviolet light. CBNA has therapeutic benefit for treatment of insomnia, convulsions, and chronic pain.

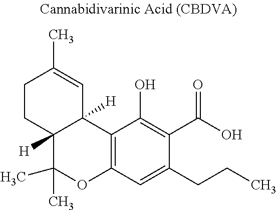 [SMILES: CCCc1cc2c(c(O)c1C(=O)O)[C@@H]1C=C(C)CC[C@H]1C(C)(C)O2]

CBDVA is the acidic form of CBDV. Extracts containing CBDVA exhibit antioxidant and antimicrobial activity against methicillin-resistant strains of Staphylococcus aureus. (Phytother Res. 2021 February; 35(2):1099-1112.).

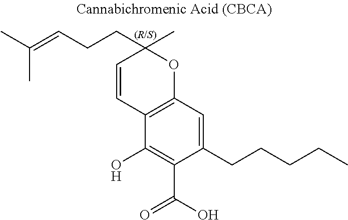 [SMILES: CCCCCc1cc2c(c(O)c1C(=O)O)C=CC(C)(CCC=C(C)C)O2]

CBCA is the acidic form of CBC. CBCA is produced from CBGA. Geranyl pyrophosphate and olivetolic acid combine to produce CBGA, which is cyclized by the enzyme CBCA synthase to form CBCA. In vitro studies show that CBCA has more potent bactericidal activity than vancomycin, the currently recommended treatment for methicillin-resistant Staphylococcus aureus infections. (Antibiotics 2020, 9(8), 523).

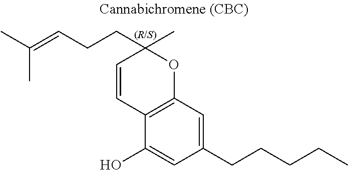 [SMILES: CCCCCc1cc(O)c2c(c1)OC(C)(CCC=C(C)C)C=C2]

CBC is produced from decarboxylation of CBCA. CBC is an agonist of TRPA1 and less potently an agonist of TRPV3 and TRPV4. CBC inhibits nitric oxide production in macrophages and ameliorates murine colitis. (Br J Pharmacol. 2013 May; 169(1): 213-229; Acta Physiol (Oxf). 2012 February; 204(2):255-66.).

### II-B. Additional Components of Cannabis

In embodiments, in addition to containing cannabinoids, the Cannabinoid Products described herein may comprise additional components present in Cannabis, and other plants. Examples of additional components of Cannabis include non-specific lipid-soluble material or “ballast” e.g. waxes, wax esters and glycerides, unsaturated fatty acid residues, terpenes, chlorophyll, carotenes, flavonoids, pigments, sugars, cellulose compounds, and minerals.

Cannabis produces over 120 different terpenes (Russo (2011) British Journal of Pharmacology, 163:1344-1364). Within the context and verbiage of this document the terms ‘terpenoid’ and ‘terpene’ are used interchangeably. In some embodiments, the present disclosure provides compositions comprising one or more terpenes or terpenoids.

In addition to many circulatory and muscular effects, some terpenes interact with neurological receptors. A few terpenes produced by cannabis plants also bind weakly to cannabinoid receptors. Some terpenes can alter the permeability of cell membranes and allow in either more or less THC, while other terpenes can affect serotonin and dopamine chemistry as neurotransmitters. Terpenoids are lipophilic, and can interact with lipid membranes, ion channels, a variety of different receptors (including both G-protein coupled odorant and neurotransmitter receptors), and enzymes. Some are capable of absorption through human skin and passing the blood brain barrier.

Terpenoids are mainly synthesized in two metabolic pathways: mevalonic acid pathway (a.k.a. HMG-CoA reductase pathway, which takes place in the cytosol) and MEP/DOXP pathway (a.k.a. The 2-C-methyl-D-erythritol 4-phosphate/1-deoxy-D-xylulose 5-phosphate pathway, non-mevalonate pathway, or mevalonic acid-independent pathway, which takes place in plastids). Geranyl pyrophosphate (GPP), which is used by cannabis plants to produce cannabinoids, is formed by condensation of dimethylallyl pyrophosphate (DMAPP) and isopentenyl pyrophosphate (IPP) via the catalysis of GPP synthase. Alternatively, DMAPP and IPP are ligated by FPP synthase to produce farnesyl pyrophosphate (FPP), which can be used to produce sesquiterpenoids. Geranyl pyrophosphate (GPP) can also be converted into monoterpenoids by limonene synthase.

Terpenes are derived biosynthetically from units of isoprene, which have the molecular formula CSHs. The basic molecular formulae of terpenes are multiples of (C₅H₈)ₙ where n is the number of linked isoprene units. The isoprene units may be linked together “head to tail” to form linear chains or they may be arranged to form rings.

Within the context of this disclosure, the term “terpene” includes Hemiterpenes, Monoterpenols, Terpene esters, Diterpenes, Monoterpenes, Polyterpenes, Triterpenes, Tetraterpenes, Terpenoid oxides, Sesterterpenes, Sesquiterpenes, Norisoprenoids, as well as their isomers, enantiomers, or derivatives. Within the context of this disclosure, the term terpene includes the α-(alpha), β-(beta), γ-(gamma), oxo-, isomers, or any combinations thereof.

In some embodiments, the present disclosure provides Cannabinoid Products containing terpenes. Non-limiting examples of terpenes include: Alpha Pinene, Limonene, Beta Pinene, Alpha Phellandrene, Terpinolene, Nerolidol, Nerol, Myrcene, Beta Caryophyllene, 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10), 11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, and Vanillin.

In some embodiments, the Cannabinoid Products described herein comprise a derivative of a terpene. Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids. Terpenoids, a.k.a. isoprenoids, are a large and diverse class of naturally occurring organic chemicals similar to terpenes, derived from five-carbon isoprene units assembled and modified in thousands of ways. Non-limiting examples of terpenoids include, Hemiterpenoids, 1 isoprene unit (5 carbons); Monoterpenoids, 2 isoprene units (10C); Sesquiterpenoids, 3 isoprene units (15C); Diterpenoids, 4 isoprene units (20C) (e.g. ginkgolides); Sesterterpenoids, 5 isoprene units (25C); Triterpenoids, 6 isoprene units (30C) (e.g. sterols); Tetraterpenoids, 8 isoprene units (40C) (e.g. carotenoids); and Polyterpenoid with a larger number of isoprene units.

### III. Cannabinoid Products

This disclosure provides Cannabinoid Products and oral pharmaceutical compositions (e.g., tablets and capsules) comprising the same. The Cannabinoid Products described herein comprise colloidal silicon dioxide particles, which contain pores, and cannabinoid oils. The initial step of creating the Cannabinoid Products described herein comprises mixing the colloidal silicon dioxide particles and cannabinoid oils. Without being bound by theory, this initial mixing step allows for incorporation of the cannabinoid oil within the pores of the colloidal silicon dioxide particles. As a result, the cannabinoid oil does not separate from the powder ingredients of the Cannabinoid Product. Thus, the Cannabinoid Products described herein are homogenous free-flowing powders. Although, colloidal silicon dioxide particles traditionally cause difficulties with tablet compression, Applicants have developed novel formulations for Cannabinoid Products and methods of making the same that provide for compressible tablets.

### III-A. Cannabinoid Oils

In embodiments, the Cannabinoid Products comprise cannabinoid oils. In some embodiments, the cannabinoid oil refers to a composition produced by extracting cannabinoids from a Cannabis plant or Cannabis plant part(s).

In embodiments, a cannabinoid oil is produced using any suitable extraction method. In embodiments, the extraction method is selected from the group consisting of maceration, percolation, solvent extraction, steam distillation, and vaporization. General protocols for the preparation of cannabinoid oils are described in the following patent documents: U.S. Pat. Nos. 8,603,515; 9,730,911; 7,700,368, 10,159,908, U.S. Pub. No. 2019/0151771, U.S. Pub. No. 2018/0078874, U.S. Pub. No. 2020/0080021, U.S. Pub. No. 2020/0048214, U.S. Pub. No. 2020/0048215, and U.S. patent Ser. No. 10/555,914. Each of the aforementioned patent documents are incorporated by reference herein in their entireties.

Solvent extraction may be carried out using essentially any solvent that dissolves cannabinoids/cannabinoid acids, such as for example C1 to C5 alcohols (e.g. ethanol, methanol), C4-C12 alkanes (e.g. hexane or butane), Norflurane (HFA134a), HFA227, and carbon dioxide. When solvents such as those listed above are used, the resultant primary extract typically contains non-specific lipid-soluble material or “ballast” e.g. waxes, wax esters and glycerides, unsaturated fatty acid residues, terpenes, carotenes, and flavonoids. The primary extract may be further purified for example by “winterization”, which involves chilling to −20° C. followed by filtration to remove waxy ballast, supercritical or subcritical extraction, vaporization, distillation, and chromatography.

In some embodiments, the cannabinoid oil may be obtained by carbon dioxide (CO2) extraction followed by a secondary extraction, e.g. an ethanolic precipitation, to remove a substantial proportion of non-cannabinoid materials. In some embodiments, a cannabinoid oil is produced by a process comprising extraction with liquid CO₂ under sub-critical or super-critical conditions, and then a further extraction (e.g., an ethanolic precipitation) to remove significant amounts of ballast. If it is intended to prepare free cannabinoids from the Cannabis plant parts, then the plant parts preferably heated to a defined temperature for a defined period of time in order to decarboxylate cannabinoid acids to free cannabinoids prior to extraction of the botanical drug substance.

In some embodiments, a cannabinoid oil is prepared according to a process comprising the following steps: i) optional decarboxylation of the plant material, ii) extraction with liquid CO₂ (in some embodiments under sub-critical conditions), to produce a crude botanical drug substance, iii) precipitation with C1-C5 alcohol to reduce the proportion of non-target materials, iv) removal of the precipitate (preferably by filtration), v) optional treatment with activated charcoal, and vi) evaporation to remove C1-C5 alcohol and water, thereby producing a final botanical drug substance.

In some embodiments, a cannabinoid oil is prepared from plant parts according to a process comprising the following steps: i) CO2 extraction for plant terpenes, ii) ethanol extraction for crude cannabinoids, plant waxes, and plant oils (crude extract); iii) winterization of the crude extract at −80° C. for 24 hours; and iv) complete ethanol recovery and in-vessel decarboxylation of winterized crude before fractional distillation of cannabinoids.

In some embodiments, a cannabinoid oil described herein is extracted from Cannabis using any method known in the art and subsequently winterized.

In embodiments, the cannabinoid oil is a cannabinoid distillate. In embodiments, the cannabinoid distillate comprises from 80% to 90% cannabidiol (CBD) and less than 0.1% delta-9-tetrahydrocannabinol by weight. In embodiments, the cannabinoid distillate comprises from 80% to 90% cannabidiol (CBD) and less than 0.3% delta-9-tetrahydrocannabinol by weight. In embodiments, the distillate comprises cannabigerol (CBG), cannabinol (CBN), or a combination thereof.

In embodiments, the cannabinoid comprises up to 50% CBD and up to 30% other cannabinoids by weight. In embodiments, the cannabinoid does not form crystals. In embodiments, the cannabinoid comprises CBD, CBG, CBN, and cannabichromene (CBC).

In embodiments, the cannabinoid distillate comprises 70-80% CBN and 8-15% other cannabinoids by weight (i.e., CBC, CBG, CBD, cannabidivarin (CBDV), cannabicitran (CBT), and combinations thereof).

In embodiments, the cannabinoid distillate comprises 70-85% CBG and 8-15% other cannabinoids (i.e., CBC, CBD, CBN, CBDV, CBT, and combinations thereof) by weight. In embodiments, the distillate contains about 25% CBD and up to 15% CBC and CBN by weight.

In embodiments, the cannabinoid distillate comprises from 80% to 92% delta-8-tetrahydrocannabinol. In embodiments, the cannabinoid distillate comprises about 84.8% delta-8-tetrahydrocannabinol by weight. In embodiments, the cannabinoid distillate comprises about 90.5% delta-8-tetrahydrocannabinol by weight.

In embodiments, the cannabinoid distillate comprises about 73.2% THC by weight.

In embodiments, the Cannabinoid Products comprise from about 1% to about 60% cannabinoid oil by weight. In embodiments, the Cannabinoid Products comprise from about 1% to about 50% cannabinoid oil by weight. In embodiments, the Cannabinoid Products comprise from about 1% to about 30% cannabinoid oil by weight. In embodiments, the Cannabinoid Products comprise from about 10% to about 60% cannabinoid oil by weight. For example, in embodiments, the Cannabinoid Products comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% cannabinoid oil by weight of the composition, including all subranges, ranges, and values therebetween. In embodiments, the cannabinoid oil is a cannabinoid distillate.

### III-B. Colloidal Silicon Dioxide Particles

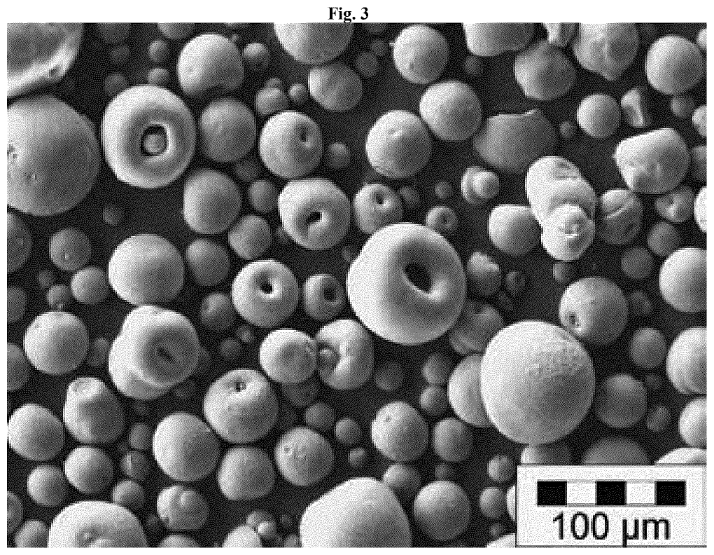

In embodiments, the Cannabinoid Products described herein comprise colloidal silicon dioxide particles. Colloidal silicon dioxide particles are comprised of amorphous synthetic silicon dioxide with pores. FIG. 3 shows an image of colloidal silicon dioxide particles. The following document describes colloidal silicon dioxide particles and is incorporated by reference herein in its entirety: EVONIK®; Technical Information 1414: AEROPERL® 300 Pharma Improving the dissolution of poorly soluble APIs.

In embodiments, the size of colloidal silicon dioxide particles ranges from about 20 μm to about 60 μm, as determined by scanning electron microscopy. The particle size refers to the diameter of the particle. In embodiments, the average particle size of the colloidal silicon dioxide particles is about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, or about 60 μm, including all values, subranges, and ranges therebetween.

In embodiments, the pores of the colloidal silicon dioxide particles have volumes ranging from about 1.5 milliliters (mL)/gram (g) to about 1.9 mL/g. For example, the volume of the particle may be about 1.5 mL/g, about 1.55 mL/g, about 1.6 mL/g, about 1.65 mL/g, about 1.7 mL/g, about 1.75 mL/g, about 1.8 mL/g, about 1.85 mL/g, or about 1.9 mL/g.

In embodiments, the Cannabinoid Products comprise from about 20% to about 90%, from about 25% to about 50%, from about 25% to about 35%, from about 35% to about 45%, or about 29.1% to about 58.2% colloidal silicon dioxide particles by weight. In embodiments, the Cannabinoid Products comprise from about 29.1% to about 58.2% colloidal silicon dioxide particles by weight. In embodiments, the Cannabinoid Products comprise about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% colloidal silicon dioxide particles by weight.

### III-C. Filler

In embodiments, the Cannabinoid Products described herein comprise a filler. Non-limiting examples of fillers include silicon dioxide, titanium dioxide, alumina, talc, kaolin, powdered cellulose, microcrystalline cellulose, urea, sodium chloride, as well as saccharides, or combinations thereof. Any suitable saccharide may be used in a Cannabinoid Product described herein. As used herein, the term “saccharide” includes sugar alcohols, monosaccharides, disaccharides, and oligosaccharides. Exemplary sugar alcohols include, but not limited to, xylitol, mannitol, sorbitol, erythritol, lactitol, pentitol, and hexitol. Exemplary monosaccharides include, but are not limited to, glucose, fructose, aldose and ketose. Exemplary disaccharides include, but are not limited to, sucrose, isomalt, lactose, trehalose, and maltose. Exemplary oligosaccharides include, but are not limited to, fructo-oligosaccharides, inulin, galacto-ologosaccharides, and mannan-oligosaccharides. In some embodiments, the saccharide is sorbitol, mannitol, or xylitol. In some embodiments, the saccharide is sorbitol. In some embodiments, the saccharide is sucrose.

In embodiments, the filler is mannitol. The chemical structure of mannitol is below:

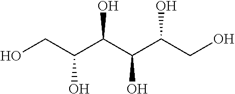 [SMILES: OC[C@@H](O)[C@@H](O)[C@H](O)[C@H](O)CO]

In embodiments, the filler is microcrystalline cellulose. In embodiments, the filler is silicified microcrystalline cellulose. Silicified microcrystalline cellulose is a combination of microcrystalline cellulose and colloidal silicon dioxide in which the colloidal silicon dioxide binds to the microcrystalline cellulose by noncovalent interactions. In embodiments, silicified microcrystalline cellulose has an average particle size as measured by laser diffraction of from about 40 μm to about 150 μm. For example, in embodiments, the average particle size of the silicified microcrystalline cellulose is about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, about 105 μm, about 106 μm, about 107 μm, about 108 μm, about 109 μm, about 110 μm, about 111 μm, about 112 μm, about 113 μm, about 114 μm, about 115 μm, about 116 μm, about 117 μm, about 118 μm, about 119 μm, about 120 μm, about 121 μm, about 122 μm, about 123 μm, about 124 μm, about 125 μm, about 126 μm, about 127 μm, about 128 μm, about 129 μm, about 130 μm, about 131 μm, about 132 μm, about 133 μm, about 134 μm, about 135 μm, about 136 μm, about 137 μm, about 138 μm, about 139 μm, about 140 μm, about 141 μm, about 142 μm, about 143 μm, about 144 μm, about 145 μm, about 146 μm, about 147 μm, about 148 μm, about 149 μm, or about 150 μm, including all subranges, ranges, and values therebetween. In embodiments, the silicified microcrystalline cellulose has an average particle size as measured by laser diffraction of about 65 μm.

In embodiments, the Cannabinoid Product comprises from about 1% to about 60% filler by weight. In embodiments, the Cannabinoid Product comprises from about 20% to about 45% filler by weight. In embodiments, the Cannabinoid Products comprise from about 30% to about 60% filler by weight. In embodiments, the Cannabinoid Products comprise from about 5% to about 60% filler by weight. In embodiments, the Cannabinoid Products comprise from about 12.3% to about 43.4% filler by weight. For example, in embodiments, the Cannabinoid Product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% filler by weight, including any values, subranges, and ranges therebetween. In embodiments, the Cannabinoid Product comprises about 25% filler by weight.

### III-D. Disintegrant

In embodiments, the Cannabinoid Products described herein comprise a disintegrant. Disintegrants are used to facilitate disintegration of the tablet. Non-limiting examples of disintegrants include starches, clays, celluloses, algins, gums, crosslinked polymers, lightly crosslinked polyvinyl pyrrolidone, corn starch, potato starch, maize starch and modified starches, croscarmellose sodium, crospovidone, sodium starch glycolate, and combinations and mixtures thereof. In embodiments, the disintegrant is sodium starch glycolate or croscarmellose sodium.

In embodiments, the Cannabinoid Product comprises from about 0.5% to about 10% disintegrant by weight. In embodiments, the Cannabinoid Product comprises from about 0.5% to about 5% disintegrant by weight. In embodiments, the Cannabinoid Product comprises from about 2% to about 5.5% disintegrant by weight. In embodiments, the Cannabinoid Product comprises from about 2.5% to about 7.5% disintegrant by weight. For example, in embodiments, the Cannabinoid Product comprises about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10, including any values, subranges, and ranges therebetween. In embodiments, the disintegrant is sodium starch glyolate or croscarmellose sodium.

### III-E. Lubricant

In embodiments, the Cannabinoid Products described herein comprise a lubricant. Lubricants are used to facilitate tablet manufacture, promoting powder flow and preventing particle capping (i.e., particle breakage) when pressure is relieved. Non-limiting examples of lubricants include: magnesium stearate, calcium stearate, stearic acid, glyceryl behenate, talc, mineral oil (in PEG), hydrogenated vegetable oil (e.g., comprised of hydrogenated and refined triglycerides of stearic and palmitic acids), and sodium stearyl fumarate. In embodiments, the lubricant is sodium stearyl fumarate.

In embodiments, the Cannabinoid Product comprises from about 0.5% to about 10% lubricant by weight. In embodiments, the Cannabinoid Product comprises from about 0.1% to about 1% lubricant by weight. In embodiments, the Cannabinoid Product comprises from about 1% to about 2.7% lubricant by weight. For example, in embodiments, the Cannabinoid Product comprises about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, or about 10% lubricant by weight.

### III-F. Cannabinoid Isolates

In embodiments, the Cannabinoid Products described herein comprise a cannabinoid isolate. In embodiments, the Cannabinoid Product contains a cannabinoid isolate containing any cannabinoid described herein. In embodiments, the Cannabinoid Product comprises a cannabinoid isolate comprising CBD. In embodiments, the Cannabinoid Product comprises a cannabinoid isolate comprising CBG. In embodiments, the Cannabinoid Product comprises 1, 2, 3, 4, 5, 6, 7, 8, 9, or 10 cannabinoid isolates.

In embodiments, the Cannabinoid Products comprise from about 1% to about 70% cannabinoid isolate by weight. For example, in embodiments, the Cannabinoid Products comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70% cannabinoid isolate by weight.

### III-G. Flavorings

In embodiments, the Cannabinoid Products described herein comprise a flavoring. Non-limiting examples of flavorings include vanilla, citrus, lemon, orange, lime, grapefruit, yazu, sudachi, apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya, spearmint, cinnamon, wintergreen, peppermint, eucalyptus, and anise.

In embodiments, the flavoring is a terpene. Non-limiting examples of terpenes are found in Section II-B of this disclosure. In embodiments, the terpene comprises myrcene, linalool, or a combination thereof.

In embodiments, the Cannabinoid Products comprise from about 0.01% and about 5% flavorings by weight. For example, the Cannabinoid Products may comprise about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, or about 5% flavorings by weight. In embodiments, the Cannabinoid Products comprise about 0.2% flavoring by weight. In embodiments, the Cannabinoid Products comprise about 0.4% flavoring by weight.

### III-H. Exemplary Cannabinoid Products for Tablets

In embodiments, provided herein are Cannabinoid Products for tablets. In embodiments, a Cannabinoid Product for a tablet comprises: (a) a cannabinoid oil; (b) colloidal silicon dioxide particles; (c) a filler; (d) a disintegrant; and (e) a lubricant; optionally wherein the lubricant is sodium stearyl fumarate; optionally wherein the filler comprises silicified microcrystalline cellulose, mannitol, or combinatons thereof; optionally wherein the disintegrant comprises sodium starch glycolate, croscarmellose sodium, or a combination thereof.

In embodiments, the Cannabinoid Product comprises from about 10% to about 70% of cannabinoid oil by weight. In embodiments, the Cannabinoid Product comprises from about 10% to about 60% of cannabinoid oil by weight. In embodiments, the Cannabinoid Product comprises from about 19.4% to about 41.9% of cannabinoid oil by weight. In embodiments, the Cannabinoid Product comprises from about 1% to about 30% of cannabinoid oil by weight. For example, in embodiments, the Cannabinoid Product comprises about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70% cannabinoid oil by weight, including any values, subranges, and ranges therebetween. In embodiments, the Cannabinoid Product comprises 41.9% cannabinoid oil by weight. In embodiments, the Cannabinoid Product comprises 22.1% cannabinoid oil by weight. In embodiments, the Cannabinoid Product comprises 19.4% cannabinoid oil by weight.

In embodiments, the Cannabinoid Product comprises about 5% to about 60% filler by weight. In embodiments, the Cannabinoid Product comprises about 5% to about 30% filler by weight. In embodiments, the Cannabinoid Product comprises about 30% to about 60% filler by weight. In embodiments, the Cannabinoid Product comprises about 12.3% to about 43.4% filler by weight. For example, in embodiments, the Cannabinoid Product comprises about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% filler by weight, including any values, subranges, and ranges therebetween. In embodiments, the Cannabinoid Product comprises about 46.6% filler by weight. In embodiments, the Cannabinoid Product comprises about 12.3% filler by weight. In embodiments, the Cannabinoid Product comprises about 14.3% filler by weight. In embodiments, the Cannabinoid Product comprises about 40.7% filler by weight. In embodiments, the Cannabinoid Product comprises about 43.4% filler by weight. In embodiments, the filler is mannitol. In embodiments, the filler is silicified microcrystalline cellulose.

In embodiments, the Cannabinoid Product comprises from about 20% to about 70% colloidal silicon dioxide particles by weight. In embodiments, the Cannabinoid Product comprises from about 29.1% to about 58.2% colloidal silicon dioxide particles by weight. For example, in embodiments, the Cannabinoid Product comprises about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70% colloidal silicon dioxide particles by weight, including any values, subranges, and ranges therebetween. In embodiments, the Cannabinoid Product comprises about 41.9% colloidal silicon dioxide particles by weight. In embodiments, the Cannabinoid Product comprises about 58.2% colloidal silicon dioxide particles by weight. In embodiments, the Cannabinoid Product comprises about 29.1% colloidal silicon dioxide particles by weight. In embodiments, the Cannabinoid Product comprises about 31.1% colloidal silicon dioxide particles by weight.

In embodiments, the Cannabinoid Product comprises from about 0.5% to about 10% disintegrant by weight. In embodiments, the Cannabinoid Product comprises from about 0.5% to about 5% disintegrant by weight. In embodiments, the Cannabinoid Product comprises from about 2% to about 5.5% disintegrant by weight. In embodiments, the Cannabinoid Product comprises from about 2.5% to about 7.5% disintegrant by weight. For example, in embodiments, the Cannabinoid Product comprises about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10, including any values, subranges, and ranges therebetween. In embodiments, the Cannabinoid Product comprises about 2% disintegrant by weight. In embodiments, the Cannabinoid Product comprises about 2.7% disintegrant by weight. In embodiments, the Cannabinoid Product comprises about 5.5% disintegrant by weight. In embodiments, the Cannabinoid Product comprises about 1.9% disintegrant by weight. In embodiments, the disintegrant is sodium starch glycolate or croscarmellose sodium.

In embodiments, the Cannabinoid Product comprises from about 0.5% to about 10% lubricant by weight. In embodiments, the Cannabinoid Product comprises from about 1% to about 2.7% lubricant by weight. For example, in embodiments, the Cannabinoid Product comprises about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, or about 10% lubricant by weight. In embodiments, the Cannabinoid Product comprises about 2% lubricant by weight. In embodiments, the Cannabinoid Product comprises about 2.7% lubricant by weight. In embodiments, the Cannabinoid Product comprises about 1% lubricant by weight. In embodiments, the lubricant is sodium stearyl fumarate.

In embodiments, the colloidal silicon dioxide particles described herein have an average particle size of from about 1 μm to about 100 μm. In embodiments, the colloidal silicon dioxide particles described herein have an average particle size of from about 20 μm to about 60 μm. In embodiments, the average particle size of the colloidal silicon dioxide particles is about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, or about 100 μm, including any values, subranges, and ranges therebetween.

In embodiments, the colloidal silicon dioxide particles have pores. In embodiments, the pores have a volume from 0.1 milliliters (mL) per gram (g) to about 5 mL/g. In embodiments, the pores have a volume from about 1.5 mL/g to about 1.9 mL/g. In embodiments, the pores have a volume of about 0.1 mL/g, about 0.2 mL/g, about 0.3 mL/g, about 0.4 mL/g, about 0.5 mL/g, about 0.6 mL/g, about 0.7 mL/g, about 0.8 mL/g, about 0.9 mL/g, about 1 mL/g, about 1.1 mL/g, about 1.2 mL/g, about 1.3 mL/g, about 1.4 mL/g, about 1.5 mL/g, about 1.6 mL/g, about 1.7 mL/g, about 1.8 mL/g, about 1.9 mL/g, about 2 mL/g, about 2.1 mL/g, about 2.2 mL/g, about 2.3 mL/g, about 2.4 mL/g, about 2.5 mL/g, about 2.6 mL/g, about 2.7 mL/g, about 2.8 mL/g, about 2.9 mL/g, about 3 mL/g, about 3.1 mL/g, about 3.2 mL/g, about 3.3 mL/g, about 3.4 mL/g, about 3.5 mL/g, about 3.6 mL/g, about 3.7 mL/g, about 3.8 mL/g, about 3.9 mL/g, about 4 mL/g, about 4.1 mL/g, about 4.2 mL/g, about 4.3 mL/g, about 4.4 mL/g, about 4.5 mL/g, about 4.6 mL/g, about 4.7 mL/g, about 4.8 mL/g, about 4.9 mL/g, or about 5 mL/g, including any values, subranges, and ranges therebetween.

In embodiments, the Cannabinoid Product for a tablet is selected from any formulation of Table A.

TABLE A
Exemplary Cannabinoid Products for Tablets

| | | | K (% | L (% | M (% | M[H] (% |
|---|---|---|---|---|---|---|
| | I (% w/w) | J (% w/w) | w/w) | w/w) | w/w) | w/w) |
| Cannabinoid oil | 41.9 | 22.1 | 22.1 | 22.1 | 22.1 | 19.4 |
| Colloidal silicon | 41.9 | 58.2 | 58.2 | 29.1 | 29.1 | 31.1 |
| dioxide particles | | | | | | |
| Silicified | | | | | | 46.6 |
| Microcrystalline | | | | | | |
| cellulose (filler) | | | | | | |
| Mannitol (filler) | 12.3 | 14.3 | 14.3 | 40.7 | 43.4 | |
| Sodium starch | 2.0 | 2.7 | 2.7 | 5.5 | 2.7 | |
| glycolate | | | | | | |
| (disintegrant) | | | | | | |
| Croscarmellose | | | | | | 1.9 |
| sodium | | | | | | |
| (disintegrant) | | | | | | |
| Sodium stearyl | 2.0 | 2.7 | 2.7 | 2.7 | 2.7 | 1.0 |
| fumarate | | | | | | |
| (lubricant) | | | | | | |

### III-I. Exemplary Cannabinoid Products for Capsules

In embodiments, provided herein are Cannabinoid Products for capsules. In embodiments, a Cannabinoid Product for a capsule comprises: (a) a cannabinoid oil; (b) colloidal silicon dioxide particles; and (c) a lubricant, optionally wherein the lubricant is sodium stearyl fumarate.

In embodiments, the Cannabinoid Product comprises from about 0.1% to about 70% cannabinoid oil by weight. In embodiments, the Cannabinoid Product comprises from about 0.1% to about 50% cannabinoid oil by weight. In embodiments, the Cannabinoid Product comprises from about 1% to about 50% cannabinoid oil by weight. In embodiments, the Cannabinoid Product comprises from about 0.5% to about 20% cannabinoid oil by weight. In embodiments, the Cannabinoid Product comprises from about 1% to about 10% cannabinoid oil by weight. For example, the Cannabinoid Product may comprise about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70% cannabinoid oil by weight. In embodiments, the Cannabinoid Product comprises about 2.2% cannabinoid oil by weight. In embodiments, the Cannabinoid Product comprises about 8.1% cannabinoid oil by weight.

In embodiments, the Cannabinoid Product comprises from about 25% to about 75% colloidal silicon dioxide particles by weight. In embodiments, the Cannabinoid Product comprises from about 25% to about 65% colloidal silicon dioxide particles by weight. In embodiments, the Cannabinoid Product comprises from about 25% to about 50% colloidal silicon dioxide particles by weight. In embodiments, the Cannabinoid Product comprises from about 25% to about 45% colloidal silicon dioxide particles by weight. In embodiments, the Cannabinoid Product comprises from about 25% to about 35% colloidal silicon dioxide particles by weight. For example, in embodiments, the Cannabinoid Product comprises about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, or about 75% colloidal silicon dioxide particles by weight, including any values, subranges, and ranges therebetween. In embodiments, the Cannabinoid Product comprises about 32.4% colloidal silicon dioxide particles by weight. In embodiments, the Cannabinoid Product comprises about 39% colloidal silicon dioxide particles by weight.

In embodiments, the Cannabinoid Product comprises from about 0.1% to about 1% lubricant by weight. For example, the Cannabinoid Product may comprise about 0.1%, about 0.2%, about 0.3%, about 0.4%, about 0.5%, about 0.6%, about 0.7%, about 0.8%, about 0.9%, or about 1% lubricant by weight, including any values, subranges, and ranges therebetween. In embodiments, the Cannabinoid Product comprises about 0.4% lubricant by weight. In embodiments, the lubricant is sodium stearyl fumarate.

In embodiments, the Cannabinoid Product comprises a cannabinoid isolate. In embodiments, the Cannabinoid Product comprises about 1, about 2, about 3, about 4, about 5, about 6, about 7, about 8, about 9, or about 10 cannabinoid isolates. In embodiments, the Cannabinoid Product comprises from about 1% to about 80% cannabinoid isolate by weight. In embodiments, the Cannabinoid Product comprises from about 10% to about 80% cannabinoid isolate by weight. In embodiments, the Cannabinoid Product comprises from about 10% to about 60% cannabinoid isolate by weight. In embodiments, the Cannabinoid Product comprises from about 20% to about 60% cannabinoid isolate by weight. In embodiments, the Cannabinoid Product comprises from about 20% to about 40% cannabinoid isolate by weight. In embodiments, the Cannabinoid Product comprises from about 40% to about 60% cannabinoid isolate by weight. For example, in embodiments, the Cannabinoid Product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% cannabinoid isolate by weight, including any values, subranges, and ranges therebetween. In embodiments, the Cannabinoid Product comprises cannabidiol (CBD) isolate, cannabigerol (CBG) isolate, or a combination thereof. In embodiments, the Cannabinoid Product comprises about 40.4% CBD isolate and about 11.7% CBG isolate by weight. In embodiments, the Cannabinoid Product comprises about 21.7% CBD isolate and about 8.5% CBG isolate by weight.

In embodiments, the Cannabinoid Product for a capsule comprises a filler. The filler may be any filler described herein. In embodiments, the filler is silicified microcrystalline cellulose. In embodiments, the Cannabinoid Product comprises from about 20% to about 45% filler by weight. For example, in embodiments, the Cannabinoid Product comprises about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45% filler by weight, including any values, subranges, and ranges therebetween. In embodiments, the Cannabinoid Product comprises about 25% filler by weight.

In embodiments, the Cannabinoid Product comprises an about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20 ratio of cannabinoid oil to colloidal silicon dioxide particles by weight. In embodiments, the Cannabinoid Product comprises more colloidal silicon dioxide particles than cannabinoid oil by weight.

In embodiments, the Cannabinoid Product comprises about 8.1% cannabinoid oil by weight; about 39% colloidal silicon dioxide particles by weight; and about 0.4% sodium stearyl fumarate by weight. In embodiments, the Cannabinoid Product comprises about 2.2% cannabinoid oil by weight; about 32.4% colloidal silicon dioxide particles by weight; and about 0.4% sodium stearyl fumarate by weight.

In embodiments, the cannabinoid oil is a cannabinoid distillate. In embodiments, the cannabinoid distillate comprises delta-8-tetrahydrocannabinol or is a “D8 distillate.” In embodiments, the cannabinoid distillate is flowform distillate. A flowform distillate is formulated to prevent crystallization of cannabinoids. In embodiments, a flowform distillate comprises CBD, CBG, CBN, or a combination thereof. In embodiments, the flowform distillate comprises from about 45% to about 60% CBD by weight. In embodiments, the flowform distillate comprises greater than 2.5% CBG by weight. In embodiments, the flowform distillate comprises from about 45% to about 60% CBD by weight and greater than 2.5% CBG by weight. In embodiments, the flowform distillate comprises about 52.5% CBD and about 14.6% CBG by weight. In embodiments, the flowform distillate comprises about 7.4% cannabielsoin (CBE) by weight. In embodiments, the flowform distillate comprises about 2.6% CBDV by weight. In embodiments, the flowform distillate comprises about 0.1% THC by weight.

In embodiments, the colloidal silicon dioxide particles described herein have an average particle size of from about 1 μm to about 100 μm. In embodiments, the colloidal silicon dioxide particles described herein have an average particle size of from about 20 μm to about 60 μm. In embodiments, the average particle size of the colloidal silicon dioxide particles is about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, or about 100 μm, including any values, subranges, and ranges therebetween.

In embodiments, the colloidal silicon dioxide particles have pores. In embodiments, the pores have a volume from 0.1 milliliters (mL) per gram (g) to about 5 mL/g. In embodiments, the pores have a volume from about 1.5 mL/g to about 1.9 mL/g. In embodiments, the pores have a volume of about 0.1 mL/g, about 0.2 mL/g, about 0.3 mL/g, about 0.4 mL/g, about 0.5 mL/g, about 0.6 mL/g, about 0.7 mL/g, about 0.8 mL/g, about 0.9 mL/g, about 1 mL/g, about 1.1 mL/g, about 1.2 mL/g, about 1.3 mL/g, about 1.4 mL/g, about 1.5 mL/g, about 1.6 mL/g, about 1.7 mL/g, about 1.8 mL/g, about 1.9 mL/g, about 2 mL/g, about 2.1 mL/g, about 2.2 mL/g, about 2.3 mL/g, about 2.4 mL/g, about 2.5 mL/g, about 2.6 mL/g, about 2.7 mL/g, about 2.8 mL/g, about 2.9 mL/g, about 3 mL/g, about 3.1 mL/g, about 3.2 mL/g, about 3.3 mL/g, about 3.4 mL/g, about 3.5 mL/g, about 3.6 mL/g, about 3.7 mL/g, about 3.8 mL/g, about 3.9 mL/g, about 4 mL/g, about 4.1 mL/g, about 4.2 mL/g, about 4.3 mL/g, about 4.4 mL/g, about 4.5 mL/g, about 4.6 mL/g, about 4.7 mL/g, about 4.8 mL/g, about 4.9 mL/g, or about 5 mL/g, including any values, subranges, and ranges therebetween.

In embodiments, the Cannabinoid Product for a capsule is selected from any one of the formulations of Table B.

TABLE B

| | G[C] (% | F[C] (% | E[C] (% | D[C] (% |
|---|---|---|---|---|
| | w/w) | w/w) | w/w) | w/w) |
| Cannabidiol (CBD) | 40.4 | 40.4 | 40.4 | 21.7 |
| Isolate | | | | |
| Cannabigerol (CBG) | 11.7 | 11.7 | 11.7 | 8.5 |
| Isolate | | | | |
| Cannabinoid oil | 8.1 | 8.1 | 8.1 | 2.2 |
| Colloidal silicon dioxide | 39 | 39 | 39 | 32.4 |
| Silicified Microcrystalline | | | | 34.8 |
| cellulose (filler) | | | | |
| Sodium stearyl fumarate | 0.4 | 0.4 | 0.4 | 0.4 |
| (lubricant) | | | | |
| Myrcene (flavoring) | 0.2 | 0.2 | 0.2 | |
| Linalool (flavoring) | 0.2 | 0.2 | 0.2 | |

### IV. Methods of Making Cannabinoid Products

Provided herein are methods of making the Cannabinoid Products described herein. In each of the methods for making Cannabinoid Products described herein, the initial step comprises mixing the cannabinoid oil and colloidal silicon dioxide particles. In embodiments, the cannabinoid oil is added to the colloidal silicon dioxide particles. In embodiments, the colloidal silicon dioxide particles are added to the cannabinoid distillate.

In embodiments, the initial step comprises mixing cannabinoid oil and colloidal silicon dioxide particles with cannabinoid isolate and/or terpenes.

In embodiments, a mixing step of the methods described herein comprises high-shear mixing using a high shear mixer. The term “high shear mixer” refers to an apparatus that disperses a first ingredient (e.g., a cannabinoid oil) into a second ingredient (e.g., colloidal silicon dioxide particles) using mechanical agitation. In embodiments, the high shear mixer comprises a rotor or an impeller with a stationary component called a stator. Non-limiting examples of high-shear mixers include batch high shear mixers, inline high shear mixers, ultra high shear inline mixers, grinding mills, and grinders. In embodiments, the high-shear mixer is a coffee grinder.

In embodiments, the high shear mixer operates at a speed of from 500 revolutions per minutes (rpm) to about 5000 rpm. In embodiments, the high shear mixer operates at a speed of from 1000 rpm to about 2000 rpm. For example, in embodiments, the speed of the high shear mixer is about 500 rpm, about 600 rpm, about 700 rpm, about 800 rpm, about 900 rpm, about 1000 rpm, about 1100 rpm, about 1200 rpm, about 1300 rpm, about 1400 rpm, about 1500 rpm, about 1600 rpm, about 1700 rpm, about 1800 rpm, about 1900 rpm, about 2000 rpm, about 2100 rpm, about 2200 rpm, about 2300 rpm, about 2400 rpm, about 2500 rpm, about 2600 rpm, about 2700 rpm, about 2800 rpm, about 2900 rpm, about 3000 rpm, about 3100 rpm, about 3200 rpm, about 3300 rpm, about 3400 rpm, about 3500 rpm, about 3600 rpm, about 3700 rpm, about 3800 rpm, about 3900 rpm, about 4000 rpm, about 4100 rpm, about 4200 rpm, about 4300 rpm, about 4400 rpm, about 4500 rpm, about 4600 rpm, about 4700 rpm, about 4800 rpm, about 4900 rpm, or about 5000 rpm, including all values, subranges, and ranges therebetween.

In embodiments, high-shear mixing comprises placing a first ingredient and second ingredient in a high-shear mixer and pulsing the mixture. In embodiments, the mixture is exposed to a pulse that lasts for about 1 second, about 2 seconds, about 3 seconds, about 4 seconds, about 5 seconds, about 6 seconds, about 7 seconds, about 8 seconds, about 9 seconds, or about 10 seconds. In embodiments, the first ingredient and second ingredient are mixed in the high-shear mixer for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 pulses, including all values, subranges, and ranges therebetween.

In embodiments, the methods described herein comprise high-shear mixing for from about 1 second (sec) to about 1 minute (min). In embodiments, the methods described herein comprise high-shear mixing for from about 1 sec to about 5 min. In embodiments, the methods described herein comprise high-shear mixing for from about 1 sec to about 10 min. In embodiments, the methods described herein comprise high-shear mixing for from about 1 secto about 15 min. In embodiments, the methods described herein comprise high-shear mixing for from about 1 sec to about 20 min. In embodiments, the methods described herein comprise high-shear mixing for from about 1 sec to about 25 min. In embodiments, the methods described herein comprise high-shear mixing for from about 1 sec to about 30 min. In embodiments, the methods described herein comprise high-shear mixing for less than a minute, up to 20 min, at least 1 min, at least 5 min, at least 10 min, at least 15 min, or at least 20 min. For example, in embodiments, the methods described herein comprise high-shear mixing for about 1 sec, about 2 sec, about 3 sec, about 4 sec, about 5 sec, about 6 sec, about 7 sec, about 8 sec, about 9 sec, about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, or about 30 min, including all values, subranges, and ranges therebetween.

In embodiments, the methods of making the Cannabinoid Products described herein comprise additional forms of mixing. Non-limiting examples of mixing include a stand mixing, bag-mixing, and v-blending. In embodiments, any combination of the ingredients within the Cannabinoid Products may be mixed using a mixing element selected from the group consisting of: spatulas, paddles, mechanical mixers, mechanical stirrers, stir bars, media dispensers, solid particles that aid in mixing the compounds (e.g., mixing balls), overhead mixers, static mixers, a v-blender, a bag-mixer, a stand-mixer, or a mortar and a pestle.

In embodiments, a composition comprising colloidal silicon dioxide particles and cannabinoid oil is mixed with one or more of a lubricant, a filler, a disintegrant, or a flavoring. In embodiments, mixing occurs from about 1 sec to 24 hours (h). For example, in embodiments, mixing occurs for about 1 sec, about 2 sec, about 3 sec, about 4 sec, about 5 sec, about 6 sec, about 7 sec, about 8 sec, about 9 sec, about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 45 min, about 1 h, about 1.5 h, about 2 h, about 2.5 h, about 3 h, about 3.5 h, about 4 h, about 4.5 h, about 5 h, about 5.5 h, about 6 h, about 6.5 h, about 7 h, about 7.5 h, about 8 h, about 8.5 h, about 9 h, about 9.5 h, about 10 h, about 10.5 h, about 11 h, about 11.5 h, about 12 h, about 12.5 h, about 13 h, about 13.5 h, about 14 h, about 14.5 h, about 15 h, about 15.5 h, about 16 h, about 16.5 h, about 17 h, about 17.5 h, about 18 h, about 18.5 h, about 19 h, about 19.5 h, about 20 h, about 20.5 h, about 21 h, about 21.5 h, about 22 h, about 22.5 h, about 23 h, about 23.5 h, or about 24 h, including all subranges, ranges, and values therebetween.

In embodiments, when a lubricant is added to the Cannabinoid Product, the lubricant is the final ingredient added to the Cannabinoid Product. In embodiments, mixing of the lubricant with other ingredients of the Cannabinoid Product occurs from about 10 sec to about 5 min. For example, in embodiments, mixing occurs for about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, about 5 min, including all subranges, ranges, and values therebetween. In embodiments, mixing of the lubricant with other ingredients of the Cannabinoid Product occurs from about 1 min to about 2 min.

In embodiments, the methods of creating a Cannabinoid Product comprise heating the cannabinoid oil. In embodiments, the methods of creating a Cannabinoid Product comprise heating a compositoi comprising the cannabinoid oil and colloidal silicon dioxide particles. In embodiments, the cannabinoid oil is heated before the extract oil is mixed with the colloidal silicon dioxide particles. In embodiments, the cannabinoid oil is heated to at least 30° C., at least 40° C., at least 50° C., at least 60° C., or at least 70° C. In embodiments, the cannabinoid oil is heated to about 60° C. In embodiments, the cannabinoid oil is heated to a temperature ranging from about 30° C. to about 70° C., including all values, ranges, and subranges therebetween. In embodiments, the cannabinoid oil is heated to a temperature ranging from about 30° C. to about 65° C., including all values, ranges, and subranges therebetween. In embodiments, the cannabinoid oil is heated to a temperature ranging from about 30° C. to about 60° C., including all values, ranges, and subranges therebetween. In embodiments, the cannabinoid oil is heated to about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C. In embodiments, the cannabinoid oil is not heated to a temperature higher than about 60° C. In embodiments, the cannabinoid oil is not heated to a temperature higher than about 65° C. In embodiments, the cannabinoid oil is not heated to a temperature higher than about 70° C. Without being bound by theory, a cannabinoid oil is not heated to a temperature above 70° C. because temperatures above 70° C. degrade cannabinoids.

In embodiments, the methods of making Cannabinoid Products comprise diluting the cannabinoid oil in a solvent before the oil is mixed with the colloidal silicon dioxide particles. In embodiments, the solvent is selected from the group consisting of ethanol, methanol, propanol, olive oil, coconut oil, canola oil, flaxseed oil, avocado oil, sesame oil, canola oil, palm oil, safflower oil, soybean oil, corn oil, peanut oil, walnut oil, flaxseed oil, sunflower oil, palm oil, palm kernel oil, caproic acid, caprylic acid, hempseed oil, walnut oil, or medium-chain triglyceride (MCT) oil. In embodiments, the solvent is ethanol. Without being bound by theory, initial dilution of cannabinoid oil in a solvent helps with absorption of the cannabinoid oil into the colloidal silicon dioxide particle pores.

### IV-A. Exemplary Methods of Making Cannabinoid Products for Tablets

In embodiments, provided herein is a method of making a Cannabinoid Product comprising: (a) mixing the cannabinoid oil and colloidal silicon dioxide particles; (b) adding the filler and disintegrant to the mixture of distillate and colloidal silicon dioxide particles; and (c) adding the lubricant. Optionally, the filler is silicified microcrystalline cellulose or mannitol. Optionally, the disintegrant is sodium starch glycolate or croscarmellose sodium. Optionally, the lubricant is sodium stearyl fumarate.

In embodiments, step (a) is performed before step (b). In embodiments, step (b) is performed before step (c). In embodiments, step (a) is performed before step (b), which is performed before step (c).

In embodiments, the filler and disintegrant are added to a composition containing cannabinoid oil and colloidal silicon dioxide particles, and the resultant composition is mixed. In embodiments, mixing occurs for about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, or about 20 min. In embodiments, mixing occurs for about 3 minutes.

In embodiments, the method of making the Cannabinoid Product comprises mixing the cannabinoid oil with ethanol. In embodiments, the cannabinoid oil and ethanol mixture are heated (e.g., in an oven). In embodiments, the method of making the Cannabinoid Product comprises heating a composition comprising the cannabinoid and colloidal silicon dioxide particles. In embodiments, the oven or other heating device is set to a temperature of at least about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C. In embodiments, the oven temperature is about 60° C. In embodiments, the oven temperature is about 70° C. In embodiments, the heated cannabinoid oil is added to the colloidal silicon dioxide particles. In embodiments, the cannabinoid oil is added to the colloidal silicon dioxide particles in a drop-wise fashion. In embodiments, a filler and disintegrant are added the to the composition containing cannabinoid oil and colloidal silicon dioxide particles. In embodiments, the lubricant is added to the composition containing cannabinoid oil, colloidal silicon dioxide particles, filler, and disintegrant. In embodiments, the lubricant is and composition containing cannabinoid oil, colloidal silicon dioxide particles, filler, and disintegrant are mixed for less than 5 minutes, less than 4 minutes, less than 3 minutes, less than 2 minutes, or less than 1 minute.

In embodiments, the method of making the Cannabinoid Product comprises heating the cannabinoid oil (e.g., in an oven). In embodiments, the method of making the Cannabinoid Product comprises heating a composition comprising the cannabinoid and colloidal silicon dioxide particles. In embodiments, the oven or other heating device is set to a temperature of at least about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C. In embodiments, the oven temperature is about 60° C. In embodiments, colloidal silicon dioxide particles and cannabinoid oil are mixed in a high-shear mixer (e.g., a grinder). In embodiments, the composition is pulsed in the high-shear mixer for about 15 to about 30 pulses, wherein each pulse lasts one second. Optionally, the composition is grinded continuously for about 20 minutes until it is homogenous. In embodiments, a filler and disintegrant are added the to the composition containing cannabinoid oil and colloidal silicon dioxide particles. In embodiments, the filler and disintegrant are mixed with the composition containing cannabinoid oil and colloidal silicon dioxide particles for about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, or about 20 min. In embodiments, the lubricant is and composition containing cannabinoid oil, colloidal silicon dioxide particles, filler, and disintegrant are mixed for less than 5 minutes, less than 4 minutes, less than 3 minutes, less than 2 minutes, or less than 1 minute.

### IV-B. Exemplary Methods of Making Cannabinoid Products for Capsules

In embodiments, provided herein is a method of making a Cannabinoid Product comprising: (a) mixing the cannabinoid oil and silicon dioxide particles; and (b) adding the lubricant. In embodiments, the method further comprises (c) adding the filler. In embodiments, step (a) is performed before step (b). In embodiments, step (a) is performed before step (c) and step (c) is performed before step (b).

In embodiments, the cannabinoid oil and silicon dioxide particles are combined by mixing with a mortar and pestle. In embodiments, the composition containing the colloidal silicon dioxide particles and cannabinoid oil are combined with cannabinoid isolates (e.g., CBG and/or CBD isolate) and/or filler and mixed. In embodiments, mixing occurs in a stand mixer. In embodiments, mixing occurs for about 1 min, about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, or about 20 min. In embodiments, lubricant is added. In embodiments, mixing of the lubricant with the composition containing colloidal silicon dioxide particles, cannabinoid oil, cannabinoid isolates, and/or filler is performed for from 1 second to about 5 minutes. For example, in embodiments, mixing occurs for about 1 sec, about 5 sec, about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, or about 5 min.

In embodiments, cannabinoid oil, silicon dioxide particles, and optionally one or more terpenes (e.g., linalool and/or myrcene) are mixed in a grinder until the composition is uniform. In embodiments, the composition containing cannabinoid oil, silicon dioxide particles, and optionally one or more terpenes is transferred to a mixing bowl, and optionally cannabinoid isolate (e.g., CBD and/or CBG isolate) is added to the bowl, and the mixture is mixed for 1-20 minutes in a stand mixer. In embodiments, the mixture is mixed for about 5 min. In embodiments, the mixture is mixed for about 10 min. In embodiments, lubricant is added to the bowl. In embodiments, the lubricant is mixed with the cannabinoid oil, silicon dioxide particles, optionally one or more terpenes, and cannabinoid isolate for about 1 sec, about 5 sec, about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, or about 5 min. In embodiments, the lubricant is mixed with the cannabinoid oil, silicon dioxide particles, optionally one or more terpenes, and optionally cannabinoid isolate for about 2 min. In embodiments, the lubricant is mixed with the cannabinoid oil, silicon dioxide particles, optionally one or more terpenes, and optionally cannabinoid isolate for about 1 min.

### V. Capsules and Tablets and Methods of Making the Same

Provided herein are tablets and capsules comprising the Cannabinoid Products of Section III.

### V-A. Parameters of Tablets

In embodiments, the mass of the tablets ranges from about 100 mg to about 800 mg. In embodiments, the mass of the tablets ranges from about 100 mg to about 200 mg. In embodiments, the mass of the tablets ranges from about 500 mg to about 600 mg. In embodiments, the mass of the tablets ranges from about 700 mg to about 800 mg. In embodiments, the tablets described herein are about 103 mg.

In embodiments, the tablet diameter ranges from about 4 mm to about 25 mm. In embodiments, the tablet diameter is about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, about 10.1 mm, about 10.2 mm, about 10.3 mm, about 10.4 mm, about 10.5 mm, about 10.6 mm, about 10.7 mm, about 10.8 mm, about 10.9 mm, about 11 mm, about 11.1 mm, about 11.2 mm, about 11.3 mm, about 11.4 mm, about 11.5 mm, about 11.6 mm, about 11.7 mm, about 11.8 mm, about 11.9 mm, about 12 mm, about 12.1 mm, about 12.2 mm, about 12.3 mm, about 12.4 mm, about 12.5 mm, about 12.6 mm, about 12.7 mm, about 12.8 mm, about 12.9 mm, about 13 mm, about 13.1 mm, about 13.2 mm, about 13.3 mm, about 13.4 mm, about 13.5 mm, about 13.6 mm, about 13.7 mm, about 13.8 mm, about 13.9 mm, about 14 mm, about 14.1 mm, about 14.2 mm, about 14.3 mm, about 14.4 mm, about 14.5 mm, about 14.6 mm, about 14.7 mm, about 14.8 mm, about 14.9 mm, about 15 mm, about 15.1 mm, about 15.2 mm, about 15.3 mm, about 15.4 mm, about 15.5 mm, about 15.6 mm, about 15.7 mm, about 15.8 mm, about 15.9 mm, about 16 mm, about 16.1 mm, about 16.2 mm, about 16.3 mm, about 16.4 mm, about 16.5 mm, about 16.6 mm, about 16.7 mm, about 16.8 mm, about 16.9 mm, about 17 mm, about 17.1 mm, about 17.2 mm, about 17.3 mm, about 17.4 mm, about 17.5 mm, about 17.6 mm, about 17.7 mm, about 17.8 mm, about 17.9 mm, about 18 mm, about 18.1 mm, about 18.2 mm, about 18.3 mm, about 18.4 mm, about 18.5 mm, about 18.6 mm, about 18.7 mm, about 18.8 mm, about 18.9 mm, about 19 mm, about 19.1 mm, about 19.2 mm, about 19.3 mm, about 19.4 mm, about 19.5 mm, about 19.6 mm, about 19.7 mm, about 19.8 mm, about 19.9 mm, about 20 mm, about 20.1 mm, about 20.2 mm, about 20.3 mm, about 20.4 mm, about 20.5 mm, about 20.6 mm, about 20.7 mm, about 20.8 mm, about 20.9 mm, about 21 mm, about 21.1 mm, about 21.2 mm, about 21.3 mm, about 21.4 mm, about 21.5 mm, about 21.6 mm, about 21.7 mm, about 21.8 mm, about 21.9 mm, about 22 mm, about 22.1 mm, about 22.2 mm, about 22.3 mm, about 22.4 mm, about 22.5 mm, about 22.6 mm, about 22.7 mm, about 22.8 mm, about 22.9 mm, about 23 mm, about 23.1 mm, about 23.2 mm, about 23.3 mm, about 23.4 mm, about 23.5 mm, about 23.6 mm, about 23.7 mm, about 23.8 mm, about 23.9 mm, about 24 mm, about 24.1 mm, about 24.2 mm, about 24.3 mm, about 24.4 mm, about 24.5 mm, about 24.6 mm, about 24.7 mm, about 24.8 mm, about 24.9 mm, or about 25 mm, including all subranges, ranges, and values therebetween.

In embodiments, the tablet shape is selected from flat faced, shallow convey, normal convex, deep convex, ball or pill, flat bevelled edge, double radius, bevel and convex, dimple, ring, rim, capsule, oval, ellipse, square, triangle, pentagon, hexagon, heptagon, octagon, diamond, pillow, barrel, rectangle, almond, arrow head, bullet, half moon, shield, heart, star, or round. In embodiments, the tablet shape is round. In embodiments, the tablet shape is round with a flat face bevel edge. In embodiments, the tablet shape is round and standard convex.

In embodiments, the tablets have a hardness that ranges from 50 Newtons (N) to about 150 N. In embodiments, the tablets have a hardness that ranges from 50 N to about 100 N. In embodiments, the tablets have a hardness that ranges from 100 N to about 150 N. In embodiments, the tablets have a hardness that ranges from 60 N to about 70 N. For example, in embodiments, the tablets have a hardness of about 50 N, about 51 N, about 52 N, about 53 N, about 54 N, about 55 N, about 56 N, about 57 N, about 58 N, about 59 N, about 60 N, about 61 N, about 62 N, about 63 N, about 64 N, about 65 N, about 66 N, about 67 N, about 68 N, about 69 N, about 70 N, about 71 N, about 72 N, about 73 N, about 74 N, about 75 N, about 76 N, about 77 N, about 78 N, about 79 N, about 80 N, about 81 N, about 82 N, about 83 N, about 84 N, about 85 N, about 86 N, about 87 N, about 88 N, about 89 N, about 90 N, about 91 N, about 92 N, about 93 N, about 94 N, about 95 N, about 96 N, about 97 N, about 98 N, about 99 N, about 100 N, about 101 N, about 102 N, about 103 N, about 104 N, about 105 N, about 106 N, about 107 N, about 108 N, about 109 N, about 110 N, about 111 N, about 112 N, about 113 N, about 114 N, about 115 N, about 116 N, about 117 N, about 118 N, about 119 N, about 120 N, about 121 N, about 122 N, about 123 N, about 124 N, about 125 N, about 126 N, about 127 N, about 128 N, about 129 N, about 130 N, about 131 N, about 132 N, about 133 N, about 134 N, about 135 N, about 136 N, about 137 N, about 138 N, about 139 N, about 140 N, about 141 N, about 142 N, about 143 N, about 144 N, about 145 N, about 146 N, about 147 N, about 148 N, about 149 N, or about 150 N, including all values, subranges, and ranges therebetween. In embodiments, the tablets have a hardness of about 50 N, at least about 51 N, at least about 52 N, at least about 53 N, at least about 54 N, at least about 55 N, at least about 56 N, at least about 57 N, at least about 58 N, at least about 59 N, at least about 60 N, at least about 61 N, at least about 62 N, at least about 63 N, at least about 64 N, at least about 65 N, at least about 66 N, at least about 67 N, at least about 68 N, at least about 69 N, at least about 70 N, at least about 71 N, at least about 72 N, at least about 73 N, at least about 74 N, at least about 75 N, at least about 76 N, at least about 77 N, at least about 78 N, at least about 79 N, at least about 80 N, at least about 81 N, at least about 82 N, at least about 83 N, at least about 84 N, at least about 85 N, at least about 86 N, at least about 87 N, at least about 88 N, at least about 89 N, at least about 90 N, at least about 91 N, at least about 92 N, at least about 93 N, at least about 94 N, at least about 95 N, at least about 96 N, at least about 97 N, at least about 98 N, at least about 99 N, at least about 100 N, at least about 101 N, at least about 102 N, at least about 103 N, at least about 104 N, at least about 105 N, at least about 106 N, at least about 107 N, at least about 108 N, at least about 109 N, at least about 110 N, at least about 111 N, at least about 112 N, at least about 113 N, at least about 114 N, at least about 115 N, at least about 116 N, at least about 117 N, at least about 118 N, at least about 119 N, at least about 120 N, at least about 121 N, at least about 122 N, at least about 123 N, at least about 124 N, at least about 125 N, at least about 126 N, at least about 127 N, at least about 128 N, at least about 129 N, at least about 130 N, at least about 131 N, at least about 132 N, at least about 133 N, at least about 134 N, at least about 135 N, at least about 136 N, at least about 137 N, at least about 138 N, at least about 139 N, at least about 140 N, at least about 141 N, at least about 142 N, at least about 143 N, at least about 144 N, at least about 145 N, at least about 146 N, at least about 147 N, at least about 148 N, at least about 149 N, or at least about 150 N. In embodiments, large tablets (tablets with a mass of 500 mg or larger) have a hardness from about 50 N to about 150 N. In embodiments, small tablets (tablets with a mass of from about 100 mg to about 300 mg) have a hardness from about 50 N to about 100 N.

In embodiments, the tablets have a compression force ranging from about 1 kilonewton (kN) to about 30 kN. In embodiments, the tablets have a compression force ranging from about 5 kN to about 20 kN. In embodiments, the tablets have a compression force ranging from about 10 kN to about 16 kN. For example, the compression force of a tablet is about 1 kN, about 2 kN, about 3 kN, about 4 kN, about 5 kN, about 6 kN, about 7 kN, about 8 kN, about 9 kN, about 10 kN, about 11 kN, about 12 kN, about 13 kN, about 14 kN, about 15 kN, about 16 kN, about 17 kN, about 18 kN, about 19 kN, about 20 kN, about 21 kN, about 22 kN, about 23 kN, about 24 kN, about 25 kN, about 26 kN, about 27 kN, about 28 kN, about 29 kN, or about 30 kN, including all values, subranges, and ranges therebetween. In embodiments, the compression force of a tablet is at least about 1 kN, at least about 2 kN, at least about 3 kN, at least about 4 kN, at least about 5 kN, at least about 6 kN, at least about 7 kN, at least about 8 kN, at least about 9 kN, at least about 10 kN, at least about 11 kN, at least about 12 kN, at least about 13 kN, at least about 14 kN, at least about 15 kN, at least about 16 kN, at least about 17 kN, at least about 18 kN, at least about 19 kN, at least about 20 kN, at least about 21 kN, at least about 22 kN, at least about 23 kN, at least about 24 kN, at least about 25 kN, at least about 26 kN, at least about 27 kN, at least about 28 kN, at least about 29 kN, or at least about 30 kN.

In embodiments, the tablet substantially disintegrates in from about 10 sec to about 10 min. For example, in embodiments, the tablet substantially disintegrates in within about 10 sec, about 15 sec, about 20 sec, about 25 sec, about 30 sec, about 35 sec, about 40 sec, about 45 see, about 50 sec, about 55 sec, about 60 sec, about 70 sec, about 80 sec, about 90 sec, about 2 min, about 2.5 min, about 3 min, about 3.5 min, about 4 min, about 4.5 min, about 5 min, about 5.5 min, about 6 min, about 6.5 min, about 7 min, about 7.5 min, about 8 min, about 8.5 min, about 9 min, about 9.5 min, or about 10 min including all values, subranges, and ranges therebetween. In embodiments, the tablet substantially disintegrates in 2% sodium lauryl sulfate in less than 5 minutes, less than 4 min, less than 3 min, less than 2 min, less than 1 min, or less than 30 sec.

In embodiments, the tablet has a Percentage Dissolved at 60 minutes of at least about 60%, at least about 65%, at least about 70%, at least about 75%, at least about 80%, at least about 85%, at least about 90%, at least about 95%, or more in a solvent. In embodiments, the solvent is 1% w/v Tween 80 (polysorbate 80) solution, fed state simulated intestinal fluid (“FeSSIF,” contains about 15 mM taurocholate, 3.75 mM phospholipids, 319 mM sodium ions, 203 mM chloride, and 144 mM acetic acid), fasted state simulated intestinal fluid (“FaSSIF,” contains 3 mM taurocholate, 0.75 mM phospholipids, 148 mM sodium ions, 106 mM chloride, and 29 mM phosphate ions).

In embodiments, a tablet containing a Cannabinoid Product containing cannabinoid oil and colloidal silicon dioxide particles described herein has a Percentage Dissolved at 60 minutes, which is improved relative to a tablet containing a Cannabinoid Product that contains cannabinoid oil, but lacks colloidal silicon dioxide particles. In embodiments, the Percentage Dissolved at 60 minutes is improved by about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9, about 10, or more fold. In embodiments, the Percentage Dissolved at 60 minutes is improved by at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, at least about 2.5, at least about 2.6, at least about 2.7, at least about 2.8, at least about 2.9, at least about 3, at least about 3.1, at least about 3.2, at least about 3.3, at least about 3.4, at least about 3.5, at least about 3.6, at least about 3.7, at least about 3.8, at least about 3.9, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.1, at least about 9.2, at least about 9.3, at least about 9.4, at least about 9.5, at least about 9.6, at least about 9.7, at least about 9.8, at least about 9.9, at least about 10, or more fold.

In embodiments, the tablet is an orodispersable tablet or immediate release tablet.

### V-B. Parameters of Capsules

In embodiments, the capsules described herein encapsulate Cannabinoid Products via a capsule shell. In embodiments, the capsule shell comprises gelatin, hydroxypropylmethylcellulose, or a combination thereof.

In embodiments, the capsule has a Percentage Dissolved at 60 minutes of from about 5% to about 60% in a solvent. In embodiments, the capsule has a Percentage Dissolved at 60 minutes of from about 18.1% to about 42.3% in a solvent. In embodiments, the capsule has a Percentage Dissolved at 60 minutes of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more in a solvent. In embodiments, the solvent is 1% w/v Tween 80 (polysorbate 80) solution, fed state simulated intestinal fluid (“FeSSIF,” contains about 15 mM taurocholate, 3.75 mM phospholipids, 319 mM sodium ions, 203 mM chloride, and 144 mM acetic acid), fasted state simulated intestinal fluid (“FaSSIF,” contains 3 mM taurocholate, 0.75 mM phospholipids, 148 mM sodium ions, 106 mM chloride, and 29 mM phosphate ions).

In embodiments, a capsule containing a Cannabinoid Product containing cannabinoid oil and colloidal silicon dioxide particles described herein has a Percentage Dissolved at 60 minutes, which is improved relative to a capsule containing a Cannabinoid Product that contains cannabinoid oil, but lacks colloidal silicon dioxide particles. In embodiments, the Percentage Dissolved at 60 minutes is improved by about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9, about 10, or more fold. In embodiments, the Percentage Dissolved at 60 minutes is improved by at least about 2, at least about 2.1, at least about 2.2, at least about 2.3, at least about 2.4, at least about 2.5, at least about 2.6, at least about 2.7, at least about 2.8, at least about 2.9, at least about 3, at least about 3.1, at least about 3.2, at least about 3.3, at least about 3.4, at least about 3.5, at least about 3.6, at least about 3.7, at least about 3.8, at least about 3.9, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.1, at least about 9.2, at least about 9.3, at least about 9.4, at least about 9.5, at least about 9.6, at least about 9.7, at least about 9.8, at least about 9.9, at least about 10, or more fold. In embodiments, the Percentage Dissolved at 60 minutes is improved by at least about 4.4 fold.

### V-C. Methods of Making Tablets

In embodiments, the Cannabinoid Products described herein are compressed into tablets using a tablet press. A tablet press uses a compression force to transform a powder, e.g., a Cannabinoid Product, into a tablet. Tablet presses comprise a tooling made of dies and punches. The die is a cavity where the powder for the tablet, e.g., the Cannabinoid Product is added. The die determines the diameter, size, and thickness of a tablet. The punches (upper and lower) compress the powder into tablets of various shapes within the die. The tablet tooling may be classified as “B,” “D,” “BB,” or “DB,” according to the diameter of the punch barrel and the outer diameter of the die. (Table C).

TABLE C
Types of Tablet Press Tooling

| Type of Tablet | Diameter of the | Outer diameter of |
|---|---|---|
| Press Tooling | Punch Barrel (mm) | the die (mm) |
| B | 19 | 30.16 |
| D | 25.4 | 38.10 |
| BB | 19.0 | 24.00 |
| DB | 25.4 | 30.16 |

In embodiments, the tablet press is a “single punch tablet press machine.” Single punch tablet press machines use a single set of station tooling (e.g., die and punches) to process a powder into the desired tablet size. In embodiments, the tablet press is a rotary tablet press. A rotary press has multiple sets of dies with lower and upper punches, which are in constant rotary motion. In embodiments, the rotary press is the Globepharma Mini Press-II. The Globepharma Mini Press-II has five stations of B tooling and five stations of D tooling. The Globepharma Mini Press-II also has a gravity hopper and gravity feeder which allows Cannabinoid Product to flow into the die.

In embodiments, the diameter of the die (also referred to as “tablet diameter of tooling”) determines the tablet diameter. In embodiments, the diameter of the die ranges from 4 mm to about 25 mm. In embodiments, the diameter of the die is about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, about 10.1 mm, about 10.2 mm, about 10.3 mm, about 10.4 mm, about 10.5 mm, about 10.6 mm, about 10.7 mm, about 10.8 mm, about 10.9 mm, about 11 mm, about 11.1 mm, about 11.2 mm, about 11.3 mm, about 11.4 mm, about 11.5 mm, about 11.6 mm, about 11.7 mm, about 11.8 mm, about 11.9 mm, about 12 mm, about 12.1 mm, about 12.2 mm, about 12.3 mm, about 12.4 mm, about 12.5 mm, about 12.6 mm, about 12.7 mm, about 12.8 mm, about 12.9 mm, about 13 mm, about 13.1 mm, about 13.2 mm, about 13.3 mm, about 13.4 mm, about 13.5 mm, about 13.6 mm, about 13.7 mm, about 13.8 mm, about 13.9 mm, about 14 mm, about 14.1 mm, about 14.2 mm, about 14.3 mm, about 14.4 mm, about 14.5 mm, about 14.6 mm, about 14.7 mm, about 14.8 mm, about 14.9 mm, about 15 mm, about 15.1 mm, about 15.2 mm, about 15.3 mm, about 15.4 mm, about 15.5 mm, about 15.6 mm, about 15.7 mm, about 15.8 mm, about 15.9 mm, about 16 mm, about 16.1 mm, about 16.2 mm, about 16.3 mm, about 16.4 mm, about 16.5 mm, about 16.6 mm, about 16.7 mm, about 16.8 mm, about 16.9 mm, about 17 mm, about 17.1 mm, about 17.2 mm, about 17.3 mm, about 17.4 mm, about 17.5 mm, about 17.6 mm, about 17.7 mm, about 17.8 mm, about 17.9 mm, about 18 mm, about 18.1 mm, about 18.2 mm, about 18.3 mm, about 18.4 mm, about 18.5 mm, about 18.6 mm, about 18.7 mm, about 18.8 mm, about 18.9 mm, about 19 mm, about 19.1 mm, about 19.2 mm, about 19.3 mm, about 19.4 mm, about 19.5 mm, about 19.6 mm, about 19.7 mm, about 19.8 mm, about 19.9 mm, about 20 mm, about 20.1 mm, about 20.2 mm, about 20.3 mm, about 20.4 mm, about 20.5 mm, about 20.6 mm, about 20.7 mm, about 20.8 mm, about 20.9 mm, about 21 mm, about 21.1 mm, about 21.2 mm, about 21.3 mm, about 21.4 mm, about 21.5 mm, about 21.6 mm, about 21.7 mm, about 21.8 mm, about 21.9 mm, about 22 mm, about 22.1 mm, about 22.2 mm, about 22.3 mm, about 22.4 mm, about 22.5 mm, about 22.6 mm, about 22.7 mm, about 22.8 mm, about 22.9 mm, about 23 mm, about 23.1 mm, about 23.2 mm, about 23.3 mm, about 23.4 mm, about 23.5 mm, about 23.6 mm, about 23.7 mm, about 23.8 mm, about 23.9 mm, about 24 mm, about 24.1 mm, about 24.2 mm, about 24.3 mm, about 24.4 mm, about 24.5 mm, about 24.6 mm, about 24.7 mm, about 24.8 mm, about 24.9 mm, or about 25 mm. For example, in embodiments, the diameter of the die is about 4 mm, about 4.1 mm, about 4.2 mm, In embodiments, the diameter of the die is about 6.35 mm (about 0.25 inches). In embodiments, the diameter of the die is about 7.94 mm (about 0.3125 inches). In embodiments, the diameter of the die is about 7 mm. In embodiments, the diameter of the die is about 13 mm.

### V-D. Methods of Making Capsules

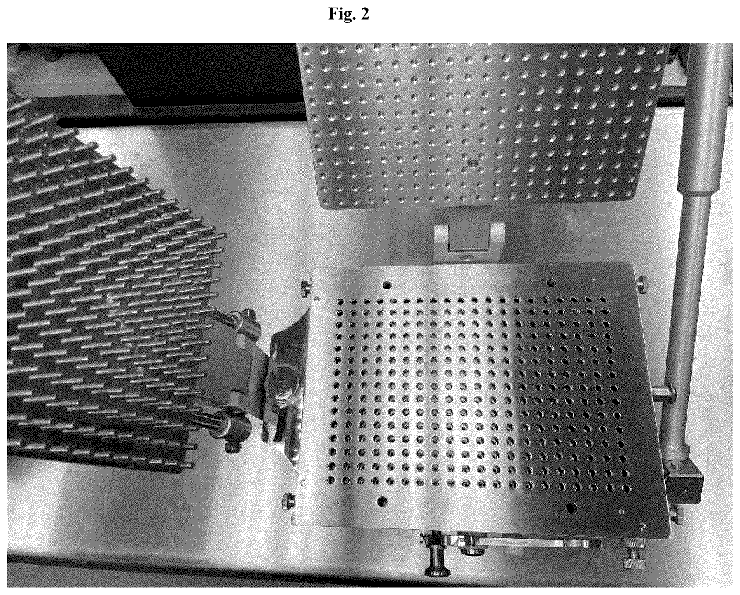

In embodiments, capsules comprising Cannabinoid Products described herein are created by filling the capsule shell with a Cannabinoid Product. In embodiments, a capsule filling machine is used to fill the capsule shells. For example, in embodiments, the capsules comprising the Cannabinoid Products described herein are created with the capsule filling machine of FIG. 2.

### Diabetes

The most common types of diabetes mellitus are Type I and Type II. In Type I diabetes, the beta cells in the pancreas, often through an auto-immune reaction, cease secreting insulin into the bloodstream of the person. Insulin is a chemical substance which is normally secreted into the bloodstream by beta cells within the pancreas. Insulin is vitally important to the person because it enables the person to properly utilize and regulate sugar in the bloodstream as part of the metabolism process.

Type II diabetes, or non-insulin-independent diabetes, often occurs in the face of normal, or even elevated levels of insulin and appears to be the result of the inability of tissues to respond appropriately to insulin. Most of the Type II diabetics are also obese. In Type I cases, where the pancreas has ceased producing insulin, it is necessary for the afflicted person to inject insulin at prescribed periodic intervals and dosages in order to control the level of sugar in the blood. Oral ingestion of insulin is also possible but usually less effective due to the degradation of insulin caused by the passage through the stomach and upper intestine.

In Type II diabetes, the pancreas continues to produce insulin but, some or all of the insulin may fail to bind to the body cell receptors and/or internalization of insulin in the cells is reduced. In such cases, there may be a sufficient level of insulin in the blood, but the ability of the cells to uptake glucose is reduced or non-existent because of reduced internalized insulin. When a Type II diabetic cell binds insulin but does not take up glucose, it indicates a defect in the signaling pathway. This results in an increased need for insulin; however, this need for insulin is not met because the R cells in a Type II diabetic are defective in that they do not secrete enough insulin.

The existence of Type I or Type II diabetes in a person is usually determined by an oral glucose tolerance test (OGTT). OGTT is a test in which the fasting patient is given a known amount of glucose (sugar) by mouth, and the blood is tested at intervals thereafter to note the quantity of sugar in the blood. A curve is then constructed from which important information about the person can be drawn. The glucose tolerance test curve will typically show whether the patient is hyperglycaemic (diabetic) or whether the patient has too little sugar in his or her blood and is therefore hypoglycaemic.

Symptoms of hyperglycaemia can be headaches, increased urination, thirst, nausea, weight loss, fatigue and coma. Hyperglycaemia can be caused by Hypoinsulinism, a condition in which the insulin producing beta cells of the pancreas fail to manufacture insulin or manufacture arid secrete a reduced amount of insulin into the bloodstream. In such cases, levels of sugar in the blood are dramatically increased.

Type II diabetes is a progressive, multifactorial disease which results from insulin resistance and is characterized initially by elevated fasting blood glucose levels. It is believed that genetic factors contribute to susceptibility to type II diabetes, but other important risk factors such as, obesity, aging, diet, and lack of exercise also play a role.

### Peripheral Neuropathy

Peripheral neuropathy is the most common type of diabetic neuropathy. Peripheral neuropathy affects the feet and legs first, followed by the hands and arms. Signs and symptoms of peripheral neuropathy are often worse at night, and may include:
Numbness or reduced ability to feel pain or temperature changes;
Tingling or burning sensation;
Sharp pains or cramps;
Increased sensitivity to touch, and/or,
Serious foot problems, such as ulcers, infections, and bone and joint pain.

The exact cause of each type of neuropathy is unknown. Researchers believe that, over time, uncontrolled high blood sugar damages nerves and interferes with their ability to send signals, leading to diabetic neuropathy. High blood sugar also weakens the walls of the small blood vessels (capillaries) that supply the nerves with oxygen and nutrients.

### Pain

Subjects with diabetes often have different forms of pain. Pain can be generally classified to two broad categories, acute and chronic. The treatment of any acute or chronic pain is subject matter of the present disclosure. Acute pain is usually associated with a specific cause such as a specific injury and is often sharp and severe. Acute pain begins suddenly and is not persistent. Chronic pain is long-term pain, with a typical duration of more than three months leading to significant psychological and emotional problems. Chronic pain is generally associated with clinical conditions characterized by chronic and/or degenerative lesions. Common examples of chronic pain are neuropathic pain, such as painful diabetic neuropathy.

Diabetes and related metabolic disorders are a common cause of peripheral neuropathic pain (diabetic neuropathy). Diabetic peripheral neuropathy (DPN) is a particularly debilitating complication of diabetes resulting from sensory and motor neuron damage. Up to half of diabetic patients have some degree of DPN. Symptoms are typically dominated by sensory defects. Early in the disease process, DPN is manifested by hyperalgesia, but over time patients suffer from become hyposensitive, patients suffer from hypoalgesia and muscle weakness. This hyposensitivity can lead to significant morbidity by predisposing the lower extremities to injury, ulceration and eventual amputation. There are treatment options for pain relief for the early hyperalgesia stage of DPN. However, currently, there is no treatment for this later hypoalgesia, or hyposensitivity, stage.

Common features of DPN may include:
Nerve damage;
Numbness in the hands or feet; and/or,
Pain in the hands, feet, or legs.

Subjects may also have problems with their digestive tract, heart, sex organs, or their eyes. Depending on the affected nerves, diabetic neuropathy symptoms can range from pain and numbness in the legs and feet to problems with the digestive system, urinary tract, blood vessels and heart.

### Measurements of Pain in Dilated Peripheral Neuropathy

According to the present disclosure, pain and conditions associated with pain (e.g. itching or depression) can be treated, prevented, improved, and/or reduced. “Preventing” or “prevention” herein does not require absolute success in the sense of an absolute prevention of pain but indicates a reduced risk of developing a disease or painful condition, or developing pain with reduced severity. Likewise, “treatment” shall not be construed as an absolute cure, but may also relate to amelioration or suppression of pain or pain associated conditions. A person of ordinary skill in the art would understand that pain “improvement” and “reduction” both refer to an overall decrease in the symptoms associated with DPN. Similarly, “improvement” and “reduction” shall not be construed as an absolute cure, but may also relate to amelioration or suppression of pain or pain associated conditions.

In some embodiments, a formulation of the present disclosure reduces symptoms of neuropathic pain in a subject with diabetes.

Pain and pain associated conditions and diseases to be treated according to the present disclosure can include acute pain, chronic pain, somatogenic pain, neuropathic pain, psychogenic pain, heat induced pain, physical pain and nociception in general, or hyperalgesia. In some embodiments, the pain is from neuropathic pain, in both acute and chronic forms. The pain can also be related to phantom pain, pain from a part of the body that has been lost or from which the brain no longer receives physical signals.

In some embodiments, the formulation, tablet, and/or capsule is administered in a dosage sufficient to treat or prevent pain or associated conditions and diseases.

In some embodiments, a formulation, tablet, and/or capsule are used to treat pain associated with DPN.

Administration can e.g. be a single dose administration or a successive or repeated administration, e.g. twice a day, daily or in an interval of at least 1 day, at least 2 days, at least 3 days, at least 1 week, at least 2 weeks, at least 4 weeks, at least 8 weeks or even more preferred at least 12 weeks, including any interval in between.

In some embodiments, administration of the formulation, tablet, and/or capsule are administered at least once a day, at least twice a day, at least three times a day, at least four times a day, at least five times a day, at least six times a date, at least seven times a day, at least eight times a day, at least nine times a day, or at least ten times a day, including any interval in between.

In some embodiments, administration of the formulation, tablet, and/or capsule are administered at least once per week, at least twice per week, at least three times per week, at least four times a week, at least five times a week, at least six times a week, or seven days a week, including any interval in between.

In some embodiments, administration of the formulation, tablet, and/or capsule occurs for at least 1 day, at least 1 week, at least 1 month, at least 2 months, at least 3 months, at least 4 months, at least 5 months, at least 6 months, at least 12 months, at least 24 months, at least 36 months, at least 48 months, at least 60 months, at least 72 months, at least 84 months, at least 96 months, at least 108 months, or at least 120 months, including any interval in between.

In some embodiments, administration of the formulation, tablet, and/or capsule occurs for at least 1 day, at least 1 week, at least 1 month, at least 2 months, at least 3 months, at least 4 months, at least 5 months, at least 6 months, at least 12 months, at least 24 months, at least 36 months, at least 48 months, at least 60 months, at least 72 months, at least 84 months, at least 96 months, at least 108 months, or at least 120 months, including any interval in between.

In some embodiments, the formulation, tablet, and/or capsule is administered to prevent pain. Preventive administrations are usually a short time before expected pain, if controllable or foreseeable—such as in scheduled surgery—e.g. up to at least 1 hour, at least 2 hours, at least 3 hours, at least 4 hours, at least 5 hours, at least 6 hours, at least 8 hours, at least 10 hours, at least 12 hours, at least 24 hours, or at least 48 hours beforehand, including any interval in between. In some embodiments, the formulation, tablet, and/or capsule is administered in response to pain.

Persons having skill in the art will be familiar with the multiple ways of assessing pain in patients. In some embodiments, the symptoms of neuropathic pain may be evaluated using the Numerical Rating Scale (NRS) (see Hawker et al., Arthritis Care Res (Hoboken), 63(11):240-52, 2011), the Neuropathic Pain Symptom Inventory (NPSI) (see Bouhassira et al. Pain. 108(3):248-57, 2004), the short-form McGill Pain Questionnaire (SF-MPQ) (see Waldman, Pain Review, 2009), the Visual Analogue Scale (VAS) (see Duarte et al. Qual Life Res. 25(7):1771-7, 2016), the Daily Sleep Interference Scale Score (DSIS) (see Vernon et al., J Pain Symptom Manage 36(1):54-68, 2008), the Clinical Global Impression of Change (CGIC) (see Busner et al., Pshychiatry 4(7):28-37, 2007), the Patient Global Impression of Change (PGIC) (see Ferguson et al., Journal of Pain 10(4)), Hospital Anxiety and Depression Scale (HADS) (see Stern et al., Occupational Medicine, 64(5):393-94, 2014), a change in weight, a change in total activity, a change in use of rescue medication, a change in the occurrence of loss of balance, a change in sensory issues, and/or a change in muscular issues, all of which are incorporated by reference in their entirety.

In some embodiments, the formulation, tablet, and/or capsule of the present disclosure reduces pain as measured by the NRS. In some embodiments, the pain is reduced by at least 1%, at least 5%, at least 10%, at least 20%, at least 30%, at least 40%, at least 50%, at least 60%, at least 70%, at least 80%, at least 90%, or 100%, as well as any interval in between, as measured by the NRS compared to a subject that is not administered the formulation, tablet, and/or capsule of the present disclosure.

In some embodiments, the pain is reduced by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the NRS compared to a subject that is not administered the formulation, tablet, and/or capsule of the present disclosure.

In some embodiments, the formulation, tablet, and/or capsule of the present disclosure reduces pain as measured by the NPSI. In some embodiments, the pain is reduced by at least 1%, at least 5%, at least 10%, at least 20%, at least 30%, at least 40%, at least 50%, at least 60%, at least 70%, at least 80%, at least 90%, or 100%, as well as any interval in between, as measured by the NPSI compared to a subject that is not administered the formulation, tablet, and/or capsule of the present disclosure.

In some embodiments, the pain is reduced by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the NPSI compared to a subject that is not administered the formulation, tablet, and/or capsule of the present disclosure.

In some embodiments, the formulation, tablet, and/or capsule of the present disclosure reduces pain as measured by the SF-MPQ. In some embodiments, the pain is reduced by at least 1%, at least 5%, at least 10%, at least 20%, at least 30%, at least 40%, at least 50%, at least 60%, at least 70%, at least 80%, at least 90%, or 100%, as well as any interval in between, as measured by the SF-MPQ compared to a subject that is not administered the formulation, tablet, and/or capsule of the present disclosure. In some embodiments the pain score is the sensory score. In some embodiments the pain score is the affective score.

In some embodiments, the pain is reduced by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the SF-MPQ compared to a subject that is not administered the formulation, tablet, and/or capsule of the present disclosure.

In some embodiments, the formulation, tablet, and/or capsule of the present disclosure reduces pain as measured by the VAS. In some embodiments, the pain is reduced by at least 1%, at least 5%, at least 10%, at least 20%, at least 30%, at least 40%, at least 50%, at least 60%, at least 70%, at least 80%, at least 90%, or 100%, as well as any interval in between, as measured by the VAS compared to a subject that is not administered the formulation, tablet, and/or capsule of the present disclosure.

In some embodiments, the pain is reduced by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the VAS compared to a subject that is not administered the formulation, tablet, and/or capsule of the present disclosure.

In some embodiments, the formulation, tablet, and/or capsule of the present disclosure reduces pain as measured by the DSIS. In some embodiments, the pain is reduced by at least 1%, at least 5%, at least 10%, at least 20%, at least 30%, at least 40%, at least 50%, at least 60%, at least 70%, at least 80%, at least 90%, or 100%, as well as any interval in between, as measured by the DSIS compared to a subject that is not administered the formulation, tablet, and/or capsule of the present disclosure.

In some embodiments, the pain is reduced by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the DSIS compared to a subject that is not administered the formulation, tablet, and/or capsule of the present disclosure.

In some embodiments, the formulation, tablet, and/or capsule of the present disclosure reduces pain as measured by the CGIC. In some embodiments, the pain is reduced by at least 1%, at least 5%, at least 10%, at least 20%, at least 30%, at least 40%, at least 50%, at least 60%, at least 70%, at least 80%, at least 90%, or 100%, as well as any interval in between, as measured by the CGIC compared to a subject that is not administered the formulation, tablet, and/or capsule of the present disclosure.

In some embodiments, the pain is reduced by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the CGIC compared to a subject that is not administered the formulation, tablet, and/or capsule of the present disclosure.

In some embodiments, the formulation, tablet, and/or capsule of the present disclosure reduces pain as measured by the PGIC. In some embodiments, the pain is reduced by at least 1%, at least 5%, at least 10%, at least 20%, at least 30%, at least 40%, at least 50%, at least 60%, at least 70%, at least 80%, at least 90%, or 100%, as well as any interval in between, as measured by the PGIC compared to a subject that is not administered the formulation, tablet, and/or capsule of the present disclosure.

In some embodiments, the pain is reduced by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the PGIC compared to a subject that is not administered the formulation, tablet, and/or capsule of the present disclosure.

In some embodiments, the formulation, tablet, and/or capsule of the present disclosure reduces pain as measured by the HADS. In some embodiments, the pain is reduced by at least 1%, at least 5%, at least 10%, at least 20%, at least 30%, at least 40%, at least 50%, at least 60%, at least 70%, at least 80%, at least 90%, or 100%, as well as any interval in between, as measured by the HADS compared to a subject that is not administered the formulation, tablet, and/or capsule of the present disclosure.

In some embodiments, the pain is reduced by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the HADS compared to a subject that is not administered the formulation, tablet, and/or capsule of the present disclosure.

In some embodiments, a reduction in pain results in greater mobility in the subject. In some embodiments, the greater mobility leads to a change in weight. In some embodiments, the formulation, tablet, and/or capsule of the present disclosure reduces pain as measured by a change in weight. For example, a subject with less pain may increase mobility that causes a change in weight from 200 pounds to 180 pounds, or a 10% reduction in weight. In some embodiments, the weight is reduced by at least 1%, at least 5%, at least 10%, at least 20%, at least 30%, at least 40%, at least 50%, or at least 60%, as well as any interval in between, as measured by the subject's weight compared to a subject that is not administered the formulation, tablet, and/or capsule of the present disclosure.

In some embodiments, the weight is reduced by between 1% to 5%, between 5% to 10%, between 10% to 20%, between 20% to 30%, between 30% to 40%, between 40% to 50%, or between 50% to 60%, as well as any interval in between, as measured by the subject's weight compared to a subject that is not administered the formulation, tablet, and/or capsule of the present disclosure.

In some embodiments, a reduction in pain results in greater mobility in the subject. In some embodiments, the formulation, tablet, and/or capsule of the present disclosure reduces pain as measured by total activity. Total activity per day reflects the total amount of activity across all intensities. For example, a subject with less pain may increase daily activity from 20 minutes to 30 minutes, or a 50% increase in total activity. In some embodiments, the total activity is measured daily, weekly, monthly, and/or yearly. In some embodiments, the total activity is increased by at least 1%, at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 750%, or at least 1000%, as well as any interval in between, as measured by the total activity compared to a subject that is not administered the formulation, tablet, and/or capsule of the present disclosure.

In some embodiments, the total activity is increased by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, between 90% to 100%, between 100% to 200%, between 200% to 300%, between 300% to 400%, between 400% to 500%, between 500% to 750%, or between 750% to 1000%, as well as any interval in between, as measured by the total activity compared to a subject that is not administered the formulation, tablet, and/or capsule of the present disclosure.

In some embodiments, a reduction in pain results in using less rescue medications. In some embodiments, rescue medications are opioid medications. In some embodiments, the formulation, tablet, and/or capsule of the present disclosure reduces pain as measured by use of rescue medications. For example, a subject with less pain may decrease rescue medication use from 5 rescue medications a week to minutes to 1 rescue medication per week, or an 80% reduction in rescue medication use per week. In some embodiments, the rescue medication use is measured daily, weekly, monthly, and/or yearly. In some embodiments, the total activity is decreased by at least 1%, at least 5%, at least 10%, at least 20%, at least 30%, at least 40%, at least 50%, at least 60%, at least 70%, at least 80%, at least 90%, at least 100%, as well as any interval in between, as measured by the rescue medication use compared to a subject that is not administered the formulation, tablet, and/or capsule of the present disclosure.

In some embodiments, the rescue medication use is decreased by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by rescue medication use compared to a subject that is not administered the formulation, tablet, and/or capsule of the present disclosure.

In some embodiments, a reduction in pain results in a reduction of loss of balance incidents. In some embodiments, diabetic neuropathy pain causes a loss of balance. Loss of balance may include, but is not limited to, a feeling of unsteadiness or dizziness while sitting or walking, and/or falling while getting up, standing, or walking. In some embodiments, the formulation, tablet, and/or capsule of the present disclosure reduces pain as measured by the number of by loss of balance incidents. For example, a subject with less pain may decrease the number of times balance is lost from 10 incidents a week to 1 incident per week, or a 90% reduction in loss of balance incidents per week. In some embodiments, the loss of balance incidents are measured daily, weekly, monthly, and/or yearly. In some embodiments, the loss of balance incidents are decreased by at least 1%, at least 5%, at least 10%, at least 20%, at least 30%, at least 40%, at least 50%, at least 60%, at least 70%, at least 80%, at least 90%, or 100%, as well as any interval in between, as measured by the number of loss of balance incidents compared to a subject that is not administered the formulation, tablet, and/or capsule of the present disclosure.

In some embodiments, the loss of balance incidents are decreased by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the number of loss of balance incidents compared to a subject that is not administered the formulation, tablet, and/or capsule of the present disclosure.

In some embodiments, a reduction in pain results in a reduction of sensory issue incidents. Sensory issues may include, but is not limited to, a sensation of pins and needles, uncomfortable tingling and burning, oversensitivity, reduced sensation of touch, or sensitivity to pain. In some embodiments, the formulation, tablet, and/or capsule of the present disclosure reduces pain as measured by sensory issue incidents. For example, a subject with less pain may decrease the number of times sensory issue incidents occur from 10 incidents a week to 1 incident per week, or a 90% reduction in sensory issue incidents per week. In some embodiments, the sensory issue incidents are measured daily, weekly, monthly, and/or yearly. In some embodiments, the sensory issue incidents are decreased by at least 1%, at least 5%, at least 10%, at least 20%, at least 30%, at least 40%, at least 50%, at least 60%, at least 70%, at least 80%, at least 90%, or 100%, as well as any interval in between, as measured by the number of sensory issue incidents compared to a subject that is not administered the formulation, tablet, and/or capsule of the present disclosure.

In some embodiments, the sensory issue incidents are decreased by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the number of sensory issue incidents compared to a subject that is not administered the formulation, tablet, and/or capsule of the present disclosure.

In some embodiments, a reduction in pain results in a reduction of muscular issue incidents. Muscular issues may include, but are not limited to, cramping, muscle weakness or problems with coordination. In some embodiments, the formulation, tablet, and/or capsule of the present disclosure reduces pain as measured by muscular issue incidents. For example, a subject with less pain may decrease the number of times muscular issue incidents occur from 10 incidents a week to 1 incident per week, or a 90% reduction in muscular issue incidents per week. In some embodiments, the muscular issue incidents are measured daily, weekly, monthly, and/or yearly. In some embodiments, the muscular issue incidents are decreased by at least 1%, at least 5%, at least 10%, at least 20%, at least 30%, at least 40%, at least 50%, at least 60%, at least 70%, at least 80%, at least 90%, or 100%, as well as any interval in between, as measured by the number of muscular issue incidents compared to a subject that is not administered the formulation, tablet, and/or capsule of the present disclosure.

In some embodiments, the muscular issue incidents are decreased by between 1% to 5%, between 5% to 10%, between 10% to 20%, between 20% to 30%, between 30% to 40%, between 40% to 50%, between 50% to 60%, between 60% to 70%, between 70% to 80%, between 80% to 90%, or between 90% to 100%, as well as any interval in between, as measured by the number of muscular issue incidents compared to a subject that is not administered the formulation, tablet, and/or capsule of the present disclosure.

### Pharmaceutical Approaches to Treating Diabetic Neuropathy

Diabetic neuropathy treatment depends on the subject's symptoms, age, and general health. Diabetic neuropathy treatment also depends on condition severity.

The main goal of treatment is to ease pain and discomfort. Treatment can also help prevent more tissue damage.

In some embodiments, a formulation of the present disclosure may be administered as sole treatment or as part of a combination treatment comprising a secondary treatment selected from the group consisting of:
Anti-seizure medications to reduce pain;
Gabapentin;
Pregabalin;
Antidepressants for relief from mild to moderate symptoms;
Topical creams;
Transcutaneous electronic nerve stimulation (TENS) therapy;
Hypnosis;
Relaxation training;
Biofeedback;
Acupuncture; and/or,
Special shoes to protect feet from injuries

There are treatment options for pain relief for the early hyperalgesia stage of DPN. These treatment options include general pain relief medications, such as Tylenol, but may also include opiates for more severe cases. Additionally, there is no treatment for later hypoalgesia, or hyposensitivity, stages. Consequently, new treatments must be developed to better treat all stages of DPN.

### ZLT-L-007 Formulation

The formulation of the present disclosure, ZLT-L-007, was developed to treat pain associated with DPN at all stages. In some embodiments, ZLT-L-007 provides pain relief to subjects with DPN. In some embodiments, ZLT-L-007 provides pain relief to subjects for the early hyperalgesia stage of DPN. In some embodiments, ZLT-L-007 provides pain relief to subjects for the later hypoalgesia stage of DPN.

In some embodiments, the ZLT-L-007 formulation comprises Cannabidiol (CBD), Cannabigerol (CBG), Δ8-tetrahydrocannabinol (D8THC), a non-active excipient, sodium stearyl fumarate, and a terpene component.

In some embodiments, the non-active excipient comprises colloidal silicon dioxide, sodium starch glycolate, sodium stearyl fumarate and/or mannitol.

In some embodiments, the terpene component comprises myrcene terpene, limonene terpene, linalool terpene, caryophyllene terpene, alpha-pinene terpene, beta-pinene terpene, alpha-bisabolol terpene, eucalyptol terpene, trans-nerolido terpene, humulene terpene, A3-carene terpene, camphene terpene, borneol terpene, terpineol terpene, valencene terpene, and/or geraniol terpene.

In some embodiments, the ZLT-L-007 formulation comprises Cannabidiol (CBD), Cannabigerol (CBG), Δ8-tetrahydrocannabinol (D8THC), a non-active excipient, sodium stearyl fumarate, and linalool terpene.

In some embodiments, the ZLT-L-007 formulation comprises Cannabidiol (CBD), Cannabigerol (CBG), Δ8-tetrahydrocannabinol (D8THC), a non-active excipient, sodium stearyl fumarate, and myrcene terpene.

In some embodiments, the ZLT-L-007 formulation comprises Cannabidiol (CBD), Cannabigerol (CBG), Δ8-tetrahydrocannabinol (D8THC), a non-active excipient, sodium stearyl fumarate, myrcene terpene, and linalool terpene.

In some embodiments, the ZLT-L-007 formulation comprises Cannabidiol (CBD), Cannabigerol (CBG), Δ8-tetrahydrocannabinol (D8THC), colloidal silicon dioxide, sodium stearyl fumarate, and linalool terpene.

In some embodiments, the ZLT-L-007 formulation comprises Cannabidiol (CBD), Cannabigerol (CBG), Δ8-tetrahydrocannabinol (D8THC), colloidal silicon dioxide, sodium stearyl fumarate, and myrcene terpene.

In some embodiments, the ZLT-L-007 formulation comprises Cannabidiol (CBD), Cannabigerol (CBG), Δ8-tetrahydrocannabinol (D8THC), colloidal silicon dioxide, sodium stearyl fumarate, myrcene terpene, and linalool terpene.

In some embodiments, the ZLT-L-007 formulation comprises at least 10% (w/w), at least 20% (w/w), at least 30% (w/w), at least 40% (w/w), at least 50% (w/w), at least 60% (w/w), at least 70% (w/w), or at least 80% (w/w) CBD.

In some embodiments, the ZLT-L-007 formulation comprises about 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80 (w/w) CBD, including all ranges and subranges therebetween.

In some embodiments, the ZLT-L-007 formulation comprises about 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80 (w/w) CBD, by weight of the cannabinoid fraction, including all ranges and subranges therebetween. In some embodiments the cannabinoid fraction is the additive weight of all the cannabinoids in the formulation.

In some embodiments, the ZLT-L-007 formulation comprises at least 10 mg, at least 20 mg, at least 30 mg, at least 40 mg, at least 50 mg, at least 60 mg, at least 70 mg, at least 80 mg, at least 90 mg, at least 100 mg, at least 200 mg, at least 300 mg, at least 400 mg, at least 500 mg, or at least 1000 mg CBD.

In some embodiments, the ZLT-L-007 formulation comprises between 10 mg to 20 mg, between 20 mg to 30 mg, between 30 mg to 40 mg, between 40 mg to 50 mg, between 50 mg to 60 mg, between 60 mg to 70 mg, between 70 mg to 80 mg, between 80 mg to 90 mg, between 90 mg to 100 mg, between 100 mg to 200 mg, between 200 mg to 300 mg, between 300 mg to 400 mg, between 400 mg to 500 mg, or between 500 mg to 1000 mg CBD, including all ranges and subranges therebetween.

In some embodiments, the ZLT-L-007 formulation comprises about 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80 (w/w) CBG, including all ranges and subranges therebetween.

In some embodiments, the ZLT-L-007 formulation comprises about 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80 (w/w) CBG, by weight of the cannabinoid fraction, including all ranges and subranges therebetween. In some embodiments the cannabinoid fraction is the additive weight of all the cannabinoids in the formulation.

In some embodiments, the ZLT-L-007 formulation comprises at least 0.1 mg, at least 1 mg, at least 10 mg, at least 20 mg, at least 30 mg, at least 40 mg, at least 50 mg, at least 60 mg, at least 70 mg, at least 80 mg, at least 90 mg, at least 100 mg, at least 200 mg, at least 300 mg, at least 400 mg, at least 500 mg, or at least 1000 mg CBG.

In some embodiments, the ZLT-L-007 formulation comprises between 0.1 mg to 1 mg, between 1 mg to 10 mg, between 10 mg to 20 mg, between 20 mg to 30 mg, between 30 mg to 40 mg, between 40 mg to 50 mg, between 50 mg to 60 mg, between 60 mg to 70 mg, between 70 mg to 80 mg, between 80 mg to 90 mg, between 90 mg to 100 mg, between 100 mg to 200 mg, between 200 mg to 300 mg, between 300 mg to 400 mg, between 400 mg to 500 mg, or between 500 mg to 1000 mg CBG.

In some embodiments, the ZLT-L-007 formulation comprises about 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80 (w/w) Δ8-THC, including all ranges and subranges therebetween.

In some embodiments, the ZLT-L-007 formulation comprises about 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80 (w/w) Δ8-THC, by weight of the cannabinoid fraction, including all ranges and subranges therebetween. In some embodiments the cannabinoid fraction is the additive weight of all the cannabinoids in the formulation.

In some embodiments, the ZLT-L-007 formulation comprises at least 0.1 mg, at least 1 mg, at least 10 mg, at least 20 mg, at least 30 mg, at least 40 mg, at least 50 mg, at least 60 mg, at least 70 mg, at least 80 mg, at least 90 mg, at least 100 mg, at least 200 mg, at least 300 mg, at least 400 mg, at least 500 mg, or at least 1000 mg Δ8-THC.

In some embodiments, the ZLT-L-007 formulation comprises between 0.1 mg to 1 mg, between 1 mg to 10 mg, between 10 mg to 20 mg, between 20 mg to 30 mg, between 30 mg to 40 mg, between 40 mg to 50 mg, between 50 mg to 60 mg, between 60 mg to 70 mg, between 70 mg to 80 mg, between 80 mg to 90 mg, between 90 mg to 100 mg, between 100 mg to 200 mg, between 200 mg to 300 mg, between 300 mg to 400 mg, between 400 mg to 500 mg, or between 500 mg to 1000 mg Δ8-THC, including all ranges and subranges therebetween.

In some embodiments, the ZLT-L-007 formulation comprises about 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80 (w/w) colloidal silicon dioxide, including all ranges and subranges therebetween.

In some embodiments, the ZLT-L-007 formulation comprises at least 0.1 mg, at least 1 mg, at least 10 mg, at least 20 mg, at least 30 mg, at least 40 mg, at least 50 mg, at least 60 mg, at least 70 mg, at least 80 mg, at least 90 mg, at least 100 mg, at least 200 mg, at least 300 mg, at least 400 mg, at least 500 mg, or at least 1000 mg colloidal silicon dioxide.

In some embodiments, the ZLT-L-007 formulation comprises between 0.1 mg to 1 mg, between 1 mg to 10 mg, between 10 mg to 20 mg, between 20 mg to 30 mg, between 30 mg to 40 mg, between 40 mg to 50 mg, between 50 mg to 60 mg, between 60 mg to 70 mg, between 70 mg to 80 mg, between 80 mg to 90 mg, between 90 mg to 100 mg, between 100 mg to 200 mg, between 200 mg to 300 mg, between 300 mg to 400 mg, between 400 mg to 500 mg, or between 500 mg to 1000 mg colloidal silicon dioxide, including all ranges and subranges therebetween.

In some embodiments, the ZLT-L-007 formulation comprises 0.01% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80 (w/w) sodium stearyl fumarate, including all ranges and subranges therebetween.

In some embodiments, the ZLT-L-007 formulation comprises at least 0.01 mg, at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2.0 mg, at least 2.1 mg, at least 2.2 mg, at least 2.3 mg, at least 2.4 mg, at least 2.5 mg, at least 2.6 mg, at least 2.7 mg, at least 2.8 mg, at least 2.9 mg, at least 3.0 mg, at least 5.0 mg, or at least 10.0 mg sodium stearyl fumarate.

In some embodiments, the ZLT-L-007 formulation comprises between 0.01 mg to 0.1 mg, between 0.1 mg to 0.2 mg, between 0.2 mg to 0.3 mg, between 0.3 mg to 0.4 mg, between 0.4 mg to 0.5 mg, between 0.5 mg to 0.6 mg, between 0.6 mg to 0.7 mg, between 0.7 mg to 0.8 mg, between 0.8 mg to 0.9 mg, between 0.9 mg to 1.0 mg, between 1.0 mg to 1.1 mg, between 1.1 mg to 1.2 mg, between 1.2 mg to 1.3 mg, between 1.3 mg to 1.4 mg, between 1.4 mg to 1.5 mg, between 1.5 mg to 1.6 mg, between 1.6 mg to 1.7 mg, between 1.7 mg to 1.8 mg, between 1.8 mg to 1.9 mg, between 1.9 mg to 2.0 mg, between 2.0 mg to 2.1 mg, between 2.1 mg to 2.2 mg, between 2.2 mg to 2.3 mg, between 2.3 mg to 2.4 mg, between 2.4 mg to 2.5 mg, between 2.5 mg to 2.6 mg, between 2.6 mg to 2.7 mg, between 2.7 mg to 2.8 mg, between 2.8 mg to 2.9 mg, between 2.9 mg to 3.0 mg, between 3.0 mg to 5.0 mg, or between 5.0 mg to 10.0 mg sodium stearyl fumarate, including all ranges and subranges therebetween.

In some embodiments, the ZLT-L-007 formulation comprises 0.01% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80% (w/w) terpene component, including all ranges and subranges therebetween.

In some embodiments, the ZLT-L-007 formulation comprises at least 0.01 mg, at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2.0 mg, at least 2.1 mg, at least 2.2 mg, at least 2.3 mg, at least 2.4 mg, at least 2.5 mg, at least 2.6 mg, at least 2.7 mg, at least 2.8 mg, at least 2.9 mg, at least 3.0 mg, at least 5.0 mg, or at least 10.0 mg terpene component.

In some embodiments, the ZLT-L-007 formulation comprises between 0.01 mg to 0.1 mg, between 0.1 mg to 0.2 mg, between 0.2 mg to 0.3 mg, between 0.3 mg to 0.4 mg, between 0.4 mg to 0.5 mg, between 0.5 mg to 0.6 mg, between 0.6 mg to 0.7 mg, between 0.7 mg to 0.8 mg, between 0.8 mg to 0.9 mg, between 0.9 mg to 1.0 mg, between 1.0 mg to 1.1 mg, between 1.1 mg to 1.2 mg, between 1.2 mg to 1.3 mg, between 1.3 mg to 1.4 mg, between 1.4 mg to 1.5 mg, between 1.5 mg to 1.6 mg, between 1.6 mg to 1.7 mg, between 1.7 mg to 1.8 mg, between 1.8 mg to 1.9 mg, between 1.9 mg to 2.0 mg, between 2.0 mg to 2.1 mg, between 2.1 mg to 2.2 mg, between 2.2 mg to 2.3 mg, between 2.3 mg to 2.4 mg, between 2.4 mg to 2.5 mg, between 2.5 mg to 2.6 mg, between 2.6 mg to 2.7 mg, between 2.7 mg to 2.8 mg, between 2.8 mg to 2.9 mg, between 2.9 mg to 3.0 mg, between 3.0 mg to 5.0 mg, or between 5.0 mg to 10.0 mg terpene component, including all ranges and subranges therebetween.

In some embodiments, the ZLT-L-007 formulation comprises 0.01% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80 (w/w) myrcene terpene, including all ranges and subranges therebetween.

In some embodiments, the ZLT-L-007 formulation comprises at least 0.01 mg, at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2.0 mg, at least 2.1 mg, at least 2.2 mg, at least 2.3 mg, at least 2.4 mg, at least 2.5 mg, at least 2.6 mg, at least 2.7 mg, at least 2.8 mg, at least 2.9 mg, at least 3.0 mg, at least 5.0 mg, or at least 10.0 mg myrcene terpene.

In some embodiments, the ZLT-L-007 formulation comprises between 0.01 mg to 0.1 mg, between 0.1 mg to 0.2 mg, between 0.2 mg to 0.3 mg, between 0.3 mg to 0.4 mg, between 0.4 mg to 0.5 mg, between 0.5 mg to 0.6 mg, between 0.6 mg to 0.7 mg, between 0.7 mg to 0.8 mg, between 0.8 mg to 0.9 mg, between 0.9 mg to 1.0 mg, between 1.0 mg to 1.1 mg, between 1.1 mg to 1.2 mg, between 1.2 mg to 1.3 mg, between 1.3 mg to 1.4 mg, between 1.4 mg to 1.5 mg, between 1.5 mg to 1.6 mg, between 1.6 mg to 1.7 mg, between 1.7 mg to 1.8 mg, between 1.8 mg to 1.9 mg, between 1.9 mg to 2.0 mg, between 2.0 mg to 2.1 mg, between 2.1 mg to 2.2 mg, between 2.2 mg to 2.3 mg, between 2.3 mg to 2.4 mg, between 2.4 mg to 2.5 mg, between 2.5 mg to 2.6 mg, between 2.6 mg to 2.7 mg, between 2.7 mg to 2.8 mg, between 2.8 mg to 2.9 mg, between 2.9 mg to 3.0 mg, between 3.0 mg to 5.0 mg, or between 5.0 mg to 10.0 mg myrcene terpene, including all ranges and subranges therebetween.

In some embodiments, the ZLT-L-007 formulation comprises 0.01% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), or 80 (w/w) linalool terpene, including all ranges and subranges therebetween.

In some embodiments, the ZLT-L-007 formulation comprises at least 0.01 mg, at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2.0 mg, at least 2.1 mg, at least 2.2 mg, at least 2.3 mg, at least 2.4 mg, at least 2.5 mg, at least 2.6 mg, at least 2.7 mg, at least 2.8 mg, at least 2.9 mg, at least 3.0 mg, at least 5.0 mg, or at least 10.0 mg linalool terpene.

In some embodiments, the ZLT-L-007 formulation comprises between 0.01 mg to 0.1 mg, between 0.1 mg to 0.2 mg, between 0.2 mg to 0.3 mg, between 0.3 mg to 0.4 mg, between 0.4 mg to 0.5 mg, between 0.5 mg to 0.6 mg, between 0.6 mg to 0.7 mg, between 0.7 mg to 0.8 mg, between 0.8 mg to 0.9 mg, between 0.9 mg to 1.0 mg, between 1.0 mg to 1.1 mg, between 1.1 mg to 1.2 mg, between 1.2 mg to 1.3 mg, between 1.3 mg to 1.4 mg, between 1.4 mg to 1.5 mg, between 1.5 mg to 1.6 mg, between 1.6 mg to 1.7 mg, between 1.7 mg to 1.8 mg, between 1.8 mg to 1.9 mg, between 1.9 mg to 2.0 mg, between 2.0 mg to 2.1 mg, between 2.1 mg to 2.2 mg, between 2.2 mg to 2.3 mg, between 2.3 mg to 2.4 mg, between 2.4 mg to 2.5 mg, between 2.5 mg to 2.6 mg, between 2.6 mg to 2.7 mg, between 2.7 mg to 2.8 mg, between 2.8 mg to 2.9 mg, between 2.9 mg to 3.0 mg, between 3.0 mg to 5.0 mg, or between 5.0 mg to 10.0 mg linalool terpene, including all ranges and subranges therebetween.

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1—Stabilizing Cannabinoids in Colloidal Silicon Dioxide

The inventors conducted an initial evaluation of colloidal silicon dioxide as a stabilizer of non-crystalline cannabinoid oil. A composition containing a non-crystalline cannabinoid oil and colloidal silicon dioxide particles was prepared and compared against a composition lacking the ingredient.

A 250 g batch of the composition containing non-crystalline cannabinoid oil and colloidal silicon dioxide particles was prepared with the ingredients listed in Table 1A.

Briefly, the cannabinoid oil was initially heated to 60° C. to lower the viscosity of the oil, and remove any crystals that may have formed during storage. The non-crystalline cannabinoid oil was added to the colloidal silicon dioxide, and the combination was homogenized using a dry, high shear mixing process. Microcrystalline cellulose and lubricant were subsequently added to the composition.

TABLE 1A
Compositions

| | Capsule containing | | Capsule lacking | |
|---|---|---|---|---|
| | colloidal | | colloidal | |
| | silicon dioxide | | silicon dioxide | |
| | | (mg/ | | (mg/ |
| | (% w/w) | capsule) | (% w/w) | capsule) |
| Non-crystalline | 21.7 | 50 | 32.2 | 50 |
| cannabinoid oil from | | | | |
| heated Cannabidiol | | | | |
| (CBD) Isolate | | | | |
| Non-crystalline | 8.5 | 19.5 | 12.5 | 19.5 |
| cannabinoid oil from | | | | |
| heated Cannabigerol | | | | |
| (CBG) Isolate | | | | |
| Non-crystalline | 2.2 | 5.0 | 3.2 | 5 |
| cannabinoid oil (broad | | | | |
| spectrum) | | | | |
| Colloidal silicon dioxide | 32.4 | 74.5 | 0 | 0 |
| Microcrystalline cellulose | 34.8 | 80.0 | 51.5 | 80 |
| Sodium stearyl fumarate | 0.4 | 1.0 | 0.6 | 1 |

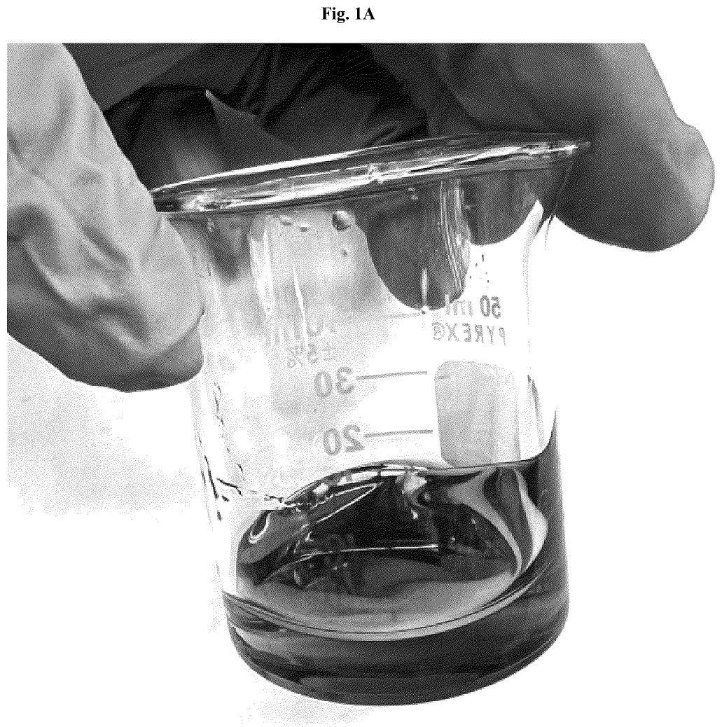

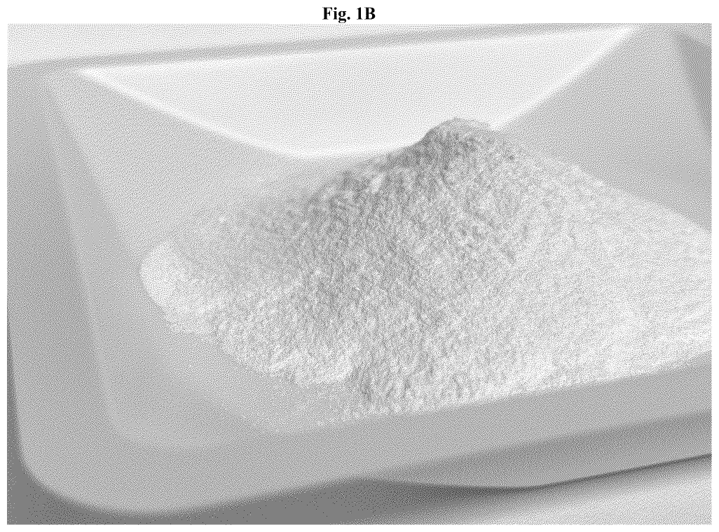

FIG. 11B shows the composition containing a non-crystalline cannabinoid oil and colloidal silicon dioxide. The composition forms a dry, non-sticky, powder, that can be dosed into pharmaceutical, homeopathic, dietary supplement, and/or recreational products. The non-crystalline cannabinoid oil is trapped in the structure of the silicon dioxide. In contrast, a composition containing a non-crystalline cannabinoid oil, but no colloidal silicon dioxide, does not form a dry powder (FIG. 1A). Rather, the non-crystalline cannabinoid oil separates from the powder bed (i.e., the CBG isolate, CBD isolate, colloidal silicon dioxide, microcrystalline cellulose, and sodium stearyl fumarate).

### Example 2—Dissolving Properties of Cannabinoid Oil and Colloidal Silicon Dioxide

Having confirmed colloidal silicon dioxide's ability to stabilize cannabinoid formulations, the inventors next turned to evaluating its dissolution properties. Capsules containing cannabinoid oil and colloidal silicon dioxide were prepared according to the recipes and methods described in Example 1. Each capsule contains 74.5 mg of active ingredients (i.e.; cannabinoid oil, including melted; CBG isolate; and melted CBD isolate). Dissolution was measured using fiber optic dissolution probes (Pion Inc.'s RAINBOW® μDiss Profiler).

Capsules were submerged in either a i) fasted state simulated intestinal fluid (FaSSIF), or ii) a fed state simulated intestinal fluid (FeSSIF). The FaSSIF contains about 3 mM taurocholate, 0.75 mM phospholipids, 148 mM sodium ions, 106 mM chloride, and 29 mM phosphate ions, thereby modeling the conditions present in fasted small intestine gastrointestinal fluid. The pH of FaSSIF is about 6.5. FeSSIF contains about 15 mM taurocholate, 3.75 mM phospholipids, 319 mM sodium ions, 203 mM chloride, and 144 mM acetic acid. The pH of FeSSIF is about 5.0. FeSSIF contains similar osmolality and pH to fed intestinal fluid. A iii) control polysorbate 80 (TWEEN® 80) solution was also tested in the experiment, to show dissolution in a simpler media. Table 2A below, summarizes the results from this experiment as percent dissolution within 60 minutes (higher value indicates greater dissolution).

TABLE 2A
Dissolution of Capsules Containing or
Lacking Colloidal Silicon Dioxide

| Formulations | Control | FaSSIF | FeSSIF |
|---|---|---|---|
| Capsules containing colloidal silicon | 36.5% | 18.1% | 42.3% |
| dioxide and non-crystalline | | | |
| cannabinoid oil | | | |
| Capsules containing non-crystalline | 8.7% | 4.1% | 17.1% |
| cannabinoid oil without colloidal | | | |
| silicon dioxide | | | |
| Dissolution Improvement Factor | 4.2X | 4.4X | 2.5X |
| (DIF) | | | |

Table 2A shows that capsules containing compositions containing colloidal silicon dioxide particles dissolved more quickly than capsules containing compositions lacking colloidal silicon dioxide particles under all conditions. Optimal dissolution was achieved by the colloidal silicon dioxide particles and non-crystalline cannabinoid oil combination under the FeSSIF. The formulation containing colloidal silicon dioxide particles however exhibited the greatest dissolution improvement factor in the FaSSIF, where the percent dissolved at 60 minutes is 4.4 times higher than oil without the colloidal silicon dioxide.

### Example 3—Tablets Containing Non-Crystalline Cannabinoid Oil

Examples 1 and 2 above demonstrated the stabilization and dissolution benefits of the combination of colloidal silicon dioxide particles and non-crystalline cannabinoid oil. This example describes the development and formulation of commercial-grade tablets. Specifically, it was discovered that additional ingredients could further improve the properties of cannabinoid-based tablets by providing more desirable hardness and dissolution properties. Assessment of each of the tested formulations is described in more detail below and in Example 4.

Tablets comprising compositions containing a non-crystalline cannabinoid oil, colloidal silicon dioxide, a filler (i.e., microcrystalline cellulose or mannitol), a disintegrant (i.e. sodium starch glycolate or croscarmellose sodium), and a lubricant (i.e. sodium stearyl fumarate) were prepared according to the methods described below. Tables 3A, 3B, and 3C show the components of each composition. Each table contains the percent by weight of each component of the compositions. Each composition in Table 3A formed a suitable tablet with desirable commercial properties. The compositions in Tables 3B and 3C did not form suitable commercial grade tablets. Tablet parameters are described in Example 4.

### Methods of Making Solid Cannabinoid Powders

Formulations K, L, M, and M[H]—Powders that Formed Suitable Tablets

Formulations K, L, and M were prepared using the following process. First, CBD oil (containing 70% CBD w/w) were mixed with ethanol and heated in an oven at 60° C. The ethanol solubilizes the CBD oil and allows for improved absorption in the colloidal silicon dioxide. Next, the heated solution of CBD oil was added to colloidal silicon dioxide gradually. Next, the mannitol and sodium starch glycolate were added. Sodium stearyl fumarate was added last. The resulting powder was compressed in a tablet press.

Formulation M[H] was prepared using the following process. First, cannabinoid oil is heated in an oven to 60° C. Next, the heated non-crystalline cannabinoid oil was added to colloidal silicon dioxide. The composition containing colloidal silicon dioxide particles and non-crystalline cannabinoid oil was mixed using a high-shear mixer (i.e., a grinder). The composition was pulsed in the grinder for about 15 to about 30 times for one second per pulse. Optionally, the composition was grinded continuously for an additional 20 seconds to homogenize the powder. Subsequently, the microcrystalline cellulose and croscarmellose sodium were added and mixed for 3 minutes in a bag, v-blender, or stand mixer. Lastly, the sodium stearyl fumarate was added and mixed for 1 minute. The short mixing time of 1 minute prevented over-blending of the sodium stearyl fumarate with the composition, which leads to worse dissolution.

Formulations L[H], K[H], J[H], I[H], H[H], G[H], A[H], B[H], C[H], D[H], E[H], and F[H]—Powders that Did not Form Suitable Commercial Grade Tablets

Formulations L[H] and K[H] were prepared according to the following method. The cannabinoid oil was heated to 60° C. and combined with colloidal silicon dioxide particles and microcrystalline cellulose. The composition containing non-crystalline cannabinoid oil and silicon dioxide was grinded for a total of 80 seconds. The croscarmellose sodium was added and the composition was blended for three minutes. Sodium stearyl fumarate was added to the composition, and the composition was blended for one minute.

Formulations J[H], I[H], and H[H] were prepared according to the following method. The cannabinoid oil was heated to 60° C. and combined with microcrystalline cellulose. The composition containing non-crystalline cannabinoid oil and microcrystalline cellulose was grinded for a total of 80 seconds. The croscarmellose sodium was added and the composition was blended for two minutes. Sodium stearyl fumarate was added to the composition, and the composition was blended for one minute.

Formulation G[H] was prepared according to the following method. The cannabinoid oil was heated to 60° C. and combined with microcrystalline cellulose. The composition containing non-crystalline cannabinoid oil and microcrystalline cellulose was grinded for a total of 60 seconds. The croscarmellose sodium was added and the composition was blended for two minutes. Sodium stearyl fumarate was added to the composition, and the composition was blended for one minute.

Formulations A[H], B[H], C[H], D[H], and F[H] were produced according to the following process. First, powdered CBD isolate, powdered CBG isolate, and cannabinoid oil were grinded with a mortar and pestle. Second, the composition containing cannabinoids was added to the colloidal silicon dioxide and grinded. Next, the composition containing cannabinoids and colloidal silicon dioxide was blended for five minutes with silicified microcrystalline cellulose, sodium starch glycolate, and optionally mannitol. Last, sodium stearyl fumarate was added to the composition, and the composition was blended for two minutes. These formulations did not show the improvement in dissolution. Without wishing to be bound by any one theory, the inventors hypothesize that powdered cannabinoids cannot be introduced into colloidal silicon dioxide without first being heated to a liquid state.

Formulation E[F] was produced according to the following process. CBD isolate and colloidal silicon dioxide particles were grinded together with a mortar and pestle. The composition containing CBD isolate and colloidal silicon dioxide was blended with silicified microcrystalline cellulose and sodium starch glycolate. Last, sodium stearyl fumarate was added to the composition, and the composition was blended.

### Methods of Making Tablets

Each of the solid cannabinoid powders described above was compressed in a GlobePharma Mini Press-II tablet press. This press has ten stations of tooling with five stations of B tooling and five stations of D tooling. The tablet diameter of the tooling for each tablet is provided in Table 3D.

TABLE 3A

| | | | K (% | L (% | M (% | M[H] (% |
|---|---|---|---|---|---|---|
| | I (% w/w) | J (% w/w) | w/w) | w/w) | w/w) | w/w) |
| Non-crystalline | 41.9 | 22.1 | 22.1 | 22.1 | 22.1 | 19.4 |
| cannabinoid oil | | | | | | |
| Colloidal silicon | 41.9 | 58.2 | 58.2 | 29.1 | 29.1 | 31.1 |
| dioxide | | | | | | |
| Silicified | | | | | | 46.6 |
| Microcrystalline | | | | | | |
| cellulose | | | | | | |
| Mannitol | 12.3 | 14.3 | 14.3 | 40.7 | 43.4 | |
| Sodium starch | 2.0 | 2.7 | 2.7 | 5.5 | 2.7 | |
| glycolate | | | | | | |
| Croscarmellose | | | | | | 1.9 |
| sodium | | | | | | |
| Sodium stearyl | 2.0 | 2.7 | 2.7 | 2.7 | 2.7 | 1.0 |
| fumarate | | | | | | |

TABLE 3B

| | L[H] (% | K[H] | J[H] (% | I[H] (% | H[H] | G[H] |
|---|---|---|---|---|---|---|
| | w/w) | (% w/w) | w/w) | w/w) | (% w/w) | (% w/w) |
| Non-crystalline | 19.4 | 19.4 | 19.4 | 16.2 | 19 | 19.4 |
| cannabinoid oil | | | | | | |
| Colloidal silicon | 31.1 | 15.5 | | | | |
| dioxide | | | | | | |
| Silicified | 46.6 | 62.1 | 77.7 | 81.0 | 76.2 | 77.7 |
| Microcrystalline | | | | | | |
| cellulose | | | | | | |
| Mannitol | | | | | | |
| Sodium starch | | | | | | |
| glycolate | | | | | | |
| Croscarmellose | 1.9 | 1.9 | 1.9 | 1.9 | 3.8 | 1.9 |
| sodium | | | | | | |
| Sodium stearyl | 1 | 1 | 1 | 0.8 | 1 | 1 |
| fumarate | | | | | | |

TABLE 3C

| | | E[H] | D[H] | | B[H] | A[H] |
|---|---|---|---|---|---|---|
| | F[H] (% | (% | (% | C[H] (% | (% | (% |
| | w/w) | w/w) | w/w) | w/w) | w/w) | w/w) |
| CBD Isolate | 25 | 10 | 6.7 | 25 | 25 | 26.5 |
| CBG Isolate | 9.8 | | 2.6 | 9.7 | 9.8 | 10.3 |
| Non-crystalline | 2.5 | | 0.7 | 2.5 | 2.5 | 2.7 |
| cannabinoid oil | | | | | | |
| Colloidal silicon | 37.5 | 17.5 | 50 | 37.5 | 37.5 | 37.1 |
| dioxide | | | | | | |
| Silicified | 21.5 | 68.8 | 37.3 | 22.5 | 22.5 | 18.6 |
| Microcrystalline | | | | | | |
| cellulose | | | | | | |
| Mannitol | | | | | | 1.9 |
| Sodium starch | 3 | 3 | 2 | 2 | 2 | 2 |
| glycolate | | | | | | |
| Croscarmellose | | | | | | |
| sodium | | | | | | |
| Sodium stearyl | 0.8 | 0.8 | 0.8 | 0.8 | 0.8 | 1 |
| fumarate | | | | | | |

TABLE 3D

| | Tablet Diameter of Tooling | | Tablet Tooling Shape |
|---|---|---|---|
| I | 13 | mm | Round |
| J | 7 | mm | Round |
| K | 7 | mm | Round |
| L | 7 | mm | Round |
| M | 7 | mm | Round |
| M[H] | 0.25 inches (in.) | | Round, Flat Face Bevel Edge |
| L[H] | 0.25 | in. | Round, Flat Face Bevel Edge |
| K[H] | 0.25 | in. | Round, Flat Face Bevel Edge |
| J[H] | 0.25 | in. | Round, Flat Face Bevel Edge |
| I[H] | 0.25 | in. | Round, Flat Face Bevel Edge |
| H[H] | 0.25 | in. | Round, Flat Face Bevel Edge |
| G[H] | 0.25 | in. | Round, Flat Face Bevel Edge |
| D[H] | 0.3125 | in. | Round, Standard Convex |
| C[H] | 0.25 | in. | Round, Flat Face Bevel Edge |
| B[H] | 0.3125 | in. | Round, Standard Convex |
| A[H] | 0.3125 | in. | Round, Standard Convex |

### Example 4—Tablet Parameters

Having prepared various tablets containing cannabinoid oil (see Example 3), the tablets were evaluated for their suitability as pharmaceutical products.

### Methods for Determining Hardness or Compression Force of Tablet

The hardness was determined using a “breaking force” test. Briefly, tablets were placed in a SOTAX HT1 Tablet Hardness Tester (FIG. 4). A force was applied to the tablet to cause fracture. The force at which the tablet fractures is the tablet's hardness.

The compression force of the tablet was determined by measuring the force acting on the tablet when the tablet was pressed in the tablet press.

### Methods for Determining Disintegration of Tablet

Each tablet was placed into a receptacle containing 10 milliliters (mL) of 2% (w/v) sodium lauryl sulfate (SLS). The amount of time that the tablet took to disintegrate was recorded. The tablet was determined to be disintegrated when no palpably firm core was remaining in the SLS.

### Methods for Determining Dissolution of Tablet

Tablets were analyzed for their dissolution performance using a Sotax 6-vessel Dissolution Bath (a USP Type II Dissolution Apparatus) in tandem with the Pion μDiss Profiler, which is a fiber optic dissolution unit. Each vessel is filled with 900 milliliters (mL) of 1% w/v Tween 80 (Polysorbate 80) solution. The vessel is heated to 37° C., and the stir speed is set to 100 revolutions per minute (RPM). Paddles were used for stirring. Each vessel had a single Pion probe submerged in the media to read the absorbance, which was translated to Percent (%) Dissolved.

Table 4A summarizes the parameters of each tablet. The data showed that tablets comprising formulations D[H], C[H], B[H], and A[H] were not hard enough. The hardness of these tablets was less than 50 Newtons (N). Tablets comprising formulations G[H], H[H], I[H], J[H], K[H], and L[H] each had suitable hardness, but the dissolution was not suitable (dissolution in 60 minutes<65%). Tablets comprising formulations M[H], M, L, K, J, and I each resulted in tablets of suitable hardness (50-100 N) and disintegration (1-10 minutes in 2% SLS).

This data showed that the excipient combination affected the suitability of the tablets for pharmaceutical formulation. This data also showed that the method of preparing the solid cannabinoid powder affects the suitability of a tablet as a pharmaceutical. Specifically, initial high shear mixing of non-crystalline cannabinoid oil and the colloidal silicon dioxide particles in the absence of other excipients is critical. The powder of tablet M[H] is prepared using this critical initial step and has the appropriate hardness and dissolution for a pharmaceutical. In contrast, the powder of tablet L[H] is produced by initial high shear mixing of colloidal silicon dioxide particles and non-crystalline cannabinoid oil with another excipient (i.e., silicified microcrystalline cellulose). In contrast to tablet M[H], Tablet L[H] was not a suitable pharmaceutical because it did not have the appropriate dissolution. The dissolution of Tablet M[H] was 77.5%, whereas the dissolution of Tablet L[H] was 57.3%.

TABLE 4A

| | | | Average | |
|---|---|---|---|---|
| | | Hardness | Compression | |
| | Disintegration | (Newtons (N)) | Force | Dissolution |
| I | N.D. | N.D. | 10 | N.D. |
| J | 1 minute in 2% | N.D. | 12.7 | N.D. |
| | sodium lauryl | | | |
| | sulfate (SLS) | | | |
| K | 1 minute in 2% | N.D. | 10 | N.D. |
| | SLS | | | |
| L | 1 minute in 2% | N.D. | N.D. | N.D. |
| | SLS | | | |
| M | 1 minute in 2% | N.D. | N.D. | N.D. |
| | SLS | | | |
| M[H] | N.D. | 73 | N.D. | 77.5% |
| L[H] | N.D. | 83 | N.D. | 57.3% |
| K[H] | N.D. | 70 | N.D. | 56.0% |
| J[H] | N.D. | 90 | N.D. | 61.4% |
| I[H] | N.D. | 90 | N.D. | 58.4% |
| H[H] | N.D. | | N.D. | 64.9% |
| G[H] | N.D. | | N.D. | 60.1% |
| F[H] | N.D. | N.D. | N.D. | N.D. |
| E[H]* | N.D. | | N.D. | 63.4% |
| D[H] | N.D. | <50 N | N.D. | 75.2% |
| C[H] | N.D. | <50 N | N.D. | 63.4% |
| B[H] | N.D. | 17 | N.D. | 49.7% |
| A[H] | N.D. | 10-20 | N.D. | 33.7% (30 min) |

*Powder blend

### Example 5—Capsules Containing Non-Crystalline Cannabinoid Oil

Examples 1 and 2 provide one example of the stabilization and dissolution benefits of colloidal silicon dioxide and cannabinoid combination for capsules. This example describes the development and formulation of additional capsules.

Specifically, it was discovered that additional ingredients could improve the commercial properties of cannabinoid-based capsules. Assessment of each of the tested formulations is described in more detail below and in Example 6.

Capsules comprising combinations of a non-crystalline cannabinoid oil, colloidal silicon dioxide, a filler (i.e., microcrystalline cellulose), a disintegrant (i.e. sodium starch glycolate), a lubricant (i.e. sodium stearyl fumarate), and flavorings (i.e., myrcene or linalool) were prepared using the methods described below. The components of each composition evaluated are contained in Tables 5A and 5B. Each table contains the percent by weight of each component of the compositions.

### Methods of Making Solid Cannabinoid Powders

Formulations G[C], F[C], E[C], and D[C]-Powders that Formed Suitable Commercial Grade Capsules

Formulation D[C] was generated according to the following process. The non-crystalline cannabinoid oil was added to colloidal silicon dioxide particles and combined vigorously with a mortar and pestle. The composition containing colloidal silicon dioxide particles and non-crystalline cannabinoid oil was transferred to a stainless steel mixing bowl. The cannabinoid isolates, silicified microcrystalline cellulose, and lubricant were added to the bowl, and mixed using a stand mixer at the lowest speed setting for five minutes. 69 grams of the formulation was used to fill 300 size 0 capsule shells.

Formulation E-C was generated according to the following process. The non-crystalline cannabinoid oil and terpenes were combined with colloidal silicon dioxide particles and grinded and pulsed until uniform. The mixture of colloidal silicon dioxide particles and non-crystalline cannabinoid oil were transferred to a stainless steel mixing bowl. CBD and CBG isolate was added to the bowl, and the mixture was mixed at the lowest speed for five minutes with a flat beater attachment. Sodium stearyl fumarate was added to the bowl, and the composition was mixed for 1 minute. The composition was used to fill size 2 capsule shells.

Formulations G-C and F-C were generated according to the following process. The non-crystalline cannabinoid oil and terpenes were combined with colloidal silicon dioxide and grinded and pulsed ten times. The mixture of colloidal silicon dioxide and non-crystalline cannabinoid oil were transferred to a stainless steel mixing bowl. CBD and CBG isolate was added to the bowl, and the mixture was mixed at the lowest speed for ten minutes with a flat beater attachment. Sodium stearyl fumarate was added to the bowl, and the composition was mixed for 2 minute. The composition was used to fill size 2 capsule shells.

Formulations C[C], B[C], and A[C]-Powders that Did not Form Suitable Capsules

Formulations C[C], B[C], and A[C] were generated according to the following process. CBD, CBG, and non-crystalline cannabinoid oil were combined with colloidal silicon dioxide particles and grinded together in a mortar and pestle. Silicified microcrystalline cellulose, sodium starch glycolate were added to the mixture, and it was blended. Sodium stearyl fumarate was added last, and the mixture was blended.

### Capsule Preparation

The solid cannabinoid powders above were filled in gelatin or vegetarian shells. A Torpac ProFiller® 3700 capsule filler was used to fill the capsule shells with Cannabinoid Products. 230 mg of the D[C] Cannabinoid Product was added to size 0 gelatin capsules. 123.8 mg of the G[C], F[C], or E[C] Cannabinoid Products were added to size 2 gelatin capsules. FIG. 2 shows an image of the Torpac ProFiller® 3700 capsule filler. The vegetarian shells contained hydroxypropylmethylcellulose.

TABLE 5A

| | G[C] (% | F[C] (% | E[C] (% | D[C] (% |
|---|---|---|---|---|
| | w/w) | w/w) | w/w) | w/w) |
| Cannabidiol (CBD) | 40.4 | 40.4 | 40.4 | 21.7 |
| Isolate | | | | |
| Cannabigerol (CBG) | 11.7 | 11.7 | 11.7 | 8.5 |
| Isolate | | | | |
| Non-crystalline | | | | 2.2 |
| cannabinoid oil | | | | |
| (broad spectrum) | | | | |
| Non-crystalline | 8.1 | 8.1 | 8.1 | |
| cannabinoid oil (D8) | | | | |
| Colloidal silicon | 39 | 39 | 39 | 32.4 |
| dioxide | | | | |
| Silicified | | | | 34.8 |
| Microcrystalline | | | | |
| cellulose | | | | |
| Sodium stearyl | 0.4 | 0.4 | 0.4 | 0.4 |
| fumarate | | | | |
| Myrcene | 0.2 | 0.2 | 0.2 | |
| Linalool | 0.2 | 0.2 | 0.2 | |

TABLE 5B

| | C[C] (% | B[C] (% | A[C] (% |
|---|---|---|---|
| | w/w) | w/w) | w/w) |
| Cannabidiol (CBD) Isolate | 6.7 | 6.7 | 6.7 |
| (not loaded) | | | |
| Cannabigerol (CBG) | 2.6 | 2.6 | 2.6 |
| Isolate | | | |
| (not loaded) | | | |
| Non-crystalline | 0.7 | 0.7 | 0.7 |
| cannabinoid oil (broad | | | |
| spectrum) | | | |
| Non-crystalline | | | |
| cannabinoid oil (D8) | | | |
| Colloidal silicon dioxide | 17.5 | 15 | 12.5 |
| Silicified Microcrystalline | 68.8 | 71.3 | 73.8 |
| cellulose | | | |
| Sodium starch glycolate | 3 | 3 | 3 |
| Sodium stearyl fumarate | 0.8 | 0.8 | 0.8 |

### Example 6—Capsule Parameters

Having prepared various capsules containing non-crystalline cannabinoid oil (see Example 5), the capsules were evaluated for their suitability as pharmaceutical products.

### Methods for Determining Dissolution of Capsule

Dissolution measurements were performed in a Sotax 6-vessel Dissolution Bath (a USP Type 1 Dissolution Apparatus) in tandem with the Pion μDiss Profiler, which is a fiber optic dissolution unit. Each vessel was filled with 900 milliliters (mL) of i) fasted state simulated intestinal fluid (FaSSIF), or ii) a fed state simulated intestinal fluid (FeSSIF). The FaSSIF contained about 3 mM taurocholate, 0.75 mM phospholipids, 148 mM sodium ions, 106 mM chloride, and 29 mM phosphate ions, thereby modeling the conditions present in fasted small intestine gastrointestinal fluid. The pH of FaSSIF was about 6.5. FeSSIF contained about 15 mM taurocholate, 3.75 mM phospholipids, 319 mM sodium ions, 203 mM chloride, and 144 mM acetic acid. The pH of FeSSIF was about 5.0. FeSSIF contained similar osmolality and pH to fed intestinal fluid. A iii) control polysorbate 80 (TWEEN® 80) solution was also tested in the experiment.

The vessel was heated to 37° C., and the stir speed was set to 100 revolutions per minute (RPM). Each vessel had a single Pion probe submerged in the media to read the absorbance, which was translated to Percent (%) Dissolved. Baskets were used for stirring the capsules.

Table 6A summarizes the results from this experiment as percent dissolution within 60 minutes (higher value indicates greater dissolution).

### Results

Table 6A provides the dissolution data for each capsule.

TABLE 6A

| Formulations | Control | FaSSIF | FeSSIF |
|---|---|---|---|
| G[C] | N.D. | N.D. | N.D. |
| F[C] | 27.9% | 12.0% | 37.2% |
| E[C] | 45.4% | 8.4% | 38.6% |
| D[C] | 37.1% | 18.9% | 41.7% |
| C[C]* | 83.6% | 27.7% | N.D. |
| B[C]* | 60.1% | 23.7% | N.D. |
| A[C]* | 70.0% | 25.8% | N.D. |

*Powder blend, not filled into capsule

### Example 7: Formulation of ZLT-L-007

Diabetic neuropathy affects greater than 200,000 Americans. Pain killers, such as opioids, are often prescribed to a patient to help cope with pain; however, opioids are addictive substances that can have devastating side-effects.

To overcome these challenges, a novel product was developed to treat pain associated with diabetic neuropathy, ZLT-L-007, that uses the tablets and capsules described in Examples 1-6. ZLT-L-007 contains a natural spectrum of cannabinoid and non-naturally occurring ratios of terpenes that can be sourced from the cannabis plant, or other chemical or biosynthetic means.

ZLT-L-007 comprises CBD, CBG, Δ8 THC, and individual isolates of terpenes.

The specific weights of each component of ZLT-L-007 are provided in Tables 7 & 8.

TABLE 7
Cannabinoids and Terpenes of ZLT-L-007
ZLT-L-007 Formula
Cannabinoids

| | | |
|---|---|---|
| Cannabidiol | 50 | mg |
| Δ8-THC | 10 | mg |
| Cannabigerol | 19.50 | mg |

| | |
|---|---|
| Total Cannabinoid | 75 mg/capsule |

Terpenes

| | | | |
|---|---|---|---|
| Myrcene Terpene | 0.25 | mg | |
| Linalool Terpene | 0.25 | mg | 0.50 mg/capsule |

| | |
|---|---|
| Other non-active | Colloidal Silicon Dioxide, Sodium |
| excipients include: | Starch Glycolate, Sodium Stearyl |
| | Fumarate and Mannitol |

TABLE 8
Percentages and Weights for ZLT-L-007 Components

| | | | Theoretical |
|---|---|---|---|
| Component | % w/w | mg/capsule | Batch Size (g)¹ |
| Cannabidiol (CBD) | 40.4% | 50.0 | 750.0 |
| Cannabigerol (CBG) | 11.7% | 14.50 | 217.5 |
| Δ8-THC | 8.1% | 10.0 | 150.0 |
| Colloidal silicon dioxide | 39.0% | 48.30 | 724.5 |
| Sodium Stearyl Fumarate | 0.4% | 0.05 | 7.5 |
| Myrcene terpene | 0.2% | 0.2 | 3.8 |
| Linalool terpene | 0.2% | 0.2 | 3.8 |
| Capsule, Size 2 Gelatin | — | 60.9 | — |
| Total | 100.0% | 184.7 | 1857.0 |

### Example 8: Clinical Study to Evaluate ZLT-L-007 in Subjects with Pain Associated with Diabetic Neuropathy

To determine the effect of ZLT-L-007 in subjects with pain associated with diabetic neuropathy, an observational 12-week clinical study to evaluate the efficacy, safety and tolerability of ZLT-L-007 versus Pregabalin (300 Mg/Day) was conducted.

### Primary Endpoints

Primary endpoints of the study included: 1. Recording Daily Pain Numeric Rating Scale (NRS) at Baseline, Day 30, Day 60, and Day 90.

### Secondary Endpoints

Secondary endpoints of the study included:
1. Recording Neuropathic Pain Symptom Inventory (NPSI)—Burning (Superficial) Spontaneous Pain at Baseline, Day 30, Day 60 and Day 90.
2. Recording a change in Short Form McGill Pain Questionnaire (SF-MPQ) Score at Day 30, Day 60 and Day 90. SF-MPQ was assessed according to the participant's answer to the SF-MPQ questionnaire. The score for each composite scale (sensory, affective, and total) was derived by summing the reported intensity value for each item within a particular scale where None=0, Mild=1, Moderate=2, and Severe=3. The sensory score was the sum of the scores of the first 11 pain descriptors (throbbing, shooting, stabbing, sharp, cramping, gnawing, hot-burning, aching, heavy, tender, and splitting) and could range from 0-33. The affective score was the sum of the scores of the last 4 pain descriptors (tiring-exhausting, sickening, fearful, and punishing-cruel) and could range from 0-12. The total score was the sum of the scores of all 15 pain descriptors and could range from 0 to 45. Higher scores indicated greater pain.
3. Recording Baseline Pain Visual Analogue Scale (VAS). The VAS was part of the Short Form McGill Pain Questionnaire (SF-MPQ) scale and reflected the overall pain intensity score, the pain VAS was a horizontal line; 100 millimeters (mm) in length, was self-administered by the participant in order to rate pain from 0 (no pain) to 100 (worst possible pain). The PPI was part of the SF-MPQ scale and measured the participant's present pain intensity on a 6-point scale ranging from 0 (no pain) to 5 (excruciating) at Baseline, Day 30, Day 60 and Day 90.
4. Recording Daily Sleep Interference Scale Score (DSIS) at Baseline, Day 30, Day 60 and Day 90.
5. Recording Clinical Global Impression of Change (CGIC) at Baseline, Day 30, Day 60, and Day 90. The CGIC was a clinician-rated global measure that provided a clinically relevant and easy to interpret account of a clinician's perception of the clinical importance of the participant's improvement or worsening during their involvement in a clinical study. Clinicians rated the participant's overall improvement on a 7-point scale where scores ranged from 1 (very much improved) to 7 (very much worse).
6. Recording Patient Global Impression of Change (PGIC) Score at Baseline, Day 30, Day 60 and Day 90. The PGIC was a participant-rated global measure that provided a clinically relevant and easy to interpret account of a participant's perception of the clinical importance of their own improvement or worsening during their involvement in a clinical study. Participants rated their overall improvement on a 7-point scale where scores ranged from 1 (very much improved) to 7 (very much worse).
7. Recording Baseline Hospital Anxiety and Depression Scale (HADS) Scores—The HADS was a self-administered questionnaire that consisted of 2 subscales, 1 measuring anxiety (HADS-A Scale) and the other measuring depression (HADS-D Scale). Each subscale comprised 7 items; participants assessed how each item applied to them on a scale of 0 (no presence of anxiety or depression) to 3 (severe feeling of anxiety or depression). Subscores from HADS-A (Anxiety) and HADS-D (Depression) were not combined. The interpretation of each HADS subscales will be as follows: 0-7 normal, 8-10 mild, 11-14 moderate and 15-21 severe at Baseline, Day 30, Day 60 and Day 90.
8. Recording weight gain captured in the vitals at Day 30, Day 60 and Day 90.
9. Recording a change in daily total activity Counts Per Day per Subject Questionnaire (measured steps and daytime activity) at Day 30, Day 60 and Day 90.
10. Recording a change in the total amount of rescue medication daily per Subject Questionnaire at Day 30, Day 60 and Day 90. Subjects continued with their standard of care rescue medications previously prescribed by their treating physician.
11. Recording a change in the daily loss of balance captured by the Subject Questionnaire at Day 30, Day 60 and Day 90.
12. Recording a change in the daily capture of sensory issues pins and needles, uncomfortable tingling and burning, oversensitivity, reduced sensation of touch, or sensitivity to pain captured by the Subject Questionnaire at Day 30, Day 60 and Day 90.
13. Recording a change in the daily capture of muscular issues, cramping, muscle weakness or problems with coordination captured per the Subject Questionnaire at Day 30, Day 60 and Day 90.
14. Recording the number of Participants with Treatment-Emergent Adverse Events (TEAEs), Serious Adverse Events (SAEs), and Withdrawals Due to Adverse Events (AEs) at Baseline, Day 30, Day 60 and Day 90. An AE was any untoward medical occurrence attributed to study drug in a participant who received study drug. An SAE was an AE resulting in any of the following outcomes or deemed significant for any other reason: death; life threatening (immediate risk of death); initial or prolonged inpatient hospitalization; persistent or significant disability/incapacity; resulted in congenital anomaly/birth defect.
15. Recording a change in blood pressure as captured in the vitals.

### Data

Data Capture was deployed to capture subject information in real-time. Adverse events were evaluated throughout the study. All serious adverse events (SAEs) were reported to the Sponsor or its designee within 24 hours of the investigator's learning of the event. Subjects as well as central labs were drawn as well as capturing vitals.

### Number of Subjects (Planned)

Approximately 60 subjects, all Pennsylvania Medical Marijuana Program participants, were included.

### Subject Inclusion Criteria

An individual was eligible for inclusion in this study if all of the following applied:
1. Males and Females between 18 years to 85 years.
2. In good general health, as determined by the Investigator.
3. Willing and able to attend all study visits and utilize ePro.
4. At the baseline and randomization visits, a score of >50 mm on the Visual Analogue Scale, at randomization, subjects must have completed at least 5 daily pain interference diaries, and have an average daily pain score of >5 over the past 7 days that is not currently being treated.
5. Patient who are willing and capable to comply with scheduled visits, treatment plan, laboratory tests, and other study procedures.
6. Women of childbearing potential are willing to use contraception during study.
7. Must be a Pennsylvania Medical Marijuana Cardholder (PA Resident).

An individual was not eligible for inclusion if:
1. The individual had current substance (non-prescription) or alcohol dependence.
2. The individual had dementia or Alzheimer's disease.
3. The individual had previous serious adverse event or hypersensitivity to medical cannabis or cannabinoids.
4. The individual had psychosis.
5. The individual had uncontrolled hypertension, defined as a systolic blood pressure greater than 160 mm Hg or a diastolic blood pressure greater than 100 mm Hg.
6. The individual is pregnant, breastfeeding, or unwilling to prevent pregnancy during the drug and cannabis administration portion of the study (using birth control in female participants of child-bearing age). If a subject became pregnant while in the study, they were withdrawn from the study and the Investigator followed the patient thereafter.
7. The individual had routine use of opioids greater than 60 mg; if none at all use of opioids.
8. The individual had concurrent cannabis or cannabis product use while participating in the study
9. The individual had greater than 30% decrease on the Pain Visual Analog Scale at randomization as compared to screening; and during the 1 week screening period, with more than one pain score<3 in pain scores.
10. The individual had other kinds of neurological disorders, pain of other reason, or a skin condition that confused the assessment.
11. The individual had any other serious or unstable condition which in the opinion of the investigator compromised participation in the study.

### Investigational Product, Dosage and Mode of Administration

Three groups were used to evaluate the effect of ZLT-L-007 in subjects with pain associated with diabetic neuropathy: Group 1 (Pregabalin), Group 2 (ZLT-L-007 pharmaceutical composition+terpene composition), and Group 3 (Pregabalin and ZLT-L-007 pharmaceutical composition+terpene composition). More detailed information regarding each group's treatment is provided below.

Group 1 Pregabalin (n=22):

Subjects in the pregabalin group were already taking pregabalin at the prescribed dose as recommended by their doctor.

Group 2 Pregabalin and ZLT-L-007 (n=20):

Group 3 subjects were already taking pregabalin/Lyrica® at the prescribed dose recommended by their doctor. Subjects then received one capsule by mouth twice daily of the ZLT-L-007 pharmaceutical composition. Participants were also given one capsule by mouth twice daily of a terpene composition that comprised 0.25 mg of myrcene and 0.25 mg of linalool.

If there was no response after two weeks, the Principal Investigator could increase the dosing of the ZLT-L-007 pharmaceutical composition and/or the terpene composition to three times daily. If the Principal Investigator felt that a subject needed an additional dose, an additional capsule was provided so that the subjects received a dose four times daily. Subjects will already be taking pregabalin at the prescribed dose as recommended by their doctor. Study drugs were taken by the subjects in the privacy of their own homes.

Group 3 ZLT-L-700 only (n=18):

Group 3 was administered one capsule by mouth twice daily of the ZLT-L-007 pharmaceutical composition.. Participants were also given one capsule by mouth twice daily of a terpene composition that comprised 0.25 mg of myrcene and 0.25 mg of linalool.

As with Group 2, if there was no response after two weeks, the Principal Investigator could increase the dosing of the ZLT-L-007 pharmaceutical composition and/or the terpene composition to three times daily.

If the Principal Investigator felt that a subject needed an additional dose, an additional capsule was provided so that the subject received a dose four times daily. Study drugs were taken by the subjects in the privacy of their own homes.

### Safety Measures

Several safety measures, including recording incidence of adverse events, vital signs, and central labs were in place throughout the study.

### Schedule of Assessments

The study comprised an initial visit, followed by an additional visit every 30 days. Detailed information regarding each visit is explained in Table 9.

TABLE 9
Schedule of Events

| | Study Day | | | |
|---|---|---|---|---|
| | | Day 30 | Day 60 | Day 90 |
| | | (+/−5 | (+/−5 | (+/−5 |
| | Day −0 | days) | days) | days) |
| | Study Visit # | | | |
| | Screening/ | | | |
| | Visit 1 | Visit 2 | Visit 3 | Visit 4 |
| Informed Consent | X | | | |
| Safety Evaluation | X | X | X | X |
| Adverse Event | | X | X | X |
| Medical History | X | | | |
| Vitals | X | X | X | X |
| Urine Pregnancy Test | X | | | |
| Inclusion/Exclusion | X | | | |
| Central Labs | X | X | X | X |
| Set Patient Up with ePro | X | X | X | X |
| Physical Exam | X | | | |
| Study Drug Accountability | X | X | X | X |
| Daily Pain Numeric Rating | X | X | X | X |
| Scale (NRS) | | | | |
| Neuropathic Pain Symptom | X | X | X | X |
| Inventory (NPSI) | | | | |
| DSIS | X | X | X | X |
| Treatment-Emergent Adverse | | X | X | X |
| Events (TEAEs) Tracking | | | | |
| McGill Pain | X | X | X | X |
| VAS | X | X | X | X |
| SF-MPQ | X | X | X | X |
| CGIC | X | X | X | X |
| DPRS | X | X | X | X |
| PGIC | X | X | X | X |
| Subject Questionnaire | X | X | X | X |
| HADS | X | X | X | X |

### Results

ZLT-L-007 achieved a significant reduction in NRS severity scores, surpassing Lyrica® and indicating a decrease in symptom severity over time. ZLT-L-007 was also found to be safe and well-tolerated, meeting the primary endpoint for safety with no Serious Adverse Events (SAE) reported.

The demographic and baseline characteristics of the trial are as follows. The mean age was 59.6 years for the Lyrica® (Pregabalin) only, 56.5 years for the ZLT-L-007 only, 61.3 years for the Lyrica® (Pregabalin)+ZLT-L-007 only, and 59.3 years for all participants. The median age ranged from 57.0 to 62.0 years, and the minimum and maximum ages varied from 33 to 86 years across the groups. Regarding gender, most participants were female in the Lyrica® (Pregabalin) only (Group 1) and Lyrica® (Pregabalin)+ZLT-L-007 (Group 2), while it was evenly split between male and female participants in the ZLT-L-007 only (Group 3). In terms of race, most participants were White, followed by Black and Multiracial individuals. All participants identified as not Hispanic or Latino.

The study met its primary endpoint for the change in daily pain severity measured by the % change from baseline at, Day 30, Day 60, and Day 90, on the Numerical Rating Scale (NRS).

For the NRS Severity parameter, the median values of the percent change from baseline for each treatment group is set forth below. This data provides valuable information on the magnitude of improvement within each treatment group:

### Lyrica®/(Pregabalin) Only (Group 1):

30-day follow-up: Median percent change from baseline: −33.33%
60-day follow-up: Median percent change from baseline: −20.00%
90-day follow-up: Median percent change from baseline: −35.00%

### ZLT-L-007 Only (Group 3):

30-day follow-up: Median percent change from baseline: −33.33%
60-day follow-up: Median percent change from baseline: −71.43%
90-day follow-up: Median percent change from baseline: −78.57%

### Lyrica®/Pregabalin+ZLT-L-007 (Group 2):

30-day follow-up: Median percent change from baseline: −20.00%
60-day follow-up: Median percent change from baseline: −50.00%
90-day follow-up: Median percent change from baseline: −72.50%

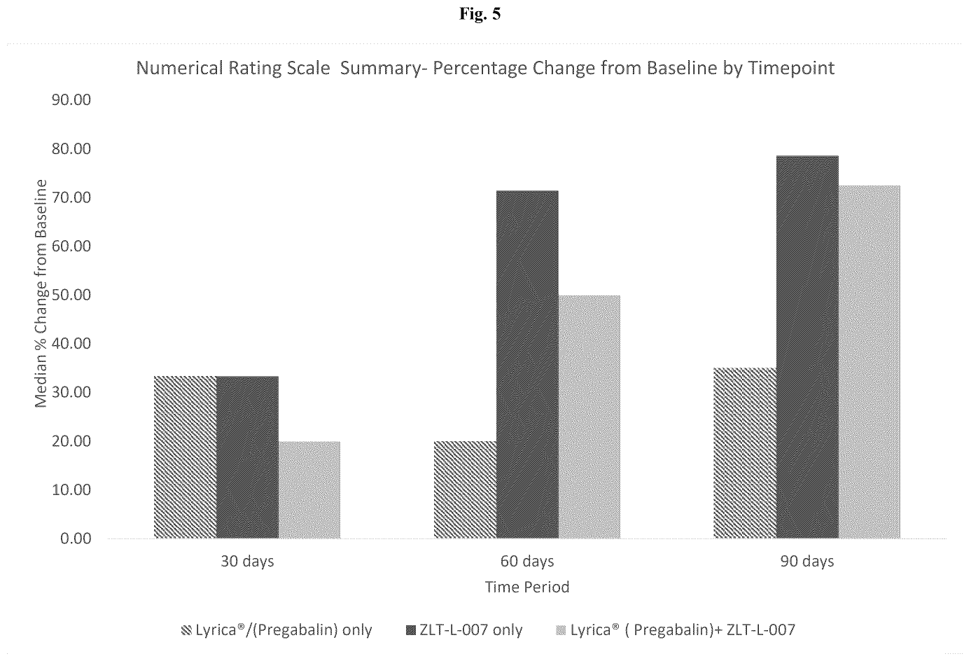

As shown in FIG. 5, for the reference Lyrica® (pregabalin) group, the median percent change from baseline suggests a reduction in symptom severity at all follow-up periods, ranging from −20.00% to −35.00%. For the ZLT-L-007 only group, the median percent change from baseline demonstrates a notable reduction in symptom severity at the 30-day follow-up (−33.33%). However, the median percent change from baseline at the 60-day and 90-day follow-ups (−71.43% and −78.57%, respectively) suggests a larger improvement in symptom severity than with Lyrica®/Pregabalin alone. The median percent change from baseline from the Lyrica® (Pregabalin)+ZLT-L-007 group shows a moderate reduction in symptom severity at the 30-day follow-up (−20.00%). However, the median percent change from baseline at the 60-day and 90-day follow-ups (−50.00% and −72.50%, respectively) indicates a substantial improvement in symptom severity than with Lyrica®/Pregabalin alone.

The study met another of its primary endpoint for safety measured by Serious Adverse Event (SAE) report with 0 SAE reports for the duration of the study.

The study met its secondary endpoint for the change in daily pain severity measured by the % change from baseline, Day 30, Day 60, Day 90 for Visual Analog Scale (VAS). The Lyrica®/Pregabalin only group demonstrated a decrease in pain severity from baseline at all three timepoints, with median percent changes ranging from −33.33% to −35.00%. This suggests that Lyrica®/Pregabalin alone effectively reduced pain levels in the study participants. The ZLT-L-007 only group exhibited a more substantial decrease in pain severity compared to the Lyrica®/Pregabalin only group at the 60-day and 90-day follow-ups. The median percent change from baseline ranged from −71.43% to −78.57%, indicating a potentially stronger analgesic effect of ZLT-L-007 in managing pain. The combination of Lyrica®/Pregabalin and ZLT-L-007 in the Lyrica®/Pregabalin+ZLT-L-007 group showed intermediate results, with median percent changes from baseline ranging from −20.00% to −72.50%. This suggests a potential synergistic effect of the combined treatment, but with less efficacy than ZLT-L-007 alone.

The study also met several secondary endpoints such as measurable change in the Short Form McGill Pain Questionnaire and NPSI. There was no significant change in systolic and diastolic blood pressure measures for the participants in the study measured at the different time points.

### NUMBERED EMBODIMENTS

Embodiment 1. A pharmaceutical composition for treating pain in a subject with diabetic neuropathy, the pharmaceutical composition comprising:
(a) cannabinoids;
(b) colloidal silicon dioxide;
(c) sodium stearyl fumarate; and,
(d) terpene component.

Embodiment 2. The pharmaceutical composition of embodiment 1, wherein the cannabinoids comprise Cannabidiol (CBD), Cannabigerol (CBG), and Δ8-Tetrahydrocannabinol (THC).

Embodiment 3. The pharmaceutical composition of any one of embodiments 1-2, wherein the pharmaceutical composition comprises between 30-50% (w/w) CBD.

Embodiment 4. The pharmaceutical composition of any one of embodiments 1-3, wherein the pharmaceutical composition comprises between 35-45% (w/w) CBD.

Embodiment 5. The pharmaceutical composition of any one of embodiments 1-4, wherein the pharmaceutical composition comprises about 40.4% (w/w) CBD.

Embodiment 6. The pharmaceutical composition of any one of embodiments 1-5, wherein the pharmaceutical composition comprises between 1-20% (w/w) CBG.

Embodiment 7. The pharmaceutical composition of any one of embodiments 1-6, wherein the pharmaceutical composition comprises between 5-15% (w/w) CBG.

Embodiment 8. The pharmaceutical composition of any one of embodiments 1-7, wherein the pharmaceutical composition comprises between 10-12.5% (w/w) CBG.

Embodiment 9. The pharmaceutical composition of any one of embodiments 1-8, wherein the pharmaceutical composition comprises about 11.7% (w/w) CBG.

Embodiment 10. The pharmaceutical composition of any one of embodiments 1-9, wherein the pharmaceutical composition comprises between 1-20% (w/w) Δ8-THC.

Embodiment 11. The pharmaceutical composition of any one of embodiments 1-10, wherein the pharmaceutical composition comprises between 5-15% (w/w) Δ8-THC.

Embodiment 12. The pharmaceutical composition of any one of embodiments 1-11, wherein the pharmaceutical composition comprises between 7.5-15% (w/w) Δ8-THC.

Embodiment 13. The pharmaceutical composition of any one of embodiments 1-12, wherein the pharmaceutical composition comprises about 8.1% (w/w) Δ8-THC.

Embodiment 14. The pharmaceutical composition of any one of embodiments 1-13, wherein the pharmaceutical composition comprises between 0.01-2% (w/w) terpene component.

Embodiment 15. The pharmaceutical composition of any one of embodiments 1-14, wherein the pharmaceutical composition comprises between 0.1-1% (w/w) terpene component.

Embodiment 16. The pharmaceutical composition of any one of embodiments 1-15, wherein the pharmaceutical composition comprises about 0.4% (w/w) terpene component.

Embodiment 17. The pharmaceutical composition of any one of embodiments 1-16, wherein terpene component comprises at most one terpene.

Embodiment 18. The pharmaceutical composition of any one of embodiments 1-17, wherein terpene component comprises at least one terpene.

Embodiment 19. The pharmaceutical composition of any one of embodiments 1-18, wherein terpene component comprises at least two terpenes.

Embodiment 20. The pharmaceutical composition of any one of embodiments 1-19, wherein the terpene component comprises myrcene terpene.

Embodiment 21. The pharmaceutical composition of any one of embodiments 1-20, wherein the terpene component comprises linalool terpene.

Embodiment 22. The pharmaceutical composition of any one of embodiments 1-21, wherein the terpene component comprises myrcene terpene and linalool terpene.

Embodiment 23. The pharmaceutical composition of any one of embodiments 1-22, wherein the pharmaceutical composition comprises between 0.01-1% (w/w) myrcene terpene.

Embodiment 24. The pharmaceutical composition of any one of embodiments 1-23, wherein the pharmaceutical composition comprises between 0.1-0.5% (w/w) myrcene terpene.

Embodiment 25. The pharmaceutical composition of any one of embodiments 1-24, wherein the pharmaceutical composition comprises about 0.2% (w/w) myrcene terpene.

Embodiment 26. The pharmaceutical composition of any one of embodiments 1-25, wherein the pharmaceutical composition comprises between 0.01-1% (w/w) linalool terpene.

Embodiment 27. The pharmaceutical composition of any one of embodiments 1-26, wherein the pharmaceutical composition comprises between 0.1-0.5% (w/w) linalool terpene.

Embodiment 28. The pharmaceutical composition of any one of embodiments 1-27, wherein the pharmaceutical composition comprises about 0.2% (w/w) linalool terpene.

Embodiment 29. The pharmaceutical composition of any one of embodiments 1-28, wherein the pharmaceutical composition comprises between 0.01-1% (w/w) linalool terpene and/or 0.01-1% (w/w) myrcene terpene.

Embodiment 30. The pharmaceutical composition of any one of embodiments 1-29, wherein the pharmaceutical composition comprises between 0.1-0.5% (w/w) linalool terpene and/or 0.1-0.5% (w/w) myrcene terpene.

Embodiment 31. The pharmaceutical composition of any one of embodiments 1-30, wherein the pharmaceutical composition comprises about 0.2% (w/w) linalool terpene and/or about 0.2% (w/w) myrcene terpene.

Embodiment 32. The pharmaceutical composition of any one of embodiments 1-31, wherein the pharmaceutical composition comprises between 0.01-1% (w/w) sodium stearyl fumarate.

Embodiment 33. The pharmaceutical composition of any one of embodiments 1-32, wherein the pharmaceutical composition comprises between 0.1-0.6% (w/w) sodium stearyl fumarate.

Embodiment 34. The pharmaceutical composition of any one of embodiments 1-33, wherein the pharmaceutical composition comprises about 0.4% (w/w) sodium stearyl fumarate.

Embodiment 35. The pharmaceutical composition of any one of embodiments 1-34, wherein the pharmaceutical composition comprises between 30-50% (w/w) colloidal silicon dioxide.

Embodiment 36. The pharmaceutical composition of any one of embodiments 1-35, wherein the pharmaceutical composition comprises between 35-45% (w/w) colloidal silicon dioxide.

Embodiment 37. The pharmaceutical composition of any one of embodiments 1-36, wherein the pharmaceutical composition comprises about 39% (w/w) colloidal silicon dioxide.

Embodiment 38. The pharmaceutical composition of any one of embodiments 1-37, wherein the pharmaceutical composition is in the form of a tablet.

Embodiment 39. The pharmaceutical composition of any one of embodiments 1-38, wherein the pharmaceutical composition is in the form of a powder capsule.

Embodiment 40. A pharmaceutical composition that comprises:
(a) a cannabinoids;
(b) a terpene component; and,
(c) non-active excipients.

Embodiment 41. The pharmaceutical composition of embodiment 40, wherein the non-active excipients comprise colloidal Silicon Dioxide, Sodium Starch Glycolate, Sodium Stearyl Fumarate and/or Mannitol.

Embodiment 42. The pharmaceutical composition of any one of embodiments 40-41, wherein the pharmaceutical composition is administered to a subject in a capsule.

Embodiment 43. The pharmaceutical composition of any one of embodiments 40-42, wherein the pharmaceutical composition is administered to a subject in a tablet.

Embodiment 44. The pharmaceutical composition of any one of embodiments 40-43, wherein the cannabinoids comprise Cannabidiol (CBD), Cannabigerol (CBG), and Δ8-Tetrahydrocannabinol (THC).

Embodiment 45. The pharmaceutical composition of any one of embodiments 40-44, wherein the pharmaceutical composition comprises between 30-70 mg CBD.

Embodiment 46. The pharmaceutical composition of any one of embodiments 40-45, wherein the pharmaceutical composition comprises between 40-60 mg CBD.

Embodiment 47. The pharmaceutical composition of any one of embodiments 40-46, wherein the pharmaceutical composition comprises about 50 mg CBD.

Embodiment 48. The pharmaceutical composition of any one of embodiments 40-47, wherein the pharmaceutical composition comprises between 5-40 mg CBG.

Embodiment 49. The pharmaceutical composition of any one of embodiments 40-48, wherein the pharmaceutical composition comprises between 10-30 mg CBG.

Embodiment 50. The pharmaceutical composition of any one of embodiments 40-49, wherein the pharmaceutical composition comprises between 15-25 mg CBG.

Embodiment 51. The pharmaceutical composition of any one of embodiments 40-50, wherein the pharmaceutical composition comprises about 19.5 mg CBG.

Embodiment 52. The pharmaceutical composition of any one of embodiments 40-51, wherein the pharmaceutical composition comprises between 1-30 mg Δ8-THC.

Embodiment 53. The pharmaceutical composition of any one of embodiments 40-52, wherein the pharmaceutical composition comprises between 5-20 mg Δ8-THC.

Embodiment 54. The pharmaceutical composition of any one of embodiments 40-53, wherein the pharmaceutical composition comprises between 7.5-15 mg Δ8-THC.

Embodiment 55. The pharmaceutical composition of any one of embodiments 40-54, wherein the pharmaceutical composition comprises about 10 mg Δ8-THC.

Embodiment 56. The pharmaceutical composition of any one of embodiments 40-55, wherein the pharmaceutical composition comprises between 0.1-2 mg terpene component.

Embodiment 57. The pharmaceutical composition of any one of embodiments 40-56, wherein the pharmaceutical composition comprises between 0.25-1 mg terpene component.

Embodiment 58. The pharmaceutical composition of any one of embodiments 40-57, wherein the pharmaceutical composition comprises about 0.5 mg terpene component.

Embodiment 59. The pharmaceutical composition of any one of embodiments 40-58, wherein terpene component comprises at most one terpene.

Embodiment 60. The pharmaceutical composition of any one of embodiments 40-59, wherein terpene component comprises at least one terpene.

Embodiment 61. The pharmaceutical composition of any one of embodiments 40-60, wherein terpene component comprises at least two terpenes.

Embodiment 62. The pharmaceutical composition of any one of embodiments 40-61, wherein the terpene component comprises myrcene terpene.

Embodiment 63. The pharmaceutical composition of any one of embodiments 40-62, wherein the terpene component comprises linalool terpene.

Embodiment 64. The pharmaceutical composition of any one of embodiments 40-63, wherein the terpene component comprises myrcene terpene and linalool terpene.

Embodiment 65. The pharmaceutical composition of any one of embodiments 40-64, wherein the pharmaceutical composition comprises between 0.01-1 mg myrcene terpene.

Embodiment 66. The pharmaceutical composition of any one of embodiments 40-65, wherein the pharmaceutical composition comprises between 0.1-0.5 mg myrcene terpene.

Embodiment 67. The pharmaceutical composition of any one of embodiments 40-66, wherein the pharmaceutical composition comprises about 0.25 mg myrcene terpene.

Embodiment 68. The pharmaceutical composition of any one of embodiments 40-67, wherein the pharmaceutical composition comprises between 0.01-1 mg linalool terpene.

Embodiment 69. The pharmaceutical composition of any one of embodiments 40-68, wherein the pharmaceutical composition comprises between 0.1-0.5 mg linalool terpene.

Embodiment 70. The pharmaceutical composition of any one of embodiments 40-69, wherein the pharmaceutical composition comprises about 0.25 mg linalool terpene.

Embodiment 71. The pharmaceutical composition of any one of embodiments 40-70, wherein the pharmaceutical composition comprises between 0.01-1 mg linalool terpene and/or 0.01-1 mg myrcene terpene.

Embodiment 72. The pharmaceutical composition of any one of embodiments 40-71, wherein the pharmaceutical composition comprises between 0.1-0.5 mg linalool terpene and/or 0.1-0.5 mg myrcene terpene.

Embodiment 73. The pharmaceutical composition of any one of embodiments 40-72, wherein the pharmaceutical composition comprises about 0.25 mg linalool terpene and/or about 0.25 mg myrcene terpene.

Embodiment 74. The pharmaceutical composition of any one of embodiments 40-73, wherein the pharmaceutical composition is in the form of a tablet.

Embodiment 75. The pharmaceutical composition of any one of embodiments 40-74, wherein the pharmaceutical composition is in the form of a powder capsule.

Embodiment 76. A method of treating pain in a subject with diabetic neuropathy by administering to
the subject a dose at least twice daily, the dose comprising:
   (a) cannabinoids;
   (b) colloidal silicon dioxide;
   (c) sodium stearyl fumarate; and,
   (d) terpene component,
wherein, the subject's pain improves compared to a subject that is not administered the dose.

Embodiment 77. The method of embodiment 76, wherein the cannabinoids comprise Cannabidiol (CBD), Cannabigerol (CBG), and Δ8-Tetrahydrocannabinol (THC).

Embodiment 78. The method of any one of embodiments 76-77, wherein the dose comprises between 20-50% (w/w) CBD.

Embodiment 79. The method of any one of embodiments 76-78, wherein the dose comprises between 35-45% (w/w) CBD.

Embodiment 80. The method of any one of embodiments 76-79, wherein the dose comprises about 40.4% (w/w) CBD.

Embodiment 81. The method of any one of embodiments 76-80, wherein the dose comprises between 1-20% (w/w) CBG.

Embodiment 82. The method of any one of embodiments 76-81, wherein the dose comprises between 5-15% (w/w) CBG.

Embodiment 83. The method of any one of embodiments 76-82, wherein the dose comprises between 10-12.5% (w/w) CBG.

Embodiment 84. The method of any one of embodiments 76-83, wherein the dose comprises about 11.7% (w/w) CBG.

Embodiment 85. The method of any one of embodiments 76-84, wherein the dose comprises between 1-20% (w/w) Δ8-THC.

Embodiment 86. The method of any one of embodiments 76-85, wherein the dose comprises between 5-15% (w/w) Δ8-THC.

Embodiment 87. The method of any one of embodiments 76-86, wherein the dose comprises between 7.5-15% (w/w) Δ8-THC.

Embodiment 88. The method of any one of embodiments 76-87, wherein the dose comprises about 8.1% (w/w) Δ8-THC.

Embodiment 89. The method of any one of embodiments 76-88, wherein the dose comprises between 0.01-2% (w/w) terpene component.

Embodiment 90. The method of any one of embodiments 76-89, wherein the dose comprises between 0.1-1% (w/w) terpene component.

Embodiment 91. The method of any one of embodiments 76-90, wherein the dose comprises about 0.4% (w/w) terpene component.

Embodiment 92. The method of any one of embodiments 76-91, wherein terpene component comprises at most one terpene.

Embodiment 93. The method of any one of embodiments 76-92, wherein terpene component comprises at least one terpene.

Embodiment 94. The method of any one of embodiments 76-93, wherein terpene component comprises at least two terpenes.

Embodiment 95. The method of any one of embodiments 76-94, wherein the terpene component comprises myrcene terpene.

Embodiment 96. The method of any one of embodiments 76-95, wherein the terpene component comprises linalool terpene.

Embodiment 97. The method of any one of embodiments 76-96, wherein the terpene component comprises myrcene terpene and linalool terpene.

Embodiment 98. The method of any one of embodiments 76-97, wherein the dose comprises between 0.01-1% (w/w) myrcene terpene.

Embodiment 99. The method of any one of embodiments 76-98, wherein the dose comprises between 0.1-0.5% (w/w) myrcene terpene.

Embodiment 100. The method of any one of embodiments 76-99, wherein the dose comprises about 0.2% (w/w) myrcene terpene.

Embodiment 101. The method of any one of embodiments 76-100, wherein the dose comprises between 0.01-1% (w/w) linalool terpene.

Embodiment 102. The method of any one of embodiments 76-101, wherein the dose comprises between 0.1-0.5% (w/w) linalool terpene.

Embodiment 103. The method of any one of embodiments 76-102, wherein the dose comprises about 0.2% (w/w) linalool terpene.

Embodiment 104. The method of any one of embodiments 76-103 wherein the dose comprises between 0.01-1% (w/w) linalool terpene and/or 0.01-1% (w/w) myrcene terpene.

Embodiment 105. The method of any one of embodiments 76-104, wherein the dose comprises between 0.1-0.5% (w/w) linalool terpene and/or 0.1-0.5% (w/w) myrcene terpene.

Embodiment 106. The method of any one of embodiments 76-105, wherein the dose comprises about 0.2% (w/w) linalool terpene and/or about 0.2% (w/w) myrcene terpene.

Embodiment 107. The method of any one of embodiments 76-106, wherein the dose comprises between 0.01-1% (w/w) sodium stearyl fumarate.

Embodiment 108. The method of any one of embodiments 76-107, wherein the dose comprises between 0.1-0.6% (w/w) sodium stearyl fumarate.

Embodiment 109. The method of any one of embodiments 76-108, wherein the dose comprises about 0.4% (w/w) sodium stearyl fumarate.

Embodiment 110. The method of any one of embodiments 76-109, wherein the dose comprises between 20-50% (w/w) colloidal silicon dioxide.

Embodiment 111. The method of any one of embodiments 76-110, wherein the dose comprises between 35-45% (w/w) colloidal silicon dioxide.

Embodiment 112. The method of any one of embodiments 76-111 wherein the dose comprises about 39% (w/w) colloidal silicon dioxide.

Embodiment 113. The method of any one of embodiments 76-112, wherein improved pain is measured by Daily Pain Numeric Rating Scale (NRS), Neuropathic Pain Symptom Inventory (NPSI), Short Form McGill Pain Questionnaire (SF-MPQ), Visual Analogue Scale (VAS), Daily Sleep Interference Scale Score (DSIS), Clinical Global Impression of Change (CGIC), Patient Global Impression of Change (PGIC), Baseline Hospital Anxiety and Depression Scale (HADS), weight gain, change in daily total activity, a change in the total daily amount of rescue medication, a change in the daily loss of balance, a change in the daily capture of sensory issues, and/or a change in the daily capture of muscular issues.

Embodiment 114. The method of any one of embodiments 76-113, wherein the pain improves by between 1-95%.

Embodiment 115. The method of any one of embodiments 76-114, wherein the pain improves by between 10-80%.

Embodiment 116. The method of any one of embodiments 76-115, wherein the pain improves by between 20-60%.

Embodiment 117. The method of any one of embodiments 76-116, wherein the pain improves by about 50%.

Embodiment 118. The method of any one of embodiments 76-117, wherein the subject has Type I diabetes.

Embodiment 119. The method of any one of embodiments 76-118, wherein the subject has Type II diabetes.

Embodiment 120. The method of any one of embodiments 76-119, wherein the subject takes at least two doses daily for at least 1 week.

Embodiment 121. The method of any one of embodiments 76-120, wherein the subject takes at least two doses daily for at least 2 weeks.

Embodiment 122. The method of any one of embodiments 76-121, wherein the subject takes at least two doses daily for at least 3 weeks.

Embodiment 123. The method of any one of embodiments 76-122, wherein the subject takes at least two doses daily for at least 4 weeks.

Embodiment 124. The method of any one of embodiments 76-123, wherein the subject takes at least two doses daily for at least 1 month.

Embodiment 125. The method of any one of embodiments 76-124, wherein the subject takes at least two doses daily for at least 2 months.

Embodiment 126. The method of any one of embodiments 76-125, wherein the subject takes at least two doses daily for at least 3 months.

Embodiment 127. The method of any one of embodiments 76-126, wherein the subject takes at least two doses daily for at least 4 months.

Embodiment 128. The method of any one of embodiments 76-127, wherein the subject takes at least two doses daily for at least 5 months.

Embodiment 129. The method of any one of embodiments 76-128, wherein the subject takes at least two doses daily for at least 6 months.

Embodiment 130. The method of any one of embodiments 76-129, wherein the subject takes at least two doses daily for at least 12 months.

Embodiment 131. The method of any one of embodiments 76-130, wherein the subject takes at least two doses daily for at least 24 months.

Embodiment 132. The method of any one of embodiments 76-131, wherein the subject takes at least two doses daily for at least 36 months.

Embodiment 133. The method of any one of embodiments 76-132, wherein at least three doses are taken daily.

Embodiment 134. The method of any one of embodiments 76-133, wherein at least four doses are taken daily.

Embodiment 134.1 A Cannabinoid Product comprising:
(a) a non-crystalline cannabinoid oil; and
(b) colloidal silicon dioxide particles.

Embodiment 135. The Cannabinoid Product of embodiment 134.1, wherein the non-crystalline cannabinoid oil and colloidal silicon dioxide particles are provided as a mixture.

Embodiment 136. A Cannabinoid Product comprising:
(a) a non-crystalline cannabinoid oil;
(b) colloidal silicon dioxide particles;
(c) a filler comprising silicified microcrystalline cellulose or mannitol;
(d) a disintegrant comprising sodium starch glycolate or croscarmellose sodium; and
(e) a lubricant comprising sodium stearyl fumarate.

Embodiment 137. The Cannabinoid Product of embodiment 136, wherein the non-crystalline cannabinoid oil and colloidal silicon dioxide particles are provided as a mixture.

Embodiment 138. The Cannabinoid Product of any of embodiments 136-137, comprising from about 10% to about 60% non-crystalline cannabinoid oil by weight.

Embodiment 139. The Cannabinoid Product of any of embodiments 136-138, comprising from about 19.4% to about 41.9% non-crystalline cannabinoid oil by weight.

Embodiment 140. The Cannabinoid Product of any of embodiments 136-139, wherein the filler is silicified microcrystalline cellulose.

Embodiment 141. The Cannabinoid Product of any of embodiments 136-140, comprising from about 30% to about 60% silicified microcrystalline cellulose by weight.

Embodiment 142. The Cannabinoid Product of any of embodiments 136-141, comprising about 46.6% silicified microcrystalline cellulose by weight.

Embodiment 143. The Cannabinoid Product of any of embodiments 136-142, wherein the filler is mannitol.

Embodiment 144. The Cannabinoid Product of any of embodiments 136-143, comprising from about 5% to about 60% mannitol by weight.

Embodiment 145. The Cannabinoid Product of any of embodiments 136-144, comprising from about 12.3% to about 43.4% mannitol by weight.

Embodiment 146. The Cannabinoid Product of any of embodiments 136-145, comprising from about 20% to about 70% colloidal silicon dioxide particles by weight.

Embodiment 147. The Cannabinoid Product of any of embodiments 136-146, comprising from about 29.1% to about 58.2% colloidal silicon dioxide particles by weight.

Embodiment 148. The Cannabinoid Product of any of embodiments 136-147, wherein the disintegrant is sodium starch glycolate.

Embodiment 149. The Cannabinoid Product of any of embodiments 136-148, comprising from about 0.5% to about 10% sodium starch glycolate by weight.

Embodiment 150. The Cannabinoid Product of any of embodiments 136-149, comprising from about 2.0% to about 5.5% sodium starch glycolate by weight.

Embodiment 151. The Cannabinoid Product of any of embodiments 136-150, wherein the disintegrant is croscarmellose sodium.

Embodiment 152. The Cannabinoid Product of any of embodiments 136-151, comprising from about 0.5% to about 5% croscarmellose sodium by weight.

Embodiment 153. The Cannabinoid Product of any of embodiments 136-152, comprising about 1.9% croscarmellose sodium by weight.

Embodiment 154. The Cannabinoid Product of any of embodiments 136-153, comprising from about 0.5% to about 10% sodium stearyl fumarate by weight.

Embodiment 155. The Cannabinoid Product of any of embodiments 136-154, comprising from about 1% to about 2.7% sodium stearyl fumarate by weight.

Embodiment 156. The Cannabinoid Product of any of embodiments 136-155, comprising:
(a) about 41.9% non-crystalline cannabinoid oil by weight;
(b) about 41.9% colloidal silicon dioxide particles by weight;
(c) about 12.3% mannitol by weight;
(d) about 2% sodium starch glycolate by weight; and
(e) about 2% sodium stearyl fumarate by weight.

Embodiment 157. The Cannabinoid Product of any of embodiments 136-156, comprising:
(a) about 22.1% non-crystalline cannabinoid oil by weight;
(b) about 58.2% colloidal silicon dioxide particles by weight;
(c) about 14.3% mannitol by weight;
(d) about 2.7% sodium starch glycolate by weight; and
(e) about 2.7% sodium stearyl fumarate by weight.

Embodiment 158. The Cannabinoid Product of any of embodiments 136-157, comprising:
(a) about 22.1% non-crystalline cannabinoid oil by weight;
(b) about 29.1% colloidal silicon dioxide particles by weight;
(c) about 40.7% mannitol by weight;
(d) about 5.5% sodium starch glycolate by weight; and
(e) about 2.7% sodium stearyl fumarate by weight.

Embodiment 159. The Cannabinoid Product of any of embodiments 136-158, comprising:
(a) about 22.1% non-crystalline cannabinoid oil by weight;
(b) about 29.1% colloidal silicon dioxide particles by weight;
(c) about 43.4% mannitol by weight;
(d) about 2.7% sodium starch glycolate by weight; and
(e) about 2.7% sodium stearyl fumarate by weight.

Embodiment 160. The Cannabinoid Product of any of embodiments 136-159, comprising:
(a) about 19.4% non-crystalline cannabinoid oil by weight;
(b) about 31.1% colloidal silicon dioxide particles by weight;
(c) about 46.6% silicified microcrystalline cellulose by weight;
(d) about 1.9% croscarmellose sodium by weight; and
(e) about 1% sodium stearyl fumarate by weight.

Embodiment 161. The Cannabinoid Product of any one of embodiments 136-160, further comprising a cannabinoid isolate.

Embodiment 162. The Cannabinoid Product of any one of embodiments 136-161, wherein the cannabinoid isolate is cannabidiol (CBD) isolate or cannabigerol (CBG) isolate.

Embodiment 163. The Cannabinoid Product of any one of embodiments 136-162, further comprising a flavoring.

Embodiment 164. The Cannabinoid Product of embodiments 136-163, wherein the flavoring is a terpene.

Embodiment 165. The Cannabinoid Product of embodiments 136-164, wherein the terpene is myrcene, linalool, or a combination thereof.

Embodiment 166. The Cannabinoid Product of any one of embodiments 136-165, wherein the non-crystalline cannabinoid oil is cannabinoid distillate.

Embodiment 167. The Cannabinoid Product of any one of embodiments 136-166, wherein the non-crystalline cannabinoid oil and the colloidal silicon dioxide particles are mixed in a high-shear mixer before the addition of the filler, disintegrant, or lubricant.

Embodiment 168. The Cannabinoid Product of any one of embodiments 136-167, wherein the lubricant is added after the filler and disintegrant.

Embodiment 169. The Cannabinoid Product of any one of embodiments 136-168, wherein the colloidal silicon dioxide particles have an average particle size of 20-60 μm.

Embodiment 170. The Cannabinoid Product of any one of embodiments 136-169, wherein the colloidal silicon dioxide particles contain pores with a volume of 1.5-1.9 milliliter (mL)/gram (g).

Embodiment 171. A Cannabinoid Product comprising:
(a) a non-crystalline cannabinoid oil;
(b) colloidal silicon dioxide particles; and
(c) a lubricant comprising sodium stearyl fumarate.

Embodiment 172. The Cannabinoid Product of embodiment 171, wherein the non-crystalline cannabinoid oil and colloidal silicon dioxide particles are provided as a mixture.

Embodiment 173. The Cannabinoid Product of any one of embodiments 171-172, comprising from about 0.5% to about 20% non-crystalline cannabinoid oil by weight.

Embodiment 174. The Cannabinoid Product of any one of embodiments 171-173, comprising from about 1% to about 10% non-crystalline cannabinoid oil by weight.

Embodiment 175. The Cannabinoid Product of any one of embodiments 171-174, comprising about 2% non-crystalline cannabinoid oil by weight.

Embodiment 176. The Cannabinoid Product of any one of embodiments 171-175, comprising about 8% non-crystalline cannabinoid oil by weight.

Embodiment 177. The Cannabinoid Product of any one of embodiments 171-176, comprising from about 25% to about 50%, from about 25% to about 35%, or from about 35% to about 45% colloidal silicon dioxide particles by weight.

Embodiment 178. The Cannabinoid Product of any one of embodiments 171-177, comprising about 32% colloidal silicon dioxide particles by weight.

Embodiment 179. The Cannabinoid Product of any one of embodiments 171-178, comprising about 39% colloidal silicon dioxide particles by weight.

Embodiment 180. The Cannabinoid Product of any one of embodiments 171-179, comprising from about 0.1% to about 1% sodium stearyl fumarate by weight.

Embodiment 181. The Cannabinoid Product of any one of embodiments 171-180, comprising about 0.4% sodium stearyl fumarate by weight.

Embodiment 182. The Cannabinoid Product of any one of embodiments 171-181, further comprising a cannabinoid isolate.

Embodiment 183. The Cannabinoid Product of any one of embodiments 171-182, wherein the cannabinoid isolate is cannabidiol (CBD) isolate or cannabigerol (CBG) isolate.

Embodiment 184. The Cannabinoid Product of any one of embodiments 171-183, comprising from 20% to about 60%, from about 20% to about 40%, or from about 40% to about 60% cannabinoid isolate by weight.

Embodiment 185. The Cannabinoid Product of any one of embodiments 171-184, further comprising a filler.

Embodiment 186. The Cannabinoid Product of any one of embodiments 171-185, wherein the filler is silicified microcrystalline cellulose.

Embodiment 187. The Cannabinoid Product of any one of embodiments 171-186, comprising from about 20% to about 45% filler by weight.

Embodiment 188. The Cannabinoid Product of any one of embodiments 171-187, comprising about 25% filler by weight.

Embodiment 189. The Cannabinoid Product of any one of embodiments 171-188, further comprising a flavoring.

Embodiment 190. The Cannabinoid Product of any one of embodiments 171-189, wherein the flavoring is a terpene.

Embodiment 191. The Cannabinoid Product of any one of embodiments 171-190, wherein the terpene is myrcene, linalool, or a combination thereof.

Embodiment 192. The Cannabinoid Product of any one of embodiments 171-191, comprising:
(a) about 8.1% non-crystalline cannabinoid oil by weight;
(b) about 39% colloidal silicon dioxide particles by weight; and
(c) about 0.4% sodium stearyl fumarate by weight.

Embodiment 193. The Cannabinoid Product of any one of embodiments 171-192, comprising:
(a) about 2.2% non-crystalline cannabinoid oil by weight of the Cannabinoid Product;
(b) about 32.4% colloidal silicon dioxide particles by weight; and
(c) about 0.4% sodium stearyl fumarate by weight.

Embodiment 194. The Cannabinoid Product of any one of embodiments 171-193, comprising:
(a) about 8.1% non-crystalline cannabinoid oil by weight;
(b) about 39% colloidal silicon dioxide particles by weight;
(c) about 0.4% sodium stearyl fumarate by weight;
(d) about 40.4% cannabidiol (CBD) isolate by weight;
(e) about 11.7% cannabigerol (CBG) isolate by weight;
(f) about 0.2% myrcene by weight; and
(g) about 0.2% linalool by weight.

Embodiment 195. The Cannabinoid Product of any one of embodiments 171-194, comprising:
(a) about 8.1% non-crystalline cannabinoid oil by weight;
(b) about 39% colloidal silicon dioxide particles by weight;
(c) about 0.4% sodium stearyl fumarate by weight;
(d) about 40.4% cannabidiol (CBD) isolate by weight; and
(e) about 11.7% cannabigerol (CBG) isolate by weight;

Embodiment 196. The Cannabinoid Product of any one of embodiments 171-195, comprising:
(a) about 2.2% non-crystalline cannabinoid oil by weight;
(b) about 32.4% colloidal silicon dioxide particles by weight;
(c) about 0.4% sodium stearyl fumarate by weight; and
(d) about 34.8% silicified microcrystalline cellulose by weight.

Embodiment 197. The Cannabinoid Product of any one of embodiments 171-196, wherein the non-crystalline cannabinoid oil is a cannabinoid distillate.

Embodiment 198. The Cannabinoid Product of any one of embodiments 171-197, wherein the non-crystalline cannabinoid oil and the colloidal silicon dioxide particles are mixed in a high-shear mixer before the addition of the lubricant.

Embodiment 199. The Cannabinoid Product of any one of embodiments 171-198, wherein the colloidal silicon dioxide particles have an average particle size of from about 20 μm to about 60 μm.

Embodiment 200. The Cannabinoid Product of any one of embodiments 171-199, wherein the colloidal silicon dioxide particles contain pores with a volume of from about 1.5 milliliter (mL)/gram (g) to about 1.9 mL/g.

Embodiment 201. A tablet comprising the Cannabinoid Product of any one of embodiments 171-200.

Embodiment 202. The tablet of embodiment 201, wherein the tablet has a hardness of from 50 Newtons (N) to 100 N.

Embodiment 203. The tablet of any one of embodiments 201-202, wherein the tablet has a hardness of about 65 N.

Embodiment 204. The tablet of any one of embodiments 201-203, wherein the tablet has a compression force of from 1 kiloNewtons (kN) to 30 kN.

Embodiment 205. The tablet of any one of embodiments 201-204, wherein the tablet has a compression force of from about 10 kN to 16 kN.

Embodiment 206. The tablet of any one of embodiments 201-205, wherein at least 18% of the tablet dissolves in fed state simulated intestinal fluid or fasted state simulated intestinal fluid within one hour.

Embodiment 207. The tablet of any one of embodiments 201-206, wherein from 60% to about 80% of the tablet dissolves.

Embodiment 208. The tablet of any one of embodiments 201-207, wherein from 70% to 80% of the tablet dissolves.

Embodiment 209. The tablet of any one of embodiments 201-208, wherein at least 36.5% of the tablet dissolves in polysorbate 80.

Embodiment 210. The tablet of any one of embodiments 201-209, wherein the tablet disintegrates in from 1 minute to about 10 minutes in 2% w/v sodium lauryl sulfate.

Embodiment 211. The tablet of any one of embodiments 201-210, wherein the tablet dissolves at least two times, at least three times, at least four times, at least five times, or at least ten times faster than a tablet containing a Cannabinoid Product lacking colloidal silicon dioxide particles.

Embodiment 212. The tablet of any one of embodiments 201-211, wherein the tablet has a weight of from about 100 to about 800 mg.

Embodiment 213. The tablet of any one of embodiments 201-212, wherein the tablet comprises from about 0.1% to about 30% of non-crystalline cannabinoid oil by weight.

Embodiment 214. The tablet of any one of embodiments 201-213, wherein the tablet is an orodispersable tablet.

Embodiment 215. A capsule comprising the Cannabinoid Product of any one of embodiments 201-214.

Embodiment 216. The capsule of embodiment 215, wherein the capsule comprises a shell comprising gelatin or hydroxypropylmethylcellulose.

Embodiment 217. The capsule of any one of embodiments 215-216, wherein the capsule disintegrates within about 2 minutes in an aqueous solvent.

Embodiment 218. The capsule of any one of embodiments 215-217, wherein at least 18% of the capsule dissolves in fed state simulated intestinal fluid or fasted state simulated intestinal fluid within one hour.

Embodiment 219. The capsule of any one of embodiments 215-218, wherein from 18% to about 60% of the capsule dissolves.

Embodiment 220. The capsule of any one of embodiments 215-219, wherein from 18.1% to 42.3% of the capsule dissolves.

Embodiment 221. The capsule of any one of embodiments 215-220, wherein at least 36.5% of the capsule dissolves within one hour.

Embodiment 222. The capsule of any one of embodiments 215-221, wherein the capsule dissolves at least two times, at least three times, at least four times, at least five times, or at least ten times faster than a capsule containing a Cannabinoid Product lacking colloidal silicon dioxide particles.

Embodiment 223. The capsule of any one of embodiments 215-222, wherein the capsule comprises from about 1% to about 50% non-crystalline cannabinoid oil by weight.

Embodiment 224. A method of making the Cannabinoid Product of any one of embodiments 215-223, comprising:
(a) mixing the non-crystalline cannabinoid oil and colloidal silicon dioxide particles;
(b) adding the filler and disintegrant to the mixture of non-crystalline cannabinoid oil and colloidal silicon dioxide particles; and
(c) adding the lubricant.

Embodiment 225. The method of embodiment 224, wherein step (a) is performed before step (b), and step (b) is performed before step (c).

Embodiment 226. The method of any one of embodiments 224-225, wherein the non-crystalline cannabinoid oil is heated before the extract oil is mixed with the colloidal silicon dioxide particles.

Embodiment 227. The method of any one of embodiments 224-226, wherein the non-crystalline cannabinoid oil and colloidal silicon dioxide particles are heated after mixing.

Embodiment 228. The method of any one of embodiments 224-227, wherein the non-crystalline cannabinoid oil is diluted in a solvent before mixing with colloidal silicon dioxide particles.

Embodiment 229. The method of any one of embodiments 224-228, wherein the solvent is ethanol.

Embodiment 230. The method of any one of embodiments 224-229, wherein the non-crystalline cannabinoid oil is heated to at least 30° C., at least 40° C., at least 50° C., at least 60° C., at least 70° C., at least 80° C., or at least 90° C.

Embodiment 231. The method of any one of embodiments 224-230, wherein the non-crystalline cannabinoid oil is added to the colloidal silicon dioxide particles.

Embodiment 232. The method of any one of embodiments 224-231, wherein the colloidal silicon dioxide particles are added to the cannabinoid non-crystalline cannabinoid oil.

Embodiment 233. The method of any one of embodiments 224-232, wherein the non-crystalline cannabinoid oil and colloidal silicon dioxide particles are mixed in a high-shear mixer.

Embodiment 234. The method of any one of embodiments 224-233, wherein the non-crystalline cannabinoid oil and colloidal silicon dioxide particles are mixed for at least 30 seconds, at least 1 minute, at least 5 minutes, at least 10 minutes, at least 15 minutes, or at least 20 minutes.

Embodiment 235. The method of any one of embodiments 224-234, wherein the non-crystalline cannabinoid oil and colloidal silicon dioxide particles are mixed at from 1000 to 2000 revolutions per minute (rpm).

Embodiment 236. The method of any one of embodiments 224-235, wherein the high-shear mixer is a grinder.

Embodiment 237. A method of making a tablet comprising compressing a Cannabinoid Product of any one of embodiments 171-200 in a tablet press.

Embodiment 238. The method of embodiment 237, wherein the tablet is about 100 to 800 mg.

Embodiment 239. The method of any one of embodiments 237-238, wherein the tablet comprises from about 0.1% to about 30% of non-crystalline cannabinoid oil by weight.

Embodiment 240. The method of any one of embodiments 237-239, wherein the tablet is an orodispersable tablet.

Embodiment 241. A method of making the Cannabinoid Product of any one of embodiments 171-200, comprising:
(a) mixing the non-crystalline cannabinoid oil and colloidal silicon dioxide particles; and
(b) adding the lubricant.

Embodiment 242. The method of embodiment 241, further comprising:
(c) adding a filler.

Embodiment 243. The method of any one of embodiments 241-242, wherein the filler is silicified microcrystalline cellulose.

Embodiment 244. The method of any one of embodiments 241-243, wherein the non-crystalline cannabinoid oil is diluted in a solvent before mixing with colloidal silicon dioxide particles.

Embodiment 245. The method of any one of embodiments 241-244, wherein the solvent is ethanol.

Embodiment 246. The method of any one of embodiments 241-245, wherein step (a) is performed before step (b).

Embodiment 247. The method of any one of embodiments 241-246, wherein step (a) is performed before step (b), and step (b) is performed before step (c).

Embodiment 248. The method of any one of embodiments 241-247, wherein step (a) is performed before step (c), and step (c) is performed before step (b).

Embodiment 249. The method of any one of embodiments 241-248, wherein the non-crystalline cannabinoid oil is heated before the oil is mixed with the colloidal silicon dioxide particles.

Embodiment 250. The method of any one of embodiments 241-249, wherein the non-crystalline cannabinoid oil and colloidal silicon dioxide particles are heated after mixing.

Embodiment 251. The method of any one of embodiments 241-250, wherein the non-crystalline cannabinoid oil is heated to at least 30° C., at least 40° C., at least 50° C., at least 60° C., at least 70° C., at least 80° C., or at least 90° C.

Embodiment 252. The method of any one of embodiments 241-251, wherein the non-crystalline cannabinoid oil is added to the colloidal silicon dioxide particles.

Embodiment 253. The method of any one of embodiments 241-252, wherein the colloidal silicon dioxide particles are added to the cannabinoid non-crystalline cannabinoid oil.

Embodiment 254. The method of any one of embodiments 241-253, wherein the non-crystalline cannabinoid oil and colloidal silicon dioxide particles are mixed in a high-shear mixer.

Embodiment 255. The method of any one of embodiments 241-254, wherein the non-crystalline cannabinoid oil and colloidal silicon dioxide particles are mixed for at least 30 seconds, at least 1 minute, at least 5 minutes, at least 10 minutes, at least 15 minutes, or at least 20 minutes.

Embodiment 256. The method of any one of embodiments 241-255, wherein the non-crystalline cannabinoid oil and colloidal silicon dioxide particles are mixed at a speed of from about 1000 revolutions per minute (rpm) to about 2000 rpm.

Embodiment 257. The method of any one of embodiments 241-256, wherein the lubricant is mixed with a mixture containing non-crystalline cannabinoid oil, colloidal silicon dioxide particles, filler, and disintegrant for from 1 minute (min) to about 2 min.

Embodiment 258. The method of any one of embodiments 241-257, wherein the high-shear mixer is a grinder.

Embodiment 259. A method of making a capsule, comprising filling a capsule shell with the Cannabinoid Product of any one of embodiments 171-200.

Embodiment 260. The method of embodiment 259, wherein the capsule shell comprises gelatin or hydroxypropylmethylcellulose.

Embodiment 261. The method of any one of embodiments 259-260, wherein the capsule comprises from about 1% to about 50% non-crystalline cannabinoid oil by weight.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. The following documents are incorporated by reference herein in their entirety: International Application No. PCT/US23/68501, International Publication No. 2003/082246; U.S. Publication No. 2019/0060381; U.S. Publication No. 2017/0368020; International Publication No. 2019/216832; U.S. Pat. No. 11,077,086; International Publication No. 2021/151169; Sperry et al. Precis. Nanomed. 2021 December; 4(4):851-878; Pandey et al. Pharm Dev Technol. 2013 February; 18(1):296-304; and Sopper et al. Sci Pharm. 2021, 89, 35.

What is claimed is:

1. A pharmaceutical composition for treating pain in a subject with diabetic neuropathy, the pharmaceutical composition comprising:
(a) a cannabinoid;
(b) colloidal silicon dioxide; and,
(c) terpene component.

2. A method of treating pain in a subject with diabetic neuropathy by administering to the subject a dose at least twice daily, the dose comprising:
(a) a cannabinoid;
(b) colloidal silicon dioxide; and,
(c) terpene component,
wherein, the subject's pain improves compared to a subject that is not administered the dose.

3. The method of claim 2, wherein the cannabinoid comprises Cannabidiol (CBD), Cannabigerol (CBG), and Δ8-Tetrahydrocannabinol (Δ8-THC).

4. The method of claim 2, wherein the dose comprises from 20-50% (w/w) CBD.

5. The method of claim 2, wherein the dose comprises a lubricant.

6. The method of claim 3, wherein the dose comprises from 1-20% (w/w) CBG.

7. The method of claim 2, wherein the dose comprises Sodium Stearyl Fumarate.

8. The method of claim 3, wherein the dose comprises from 1-20% (w/w) Δ8-THC.

9. The method of claim 2, wherein the dose comprises from 0.01-2% (w/w) terpene component.

10. The method of claim 2, wherein the terpene component comprises myrcene terpene and linalool terpene.

11. The method of claim 10, wherein the dose comprises from 0.01-1% (w/w) myrcene terpene.

12. The method of claim 10, wherein the dose comprises from 0.01-1% (w/w) linalool terpene.

13. The method of claim 10, wherein the dose comprises from 0.1-0.5% (w/w) linalool terpene and/or 0.1-0.5% (w/w) myrcene terpene.

14. The method of claim 2, wherein the dose comprises from 0.01-1% (w/w) sodium stearyl fumarate.

15. The method of claim 2, wherein the dose comprises from 20-50% (w/w) colloidal silicon dioxide.

16. The method of claim 2, wherein the dose comprises from 35-45% (w/w) colloidal silicon dioxide.

17. The method of claim 2, wherein improved pain is measured by Daily Pain Numeric Rating Scale (NRS), Neuropathic Pain Symptom Inventory (NPSI), Short Form McGill Pain Questionnaire (SF-MPQ), Visual Analogue Scale (VAS), Daily Sleep Interference Scale Score (DSIS), Clinical Global Impression of Change (CGIC), Patient Global Impression of Change (PGIC), Baseline Hospital Anxiety and Depression Scale (HADS), weight gain, change in daily total activity, a change in the total daily amount of rescue medication, a change in the daily loss of balance, a change in the daily capture of sensory issues, and/or a change in the daily capture of muscular issues.

18. The method of claim 17, wherein the pain improves by from 1-95%.

19. The method of claim 17, wherein the pain improves by from 10-80%.

20. The method of claim 2, wherein the subject takes at least two doses daily for at least 1 week.
